(19) United States
(12) Patent Application Publication
(10) Pub. No.: US 2025/0034656 A1
(43) Pub. Date: Jan. 30, 2025

(54) METHODS FOR MONITORING IMPACT OF MICROBIOTA TRANSFER THERAPY IN AUTISM SPECTRUM DISORDER (ASD)

(71) Applicant: Arizona Board of Regents on Behalf of Arizona State University, Tempe, AZ (US)

(72) Inventors: Rosa Krajmalnik-Brown, Chandler, AZ (US); James Adams, Tempe, AZ (US); Khemlal Nirmalkar, Tempe, AZ (US)

(73) Assignee: Arizona Board of Regents on Behalf of Arizona State University, Tempe, AZ (US)

(21) Appl. No.: 18/714,217

(22) PCT Filed: Nov. 30, 2022
(86) PCT No.: PCT/US2022/080704

§ 371 Date: May 29, 2024

Related U.S. Application Data
(60) Provisional application No. 63/284,315, filed on Nov. 30, 2021.

(51) Int. Cl. C12Q 1/689

(52) U.S. Cl. CPC C12Q 1/689; C12Q 2600/106; C12Q 2600/156

(57) ABSTRACT

The present disclosure relates to methods for monitoring effectiveness of a treatment of an autism spectrum disorder (ASD) by analyzing an ASD patient's gut microbiota based on stool samples. Also provided are software for implementing the methods, and kits for performing the methods.

### CROSS-REFERENCE TO RELATED APPLICATIONS

This application claims priority from Provisional Application No. 63/284,315 filed Nov. 30, 2021, the entire contents of which are hereby incorporated by reference.

### BACKGROUND

The present disclosure relates to methods of treating autism spectrum disorder (ASD). Autism spectrum disorder (ASD) is a complex neurodevelopmental condition characterized by widespread abnormalities of social interactions and communication, as well as restricted interests and repetitive behaviors. ASD typically appears during the first three years of life and manifests in characteristic symptoms or behavioral traits. A diagnosis of ASD now includes several conditions that used to be diagnosed separately: autistic disorder, pervasive developmental disorder not otherwise specified (PDD-NOS), and Asperger syndrome. All of these conditions are now encompassed by the diagnostic criteria for autism spectrum disorder as set forth in the American Psychiatric Association's Diagnostic & Statistical Manual of Mental Disorders, Fifth Edition (DSM-V).

In addition to the spectrum of symptoms seen within these principal diagnostic criteria, ASD individuals display a wide range of neurological comorbidities, including intellectual disability, epilepsy, and anxiety and mood disorders, as well as non-neurological comorbidities, including blood hyperserotonemia, immune dysregulation, and GI dysfunction (e.g., chronic constipation, diarrhea, abdominal pain, and gastroesophageal reflux).

To date, there are no FDA-approved treatments for reducing or eliminating the core symptoms of autism spectrum disorder. The only two medications approved by the FDA for treating autism, risperidone (sold under Risperdal®) and aripiprazole (sold under Abilify®), are specifically indicated for reducing irritability in subjects having ASD. Further lacking are methods for monitoring candidate treatments for ASD. Accordingly, there remains a need for improved methods for treating and reducing the severity and incidence of symptoms associated with autism spectrum disorder, and methods for monitoring the functional impact of treatments.

### SUMMARY

In some aspects, the current disclosure encompasses a method for monitoring effectiveness of a treatment of an autism spectrum disorder (ASD) in a subject in need thereof, comprising the steps of (a) obtaining or having obtained a stool sample from the subject after a treatment period; (b) sequencing the stool sample or portion thereof, wherein the sequencing comprises a shotgun metagenomic sequencing; (c) determining a relative abundance level of at least one microorganism in the subject's gut microbiome using data from the shotgun metagenomic sequencing; (d) comparing the relative abundance level of the at least one microorganism in the subject's gut microbiome with an abundance baseline to determine a difference between the abundance baseline and the relative abundance level after the treatment period; wherein the difference between the relative abundance and the abundance baseline after the treatment period is indicative of the effectiveness of the treatment. In some aspects the treatment is a Microbiota Transfer Therapy (MTT). In some aspects, the baseline abundance baseline is a relative abundance level of the microorganism in a stool sample from the subject prior to the treatment. In some aspects, the abundance baseline is a relative abundance level of the microorganism in a stool sample from a subject not diagnosed with ASD. In some aspects, the steps (c)-(d) are implemented using a computer software. In some aspects, the microorganism is selected from a group consisting of Prevotella dentalis, Prevotella enoeca, Prevotella oris, Prevotella meloninogenica, Prevotella denticola, Prevotella fusca, Prevotella intermedia, Prevotella ruminicola, Bifidobacterium bifidum, Bifidobacterium angulatum, Selenomonas sp., Selenomonas sp. oral taxon 136, Alistipes finegoldii, Lactobacillus vaginalis and Desulfovibrio sp. and Desulfovibrio piger or any combination thereof. In some aspects, the relative abundance level of the microorganism is higher than the baseline relative abundance level. In some aspects, the stool sample from the subject is obtained after 5 weeks-5 years, or about 10 weeks-2 years of the treatment. In some aspects, the stool sample from the subject is obtained during the treatment.

In some aspects, the current disclosure also encompasses a method for monitoring the effectiveness of a treatment of an autism spectrum disorder (ASD) in a subject in need thereof, comprising the steps of (a) obtaining or having obtained a stool sample from the subject after a period of administration of the treatment; (b) sequencing the stool sample or portion thereof, wherein the sequencing comprises a shotgun metagenomic sequencing; (c) determining a relative abundance level of at least one metabolic gene in the sample using the shotgun metagenomic sequencing data; (d) comparing the relative abundance level of the at least one gene with an abundance baseline of the gene(s) to determine a difference between the abundance baseline and the gene relative abundance level after the treatment period; wherein the difference between the gene relative abundance level and the abundance baseline after the treatment period is indicative of the effectiveness of the treatment. In some aspects, the steps (c)-(d) are implemented using a computer software. In some aspects the treatment is a Microbiota Transfer Therapy (MTT). In some aspects, the abundance baseline is a relative abundance level of the gene in a stool sample from the subject prior to the treatment. In some aspects, the abundance baseline relative abundance level is an relative abundance level of the gene in a stool sample from a subject not diagnosed with ASD. In some aspects, the relative abundance level of the at least one gene is higher than the baseline relative abundance level, non-limiting examples of such genes include a folate biosynthesis (K04094), vitamin B-12 synthesis (K02499), oleic acid synthesis (K10254), sulfur metabolism (dissimilatory sulfate reduction) aprB (K00395) or an oxidative stress protection gene (K05919, K07304) or any combination thereof. In some aspects of the method, the relative abundance level of the at least one gene is lower than the baseline relative abundance level, non-limiting examples of which include urease accessary protein ureH, ureD (K03190), urea transporter utp (K08717), glutamate metabolism FTCD (K13990), or ornithine and arginine biosynthesis argE (K01438) or any combination thereof.

In some aspects, the stool sample from the subject is obtained after 5 weeks-5 years of the treatment, or about 10 weeks-2 years. In some aspects, the stool sample is obtained from the subject during the treatment.

In some aspects, the current disclosure also encompasses a kit for monitoring effectiveness of a treatment of an autism spectrum disorder (ASD) in a subject in need thereof, comprising (a) container for obtaining a stool sample from the subject, (b) reagents and containers for conducting a shotgun metagenomic sequencing, and (c) instructions for use.

In some aspects, the current disclosure also encompasses a method for treating an autism spectrum disorder (ASD) in a subject in need thereof, comprising: (a) administering or having administered to the subject a pharmaceutical composition comprising a fecal microbiota preparation; (b) obtaining or having obtained a stool sample from the subject after a treatment period; (c) sequencing the stool sample or portion thereof, wherein the sequencing comprises a shotgun metagenomic sequencing; (d) determining a relative abundance level of at least one microorganism in the subject's gut microbiome using data from the shotgun metagenomic sequencing; (e) comparing the relative abundance level of the at least one microorganism in the subject's gut microbiome with an abundance baseline to determine a difference between the abundance baseline and the relative abundance level after the treatment period; (f) making a determination of the effectiveness of the treatment based on the difference between the relative abundance and the abundance baseline after the treatment period. In some aspects, the method for treating an autism spectrum disorder (ASD) in a subject in need thereof comprises modifying the gut microbiome in the subject by administering a fecal microbiota preparation, wherein the modifying is determined by determining a microorganism and/or a gene abundance in the subject's gut and comparing to a microorganism and/or gene abundance of a neurotypical control subject, wherein the microorganism abundance is a relative abundance level of at least one microorganism in the subject's gut microbiome. In some aspects of the method disclosed herein, the at least one microorganism is selected from Prevotella dentalis, Prevotella enoeca, Prevotella oris, Prevotella meloninogenica, Prevotella denticola, Prevotella fusca, Prevotella intermedia, Prevotella ruminicola, Bifidobacterium bifidum, Bifidobacterium angulatum, Selenomonas sp. Selenomonas sp. oral taxon 136, Lactobacillus vaginalis, Alistipes finegoldii, Desulfovibrio sp. and Desulfovibrio piger or any combination thereof.

In some aspects, the method further comprises (g) determining a relative gene abundance level of at least one metabolic gene in the sample using the shotgun metagenomic sequencing data; (h) comparing the relative gene abundance level of the at least one gene with an abundance baseline of the gene to determine a difference between the abundance baseline and the relative gene abundance level after the treatment period. In some aspects, the at least one gene is a folate biosynthesis (K04094), vitamin B-12 synthesis (K02499), oleic acid synthesis (K10254), sulfur metabolism (dissimilatory sulfate reduction) aprB (K00395), an oxidative stress protection gene (K05919, K07304), ureH, ureD (K03190), urea transporter utp (K08717), glutamate metabolism FTCD (K13990), or ornithine and arginine biosynthesis argE (K01438) or any combination thereof.

### BRIEF DESCRIPTION OF THE DRAWINGS

Aspects of the present inventive concept are illustrated by way of example in which like reference numerals indicate similar elements and in which:

FIG. 1 is an overview of Microbiota Transfer Therapy (MTT) trial in ASD and study time points. hd—high dose; ld—low dose; SHGM—standardized human gut microbiota; WGS—whole-genome sequencing; MB—metabolomics; CA—clinical assessment (includes all ASD- and GI-associated symptoms).

FIG. 2A is a graph showing the Shannon diversity index before and after MTT in children with ASD. Each dot represents a study participant. Dark dashed lines represent the mean of maintenance (n=2) and light, the median of major donors (n=5). TD: Typically Developing. * Asterisk represent significant differences between ASD Baseline, and the other groups (*p<0.05, ns not significant, all p-values are BH method FDR corrected).

FIG. 2B is a graph showing the KEGG Orthologs between groups before and after MTT in children with ASD. Each dot represents a study participant. Dark dashed lines represent the mean of maintenance (n=2) and light, the median of major donors (n=5). TD: Typically Developing. * Asterisk represent significant differences between ASD Baseline, and the other groups (*p<0.05, ns not significant, all p-values are BH method FDR corrected).

FIG. 3A provides the Jaccard distance dissimilarity index before and after MTT in children with ASD: (left) bacterial community and (right) KEGG Orthologs between groups. Each dot represents a study participant. TD: Typically Developing, Main Donor: Maintenance donor, ANOSIM: Analysis of similarities.

FIG. 3B provides the beta-diversity before and after MTT in children with ASD. The Bray-Curtis distance dissimilarity index of (left) bacteria and (right) KEGG Orthologs between groups. Each dot represents an ASD individual. ASD: Autism Spectrum Disorders, TD: Typically Developing, MaintDonor: Maintenance donor. ANOSIM (Analysis of similarities) was used for the statistical comparison between ASD Baseline and other groups.

FIG. 4 provides the univariate comparison of the (z-score) relative abundance of gut bacteria for the ASD Baseline group vs. all other groups. Top 30 bacterial species in three clusters that had significantly different relative abundance at Baseline vs. TD (cutoff p<0.01, adjusted p<0.05). Median of bacterial relative abundance was used to construct the heatmap for each group. * Single asterisk indicates p<0.05, ** double asterisks indicate p<0.01, triple *** asterisks indicate p<0.001. All p-values are FDR-corrected. ASD: autism spectrum disorder; TD: typically developing.

FIG. 5A shows a graph of the univariate comparison of the relative abundance (after log₁₀ transformation) of P. denatalis of ASD Baseline vs. MTT (10 wk, 2 yr) and TD. Upper-dashed lines represent the mean of maintenance (n=2) and lower represent median of major donors (n=5). Each dark dot represents one ASD individual and light grey-colored dots represent TD. Asterisks represent significant differences between ASD Baseline and the other groups (** double asterisks indicate p<0.01; ns: not significant; all p-values are FDR-corrected). ASD: autism spectrum disorder, TD: typically developing.

FIG. 5B shows a graph of the univariate comparison of the relative abundance (after log₁₀ transformation) of P. enoeca of ASD Baseline vs. MTT (10 wk, 2 yr) and TD. Upper-dashed lines represent the mean of maintenance (n=2) and lower represent median of major donors (n=5). Each dark dot represents one ASD individual and light grey-colored dots represent TD. Asterisks represent significant differences between ASD Baseline and the other groups (** double asterisks indicate p<0.01; ns: not significant; all p-values are FDR-corrected). ASD: autism spectrum disorder, TD: typically developing.

FIG. 5C shows a graph of the univariate comparison of the relative abundance (after log₁₀ transformation) of P. oris of ASD Baseline vs. MTT (10 wk, 2 yr) and TD. Upper-dashed lines represent the mean of maintenance (n=2) and lower represent median of major donors (n=5). Each dark dot represents one ASD individual and light grey-colored dots represent TD. Asterisks represent significant differences between ASD Baseline and the other groups (** double asterisks indicate p<0.01; ns: not significant; all p-values are FDR-corrected). ASD: autism spectrum disorder, TD: typically developing.

FIG. 5D shows a graph of the univariate comparison of the relative abundance (after log₁₀ transformation) of P. meloninogenica of ASD Baseline vs. MTT (10 wk, 2 yr) and TD. Upper-dashed lines represent the mean of maintenance (n=2) and lower represent median of major donors (n=5). Each dark dot represents one ASD individual and light grey-colored dots represent TD. Asterisks represent significant differences between ASD Baseline and the other groups (** double asterisks indicate p<0.01; ns: not significant; all p-values are FDR-corrected). ASD: autism spectrum disorder, TD: typically developing.

FIG. 6A shows a graph of the univariate comparison of the relative abundance (after log₁₀ transformation) of Bifidobacterium bifidum for ASD Baseline vs. all other groups. Upper-dashed lines represent the mean of maintenance donors (n=2) and lower represents the median of major donors (n=5). Each dark dot represents one ASD individual and light grey-dots represent TD. Asterisks represent significant differences between ASD Baseline and the other groups (* single asterisk indicates p<0.05; ** double asterisks indicate p<0.01; triple *** asterisks indicate p<0.001; #: statistically significant only after log 10 transformation; ns: not significant; all p-values are FDR-corrected). ASD: autism spectrum disorder; TD: typically developing.

FIG. 6B shows a graph of the univariate comparison of the relative abundance (after log₁₀ transformation) of Desulfovibrio piger for ASD Baseline vs. all other groups. Upper-dashed lines represent the mean of maintenance donors (n=2) and lower represents the median of major donors (n=5). Each dark dot represents one ASD individual and light grey-colored dots represent TD. Asterisks represent significant differences between ASD Baseline and the other groups (* single asterisk indicates p<0.05; ** double asterisks indicate p<0.01; triple *** asterisks indicate p<0.001; #: statistically significant only after log 10 transformation; ns: not significant; all p-values are FDR-corrected). ASD: autism spectrum disorder; TD: typically developing.

FIG. 6C shows a graph of the univariate comparison of the relative abundance (after log₁₀ transformation) of Lactobacillus vaginalis for ASD Baseline vs. all other groups. Upper-dashed lines represent the mean of maintenance donors (n=2) and lower represents the median of major donors (n=5). Each dark dot represents one ASD individual and light grey-colored dots represent TD. Asterisks represent significant differences between ASD Baseline and the other groups (* single asterisk indicates p<0.05; ** double asterisks indicate p<0.01; triple *** asterisks indicate p<0.001; #: statistically significant only after log 10 transformation; ns: not significant; all p-values are FDR-corrected). ASD: autism spectrum disorder; TD: typically developing.

FIG. 6D shows a graph of the univariate comparison of the relative abundance (after log₁₀ transformation) of) Alistipes finegoldii for ASD Baseline vs. all other groups. Here, for A. finegoldii, the lines representing the mean of maintenance donors (n=2) and median of major donors (n=5) overlap. Each dark dot represents one ASD individual and light grey-colored dots represent TD. Asterisks represent significant differences between ASD Baseline and the other groups (* single asterisk indicates p<0.05; ** double asterisks indicate p<0.01; triple *** asterisks indicate p<0.001; #: statistically significant only after log₁₀ transformation; ns: not significant; all p-values are FDR-corrected). ASD: autism spectrum disorder; TD: typically developing.

FIG. 7A is a graph showing the univariate comparison of the relative abundance (after log 10 transformation) of P. denticola of ASD Baseline vs. MTT (10 wk, 2 yr) and TD. Upper dashed lines represent the mean of maintenance (n=2) and lower for median of major donors (n=5). Each dark dot represents one ASD individual and light grey colored dots represent TD. Asterisks represent significant differences between ASD Baseline, and the other groups (*Single asterisk indicates p<0.05, **double asterisks indicate p<0.01, ns not significant, all p-values are FDR corrected).

FIG. 7B is a graph showing the univariate comparison of the relative abundance (after log 10 transformation) of P. fusca of ASD Baseline vs. MTT (10 wk, 2 yr) and TD. Upper dashed lines represent the mean of maintenance (n=2) and lower for median of major donors (n=5). Each dark colored dot represents one ASD individual and light grey colored dots represent TD. Asterisks represent significant differences between ASD Baseline, and the other groups (*Single asterisk indicates p<0.05, **double asterisks indicate p<0.01, ns not significant, all p-values are FDR corrected).

FIG. 7C is a graph showing the univariate comparison of the relative abundance (after log 10 transformation) of P. intermedia of ASD Baseline vs. MTT (10 wk, 2 yr) and TD.

Upper dashed lines represent the mean of maintenance (n=2) and lower for median of major donors (n=5). Each dark dot represents one ASD individual and light grey colored dots represent TD. Asterisks represent significant differences between ASD Baseline, and the other groups (*Single asterisk indicates p<0.05, **double asterisks indicate p<0.01, ns not significant, all p-values are FDR corrected).

FIG. 7D is a graph showing the univariate comparison of the relative abundance (after log 10 transformation) of P. ruminicola of ASD Baseline vs. MTT (10 wk, 2 yr) and TD. Upper dashed lines represent the mean of maintenance (n=2) and lower for median of major donors (n=5). Each dark dot represents one ASD individual and light grey colored dots represent TD. Asterisks represent significant differences between ASD Baseline, and the other groups (*Single asterisk indicates p<0.05, **double asterisks indicate p<0.01, ns not significant, all p-values are FDR corrected).

FIG. 7E is a graph showing the univariate comparison of the relative abundance (after log 10 transformation) of Bifidobacterium angulatum of ASD Baseline vs. MTT (10 wk, 2 yr) and TD. Upper dashed lines represent the mean of maintenance (n=2) and lower for median of major donors (n=5). Each dark dot represents one ASD individual and light grey colored dots represent TD. Asterisks represent significant differences between ASD Baseline, and the other groups (*Single asterisk indicates p<0.05, **double asterisks indicate p<0.01, ns not significant, all p-values are FDR corrected).

FIG. 8 is a heatmap of KOs that were significantly lower (KO Cluster-I) or higher (KO Cluster-II) in ASD baseline vs. TD. The heatmap also shows how KOs changed after MTT compared to Baseline, and generally became more similar to the TD group. The median of bacterial relative abundance was used to construct the heatmap for each group. * Single asterisk indicates p<0.05, ** double asterisks indicate p<0.01, triple *** asterisks indicate p<0.001. Univariate statistical comparisons were made for ASD Baseline vs. all other groups. All p-values are FDR-corrected. ASD: autism spectrum di-order; TD: typically developing.

FIG. 9A is a graph showing the univariate comparison of the relative abundance (after log₁₀ transformation) of gut microbiome genes/KO K04094 folate-dependent ribothymidyl synthase that changed significantly after MTT in ASD and became more similar to gene profiles in TD. Upper-dashed lines represent the mean of maintenance (n=2) and lower represent the median of major donors (n=5). Dark Colored dots represent ASD individuals and light grey-colored dots represent TD. Asterisks represent significant differences between ASD Baseline and the other groups (* single asterisk indicates p<0.05; ** double asterisks indicate p<0.01; triple *** asterisks indicate p<0.001; ns: not significant; all p-values are FDR-corrected). ASD: autism spectrum disorder; TD: typically developing.

FIG. 9B is a graph showing the univariate comparison of the relative abundance (after log₁₀ transformation) of gut microbiome genes/KO K10254: oleate hydratase that changed significantly after MTT in ASD and became more similar to gene profiles in TD. Upper-dashed lines represent the mean of maintenance (n=2) and lower represent the median of major donors (n=5). Dark Colored dots represent ASD individuals and light grey-colored dots represent TD. Asterisks represent significant differences between ASD Baseline and the other groups (* single asterisk indicates p<0.05; ** double asterisks indicate p<0.01; triple *** asterisks indicate p<0.001; ns: not significant; all p-values are FDR-corrected). ASD: autism spectrum disorder; TD: typically developing.

FIG. 9C is a graph showing the univariate comparison of the relative abundance (after log₁₀ transformation) of gut microbiome genes/KO K02499: tetrapyrrole methylase that changed significantly after MTT in ASD and became more similar to gene profiles in TD. Upper-dashed lines represent the mean of maintenance (n=2) and lower represent the median of major donors (n=5). Dark Colored dots represent ASD individuals and light grey-colored dots represent TD. Asterisks represent significant differences between ASD Baseline and the other groups (* single asterisk indicates p<0.05; ** double asterisks indicate p<0.01; triple *** asterisks indicate p<0.001; ns: not significant; all p-values are FDR-corrected). ASD: autism spectrum disorder; TD: typically developing.

FIG. 10 shows the univariate comparison of the relative abundance (after log₁₀ transformation) of gut microbiome genes/KOs that encode enzymes involved in oxygen detoxification and oxidative stress before and after MTT in ASD in comparison with TD. (top left) oxidative stress protection and detoxification of reactive oxygen species; K05919 (dfx gene, SOR): superoxide reductase. (bottom left) K07304 (msrA): pep-tide-methionine (S)-S-oxide reductase. (right) Illustration of enzymatic reactions of SOR and msrA KOs. Dashed lines represent the median of donors. Upper-dashed lines represent the mean of maintenance (n=2) and lower represent the median of major donors (n=5). Dark colored dots represent ASD individuals and light grey-colored dots represent TD. Asterisks represent significant differences between ASD Baseline and the other groups (* single asterisk indicates p<0.05; ** double asterisks indicate p<0.01; ns: not significant; all p-values are FDR-corrected). ASD: autism spectrum disorder; TD: typically developing.

FIG. 11 is an overview of sulfur metabolism. Shaded KO K01082 converts PAPS to APS and KO K00395 converts APS to sulfite in dissimilatory sulfate reduction. Figure generated from KEGG database website.

FIG. 12A shows the univariate comparison of the relative abundance (after log₁₀ transformation) of gut microbiome genes/KO K03190: urease accessory protein, that changed significantly after MTT in ASD but did not become similar to TD. Dashed lines represent the median of donors. Lower-dashed lines represent the mean of maintenance (n=2) and upper represent the median of major donors (n=5). Dark colored dots represent ASD individuals and light grey-colored dots represent TD. Asterisks represent significant differences between ASD Baseline, and the other groups (*Single asterisk indicates p<0.05, **double asterisks indicate p<0.01, triple *** asterisks indicate p<0.001, ns not significant, all p-values are FDR corrected). ASD: Autism Spectrum Disorders, TD: Typically Developing.

FIG. 12B shows the univariate comparison of the relative abundance (after log₁₀ transformation) of gut microbiome genes/KO K08717: Urea transporter, that changed significantly after MTT in ASD but did not become similar to TD. Dashed lines represent the median of donors. Lower-dashed lines represent the mean of maintenance (n=2) and upper represent the median of major donors (n=5). Dark colored dots represent ASD individuals and light grey-colored dots represent TD. Asterisks represent significant differences between ASD Baseline, and the other groups (*Single asterisk indicates p<0.05, **double asterisks indicate p<0.01, triple *** asterisks indicate p<0.001, ns not significant, all p-values are FDR corrected). ASD: Autism Spectrum Disorders, TD: Typically Developing.

FIG. 12C shows the univariate comparison of the relative abundance (after log₁₀ transformation) of gut microbiome genes/KO K13990: Urea transporter, that changed significantly after MTT in ASD but did not become similar to TD. Dashed lines represent the median of donors. Upper-dashed lines represent the mean of maintenance (n=2) and lower represent the median of major donors (n=5). Dark colored dots represent ASD individuals and light grey-colored dots represent TD. Asterisks represent significant differences between ASD Baseline, and the other groups (*Single asterisk indicates p<0.05, **double asterisks indicate p<0.01, triple *** asterisks indicate p<0.001, ns not significant, all p-values are FDR corrected). ASD: Autism Spectrum Disorders, TD: Typically Developing.

FIG. 12D shows the univariate comparison of the relative abundance (after log₁₀ transformation) of gut microbiome genes/KO K01438: acetylornithine deacetylase, that changed significantly after MTT in ASD but did not become similar to TD. Dashed lines represent the median of donors. Lower-dashed lines represent the mean of maintenance (n=2) and upper represent the median of major donors (n=5). Dark colored dots represent ASD individuals and light grey-colored dots represent TD. Asterisks represent significant differences between ASD Baseline, and the other groups (*Single asterisk indicates p<0.05, **double asterisks indicate p<0.01, triple *** asterisks indicate p<0.001, ns not significant, all p-values are FDR corrected). ASD: Autism Spectrum Disorders, TD: Typically Developing.

FIG. 13 shows univariate comparison of the relative abundance (after log₁₀ transformation) of genes encoding for enzymes involving microbial sulfur metabolism (dissimilatory sulfate reduction) before and after MTT in ASD in comparison with TD. (top left) K01082 (BPNT1/cycQ) 3′(2′), 5′-bisphosphate nucleotidase, (bottom left) K00395 (aprB) adenylylsulfate reductase, subunit B. (right) is a diagram Illustrating dissimilatory sulfur reduction and the contribution of BPNT1 and aprB to the process. Each dark colored dot represents an ASD individual and light grey-colored dots represent TD. Lower-dashed lines represent the mean of maintenance (n=2) and upper represents the median of major donors (n=5) in the top left graph. In the bottom left graph upper-dashed lines represent the mean of maintenance (n=2) and lower represent the median of major donors (n=5). Asterisks represent significant differences between ASD Baseline and the other groups (* single asterisk indicates p<0.05; ** double asterisks indicate p<0.01; ns: not significant; all p-values are FDR-corrected). ASD: autism spectrum disorder; TD: typically developing.

FIG. 14 shows a subset of correlation network between microbiome and pathways with plasma metabolites. Selenomonas sp.-oral-taxon-136 negatively correlated (R<−0.6, adjusted p<0.05) with NAD+ producing KOs K00330, K00337, K00339, K00340 and dissimilatory sulfur metabolism associated KO K01082. Darker lines (between Streptomyces autolyticus, Butyrivibrio proteoclasticus, Neisseria meningitidis and Actinobacteria connecting Hippurate) represent positive and lighter lines negative correlations.

FIG. 15A shows correlation tests between taxa and microbial genes between relative abundance of K01082:3′ (2′),5′-biphosphate nucleotidase and Selenomonas species,

FIG. 15B shows correlation tests between taxa and microbial genes between relative abundance of K01082: 3′ (2′),5′-biphosphate nucleotidase and Desulfovibrio piger.

FIG. 16A shows univariate comparison of the relative abundance (after log 10 transformation) of Nostoc linckia species before and after MTT. Upper lines for maintenance (n=2) and lower for major (n=5) donors. Each dark colored dot represents one ASD individual and light grey colored dots represent TD (typically developing). Asterisks represent significant differences between ASD Baseline, and the other groups (*Single asterisk indicates p<0.05, **double asterisks indicate p<0.01, ns not significant, all p-values are FDR corrected).

FIG. 16B shows univariate comparison of the relative abundance (after log 10 transformation) of Selenomonas sp. oral taxon-136 species before and after MTT. Upper lines for maintenance (n=2) and for major (n=5) donors. Each dark colored dot represents one ASD individual and light grey colored dots represent TD (typically developing). Asterisks represent significant differences between ASD Baseline, and the other groups (*Single asterisk indicates p<0.05, **double asterisks indicate p<0.01, ns not significant, all p-values are FDR corrected).

FIG. 17 is an overview illustration of microbiome and metabolic pathways/KOs in children with ASD before and after MTT. Gastrointestinal Symptom Rating Scale (GSRS); Childhood Autism Rating Scale (CARS).

### DETAILED DESCRIPTION

Unless defined otherwise, all technical and scientific terms used herein have the same meaning as commonly understood by one of ordinary skill in the art to which this disclosure belongs.

All publications, patents, and patent applications mentioned in this specification are herein incorporated by reference to the same extent as if each individual publication, patent, or patent application was specifically and individually indicated to be incorporated by reference.

As used herein and in the appended claims, the singular forms “a,” “an,” and “the” include plural referents unless the context clearly dictates otherwise. As used herein, the term “substantially” as in, for example, the phrase “substantially all peptides of an array,” refers to at least 90%, preferably at least 95%, more preferably at least 99%, and most preferably at least 99.9%, of the peptides of an array. Other uses of the term “substantially” involve an analogous definition.

Where a range of values is provided, it is understood that each intervening value, between the upper and lower limit of that range and any other stated or intervening value in that stated range is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding both of those included limits are also included in the disclosure.

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures. In the context of autism spectrum disorder, “treat” and “treating” encompass alleviating, ameliorating, delaying the onset of, inhibiting the progression of, or reducing the severity of one or more symptoms associated with an autism spectrum disorder.

As used herein, a “subject” can be a human or animal including, but not limited to, a dog, cat, horse, cow, pig, sheep, goat, chicken, rodent, e.g., rats and mice, and primate, e.g., monkey. Preferred subjects are human subjects. The human subject may be a pediatric, adult or a geriatric subject.

As used herein, a “microbiota” and “flora” refer to a community of microbes that live in or on a subject's body, both sustainably and transiently, including eukaryotes, archaea, bacteria, and viruses (including bacterial viruses (i.e., phage)). A “fecal microbiota” or “fecal microbiota preparation” refers to a community of microbes present in or prepared from a subject's feces. A non-selective fecal microbiota refers to a community or mixture of fecal microbes derived from a donor's fecal sample without selection and substantially resembling microbial constituents and population structure found in such fecal sample.

As used herein, “therapeutically effective amount” or “pharmaceutically active dose” refers to an amount of a composition which is effective in treating the named disease, disorder or condition.

As used herein, “isolated” or “purified” refers to a bacterium or other entity or substance that has been (1) separated from at least some of the components with which it was associated when initially produced (whether in nature or in an experimental setting), and/or (2) produced, prepared, purified, and/or manufactured by the hand of man. Isolated or purified bacteria can be separated from at least about 10%, about 20%, about 30%, about 40%, about 50%, about 60%, about 70%, about 80%, about 90%, or more of the other components with which they were initially associated.

As used herein, the terms “non-pathogenic” in reference to a bacterium or any other organism or entity includes any such organism or entity that is not capable of causing or affecting a disease, disorder or condition of a host organism containing the organism or entity.

As used herein, “spore” or a population of “spores” includes bacteria (or other single-celled organisms) that are generally viable, more resistant to environmental influences such as heat and bactericidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

As used herein, “colony forming units” (cfu) refers to an estimate of the number of viable microorganism cells in a given sample. The number of cfu can be assessed by counting the number of colonies on an agar plate as in standard methods for determining the number of viable bacterial cells in a sample.

As used herein, “viable” means possessing the ability to multiply. The viability of bacterial populations can be monitored as a function of the membrane integrity of the cell. Cells with a compromised membrane are considered to be dead or dying, whereas cells with an intact membrane are considered live. For example, SYTO 9 and propidium iodide are used to stain and differentiate live and dead bacteria. See Stocks, Cytometry A. 2004 October; 61(2):189-95. Cell viability can also be evaluated via molecular viability analyses, e.g., a PCR-based approach, which can differentiate nucleic acids associated with viable cells from those associated with inactivated cells. See Cangelosi and Mescheke, Appl Environ Microbiol. 2014 October; 80(19): 5884-5891.

As used herein, “Shannon Diversity Index” refers to a diversity index that accounts for abundance and evenness of species present in a given community using the formula H=−Σᵢ₌₁^{R}pᵢ ln pᵢ, where H is Shannon Diversity Index, R is the total number of species in the community, and pᵢ is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117 pp. As used herein, “antibiotic” refers to a substance that is used to treat and/or prevent bacterial infection by killing bacteria, inhibiting the growth of bacteria, or reducing the viability of bacteria.

Autism spectrum disorder (ASD) is a neurodevelopmental disorder that is characterized by impairments in social interaction and communication, restricted interests, and repetitive behavior. Individuals on the autism spectrum experience widely varying degrees and types of impairments, from mild to severe. Although early detection and interventions are encouraged to maximize the benefits and reduce the severity of the symptoms, individuals of any age can benefit from interventions and therapies that can reduce symptoms and increase skills and abilities. Appropriate subjects for the methods described herein include, without limitation, humans diagnosed as having or suspected of having autism spectrum disorder. In some cases, appropriate subjects for the methods provided herein are considered to be at increased risk (e.g., moderate or high risk) of developing ASD. In some cases, the subject has been diagnosed as having a condition meeting diagnostic criteria for ASD as set forth in the DSM-V. In other cases, the subject has a well-established DSM-IV diagnosis of autistic disorder, Asperger's disorder, or pervasive developmental disorder not otherwise specified (PDD-NOS).

The methods provided herein result in, or are aimed at achieving a detectable improvement in one or more indicators or symptoms of ASD including, without limitation, including, but not limited to, changes in eye tracking, skin conductance and/or electroencephalogram (EEG) measurements in response to visual stimuli, difficulties engaging in and responding to social interaction, verbal and nonverbal communication problems, repetitive behaviors, intellectual disability, difficulties in motor coordination, attention issues, sleep disturbances, and physical health issues such as gastrointestinal disturbances.

Several screening instruments are known in the art for evaluating a subject's social and communicative development and thus can be used as aids in screening for and detecting changes in the severity of impairment in communication skills, social interactions, and restricted, repetitive and stereotyped patterns of behavior characteristic of autism spectrum disorder. Evaluation can include neurologic and genetic assessment, along with in-depth cognitive and language testing. Additional measures developed specifically for diagnosing and assessing autism include the Autism Diagnosis Interview-Revised (ADI-R), the Autism Diagnostic Observation Schedule (ADOS-G) and the Childhood Autism Rating Scale (CARS).

According to CARS, evaluators rate the subject on a scale from 1 to 4 in each of 15 areas: Relating to People; Imitation; Emotional Response; Body Use; Object Use; Adaptation to Change; Visual Response; Listening Response; Taste, Smell, and Touch Response and Use; Fear; Verbal Communication; Nonverbal Communication; Activity; Level and Consistency of Intellectual Response; and General Impressions.

A second edition of CARS, known as the Childhood Autism Rating Scale-2 or CARS-2, was developed by Schopler et al. (Childhood Autism Rating Scale-Second edition (CARS2): Manual. Los Angeles: Western Psychological Services, 2010). The original CARS was developed primarily with individuals with co-morbid intellectual functioning and was criticized for not accurately identifying higher functioning individuals with ASD. CARS-2 retained the original CARS form for use with younger or lower functioning individuals (now renamed the CARS2-ST for “Standard Form”), but also includes a separate rating scale for use with higher functioning individuals (named the CARS2-HF for “High Functioning”) and an unscored information-gathering scale (“Questionnaire for Parents or Caregivers” or CARS2-QPC) that has utility for making CARS2ST and CARS2-HF ratings.

Another symptom rating instrument useful for assessing changes in symptom severity before, during, or following treatment according to a method provided herein is the Aberrant Behavior Checklist (ABC). See Aman et al., Psychometric characteristics of the aberrant behavior checklist. Am J Ment Defic. 1985 March; 89(5):492-502. The ABC is a symptom rating checklist used to assess and classify problem behaviors of children and adults in a variety of settings. The ABC includes 58 items that resolve onto five subscales: (1) irritability/agitation, (2) lethargy/social withdrawal, (3) stereotypic behavior, (4) hyperactivity/noncompliance, and (5) inappropriate speech.

The present inventors observed that autistic individuals, regardless of the presence or absence of comorbid gastrointestinal distress, have fewer species of gut bacteria as compared to neurotypical individuals. The present inventors also found that restoring the species diversity of gut bacteria helps to treat autistic symptoms in patients in need thereof. The inventors utilized shotgun metagenomic sequencing to determine the frequency of metabolic pathway genes in the gut microbiome of the treated individuals. These studies found that glutamate and sulfur metabolism pathway gene abundance decreased, while folate biosynthesis and oxidative stress protection pathway gene abundance increased following microbiota transport therapy (MTT). The present inventors also found significant increases in the genus Prevotella, Bifidobacterium and Desulfovibrio after MTT. Interestingly, the present inventors also found an increase in the genus of sulfur reducing bacteria, Selenomonas, which was negatively correlated with the relative abundance of sulfur reducing genes. In one aspect, the current disclosure encompasses the use of shotgun metagenomic sequencing to indicate the difference between the baseline abundance level and the abundance level after the treatment period of the one or more gut microbes specific metabolic genes in the gut microbiome. Interestingly, the current disclosure shows that shot gun metagenomic methods can be successfully used to determine the efficacy of treatment following MTT.

In one aspect, this application provides a method for treating an autism spectrum disorder (ASD) in a subject in need thereof, the method comprising administering to the subject an amount of a pharmaceutical composition effective for treating the ASD, where the pharmaceutical composition comprises a fecal microbe preparation, where the subject exhibits at least a 10% reduction in ASD symptom severity after the treatment as compared to before initiating the treatment. In one aspect, ASD symptom severity is assessed by Childhood Autism Rating Scale (CARS). In another aspect, ASD symptom severity is assessed by Childhood Autism Rating Scale 2-Standard Form (CARS2-ST). In a further aspect, ASD symptom severity is assessed by Childhood Autism Rating Scale 2-High Functioning (CARS2-HIF). In one aspect, ASD symptom severity is assessed by Aberrant Behavior Checklist (ABC). In another aspect, ASD symptom severity is assessed by Social Responsiveness Scale (SRS). In another aspect, ASD symptom severity is assessed by Vineland Adaptive Behavior Scale II (VABS-II). In one aspect, a treatment results in an improvement of at least 10%, 20%, 30%, 40%, 50%, 60%, 70%, 80%, or 90% based on the Leiter International Performance Scale (see Roid, G. H., & Miller, L. J. (1997). Leiter International Performance Scale—Revised. Wood Dale, IL: Stoelting) in an ASD patient. In another aspect, an ASD symptom severity improvement is measured using any one of the foregoing assessment systems after at least 8, 16, 24, 32, 40, 50, 52, 54, 60, 70, 80, 90, 100, 102, 104, 106, 108, 110, 112, 118, 120, 124, 130, 132, 140, 148, or 150 weeks of treatment and compared to a measurement prior to the treatment.

The present disclosure also discloses a method of maintaining a reduced severity of an autism spectrum disorder in a human subject, comprising: (a) administering a non-absorbable antibiotic to an autistic human subject; (b) subjecting the autistic human subject to a bowel cleanse; and (c) administering purified fecal microbiota from a neurotypical human donor to the human subject; wherein the human subject exhibits a significant improvement in symptom severity based on an assessment system selected from the group consisting of CARS, CARS2-ST, CARS2-HF, ABC, SRS, and VABS-II. These improvements in symptoms are found to correlate well with the abundance of certain genes characteristic of specific microbes and differential microbial pathways, as evident from shotgun metagenomic analysis of treated autistic human subjects.

One of ordinary skill in the art understands that the foregoing assessment systems are only exemplary tools for evaluating ASD-related social and cognitive symptoms. Other similar tools can be used or designed to evaluate core ASD-related symptoms. Additional assessment systems are described in U.S. Patent Application Publication No. US2019/0358274, which is incorporated here in its entirety. For example, in one aspect, a treatment results in an improvement of at least 10%, 20%, 30%, 40%, 50%, 60%, 70%, 80%, or 90% based on Autism Treatment Evaluation Checklist (ATEC). See Rimland and Edelson: Autism Treatment Evaluation Checklist: Statistical Analyses. Autism Research Institute 2000. In another aspect, a treatment results in an improvement of at least 10%, 20%, 30%, 40%, 50%, 60%, 70%, 80%, or 90% based on Pervasive Developmental Disorders Behavior Inventory (PDD-BI). See Cohen et al., The PDD Behavior Inventory: a rating scale for assessing response to intervention in children with pervasive developmental disorder. J Autism Dev Disord. 2003 33(1):31-45. In yet another aspect, a treatment results in an improvement of at least 10%, 20%, 30%, 40%, 50%, 60%, 70%, 80%, or 90% based on Severity of Autism Scale (SAS). See Adams et al., The severity of autism is associated with toxic metal body burden and red blood cell glutathione levels. J Toxicol. 2009, 2009:532640. In a further aspect, an improvement of autism-related symptoms or an symptom severity reduction is assessed based on any one of the system or scale mentioned in Aman et al., Outcome Measures for Clinical Drug Trials in Autism, CNS Spectr. 9(1): 36-47 (2004). In a further aspect, an improvement of autism-related symptoms or an symptom severity reduction is assessed based on any one of the symptom characterization systems listed in Table A. In one aspect, a symptom improvement over any one of the foregoing systems is measured after at least 2, 4, 6, 8, 16, 24, 32, 40, 50, 52, 54, 60, 80, 90, 92, 94, 96, 98, 100, 102, 104, 106, 108, 112, 118, 124, 132, 140, 148 or 150 weeks of treatment or after treatment and compared to a measurement prior to the treatment. In one aspect, a symptom improvement over any of the foregoing systems is measured after at least 2 weeks of treatment or after treatment and compared to a measurement prior to the treatment. In one aspect, a symptom improvement over any one of the foregoing systems is measured after discontinuing treatment for at least 2, 4, 6, 8, 10, 16, 24, 32, 40, 50, 52, 54, 60, 80, 90, 100, 110, 120 or more weeks and compared to a measurement prior to the treatment. In one aspect, an ASD symptom improvement of 10% after at least 8 weeks is maintained for between 50 and 120 weeks after the treatment as compared to before initiating the treatment.

TABLE A
Selected outcome measures that can be used to monitor core ASD-related social and cognitive symptoms.
Validated Outcome Measures

| | Description | |
|---|---|---|
| Tool | Autism Symptoms | Rater |
| ADOS | The Autism Diagnostic Observation Schedule (ADOS) is a gold standards | Trained |
| | instrument for diagnosing ASD with the largest evidence base and highest | Examiner |
| | sensitivity and specificity | |
| OACIS | The Ohio Autism Clinical Impression Scale was developed to be sensitive to | Clinician |
| | subtle, but clinically-meaningful changes in core and associated ASD symptoms | |
| | using a focused scaling system that assesses severity and improvement in ASD | |
| | behaviors similar to the widely used Clinical Global Impression Scale. | |
| SRS | The Social Responsiveness Scale is a standardized and validated quantitative | Parent or |
| | scale that measures the severity and type of social impairments that are | Teacher |
| | characteristic of ASD | |
| SCQ | Social Communication Questionnaire is brief instrument that evaluates | Parent or |
| | communication skills and social functioning. Both the current and lifetime | Teacher |
| | editions will be used as appropriate | |
| AIM | The Autism Impact Measure is a recently developed parent-report measure that | Parent |
| | assesses both frequency and impact of current core ASD symptoms during the past | |
| | 2-weeks. Initial studies have demonstrated excellent psychometric properties and | |
| | construct validity | |
| | Behavior | |
| ABC | The Aberrant Behavior Checklist is a validated questionnaire that rates symptoms | Parent or |
| | of hyperactivity, irritability, lethargy, and stereotypic behavior in individuals with | Teacher |
| | developmental disabilities. It has been used in multiple clinical trials in ASD and | |
| | has convergent and divergent validity | |
| CBCL | Child Behavior Checklist is an easy to complete standardized questionnaire that | Parent or |
| | assesses a wide range of behaviors associated with ASD symptoms, including | Teacher |
| | anxiety, depression, withdraw, sleep problems, somatic problems, and aggressive | |
| | and destructive behavior | |
| BASC | The Behavioral Assessment System for Children provides scales of cognition | Teacher |
| | function, behavior, social function, and academic problems. This scale measures a | Parent or |
| | wide range of behaviors including hyperactivity, attention, depression, anxiety, | |
| | and executive function. | |
| | Language | |
| CELF | The Clinical Evaluations of Language Fundamentals is one of the only | Trained |
| | standardized, well-validated language assessment instruments that spans the age | Examiner |
| | range of most participants (using both CELF-preschool-2 and CELF-4). It | |
| | assesses a wide range of language skills that are only partially measured by other | |
| | language tests, including high-level language skills that are abnormal in | |
| | individuals with ASD, such as language pragmatics and has been used in several | |
| | recent studies focusing on core language deficits in ASD | |
| PLS | The Preschool Language Scale-4 is used in conjunction with the CELF since it is | Trained |
| | also a standardized, well-validated language assessment instrument and can | Examiner |
| | measure subtle changes in language in children with poor language abilities | |
| | Adaptive Behavior | |
| VABS | The Vineland Adaptive Behavior Scale is a widely used standardized, well- | Trained |
| | validated assessment tool for children with developmental delays that measures | Interviewer |
| | functional abilities within several domains. It is particularly useful for children | |
| | with intellectual disability which commonly co-occurs with ASD and has valid | |
| | measures of social impairments in children with ASD | |
| | Intellect | |
| Leiter-R | The Leiter-R, due to its non-verbal nature, is an excellent unbiased measure of | Trained |
| | intellect when language impairment exists. It assesses a wide range of ages (2-21 | Examiner |
| | years) and contains attention and memory batteries which are skills often | |
| | disrupted in ASD. The Leiter-R is designed to measure growth in all domains it | |
| | assesses, making it sensitive to change due to treatment. Studies have shown good | |
| | psychometric properties and verified that it is generally recommended for use in | |
| | children with ASD | |
| WISC/WPPSI | The Wechsler Intelligence Scale for Children is one of the oldest and most widely | Trained |
| | used tests of intelligence for children. For children younger than 6 years the | Examiner |
| | Wechsler Preschool and Primary Scale of Intelligence test is used. One | |
| | disadvantage when using this with children with ASD is its reliance on language. | |

In an aspect, a treatment method effects a cure, reduction of the symptoms, or a percentage reduction of symptoms of a disorder (e.g., ASD). The change of flora is preferably as “near-complete” as possible and the flora is replaced by viable organisms which will crowd out any remaining, original flora. Typically, the change in enteric flora comprises introduction of an array of predetermined flora into the gastro-intestinal system, and thus in a preferred form the method of treatment comprises substantially or completely displacing pathogenic enteric flora in patients requiring such treatment.

In one aspect, a treatment achieves at least 10%, 20%, 30%, 40%, 50%, 60%, 70%, 80%, or 90% reduction in ASD symptom severity after 2 or more weeks, 4 or more weeks, 6 or more weeks, or 8 or more weeks of treatment as compared to before initiating the treatment, where the ASD symptom severity is assessed by a method selected from the group consisting of CARS, CARS2-ST, CARS2-HIF, ABC, SRS, and VABS-II.

In another aspect, a treatment achieves between 10% and 20%, between 10% and 30%, between 10% and 40%, between 10% and 50%, between 10% and 60%, between 10% and 70%, between 10% and 80%, between 10% and 90%, between 20% and 30%, between 20% and 40%, between 20% and 50%, between 20% and 60%, between 20% and 70%, between 20% and 80%, between 20% and 90%, between 30% and 40%, between 30% and 50%, between 30% and 60%, between 30% and 70%, between 30% and 80%, between 30% and 90%, between 40% and 50%, between 40% and 60%, between 40% and 70%, between 40% and 80%, between 40% and 90%, between 50% and 60%, between 50% and 70%, between 50% and 80%, or between 50% and 90% reduction in ASD symptom severity after 2 or more weeks, 4 or more weeks, 6 or more weeks, 8 or more weeks, 12 or more weeks, 16 or more weeks, or 18 or more weeks of treatment as compared to before initiating the treatment, where the ASD symptom severity is assessed by a method selected from the group consisting of CARS, CARS2-ST, CARS2-HIF, ABC, SRS, and VABS-II.

In one aspect, a treatment achieves at least 10%, 20%, 30%, 40%, 50%, 60%, 70%, 80%, or 90% reduction in ASD symptom severity and substantially maintains the symptom severity reduction for at least 8, 12, 16, 20, 24, or 28 weeks after discontinuing the treatment, where the ASD symptom severity is assessed by CARS, CARS2-ST, CARS2-H1F, ABC, SRS, or VABS-II.

In one aspect, an ASD subject being treated exhibits no gastrointestinal (GI) symptom prior to initiating a treatment. In another aspect, an ASD subject being treated exhibits one or more GI symptoms prior to initiating a treatment. In one aspect, an ASD subject being treated exhibits at least a 10%, 20%, 30%, 40%, 50%, 60%, 70%, 80%, or 90% reduction in GI symptom severity after a treatment as compared to before initiating the treatment. In one aspect, GI symptom severity is assessed by the Gastrointestinal Symptom Rating Scale (GSRS). In another aspect, a treatment achieves between 10% and 20%, between 20% and 30%, between 20% and 40%, between 20% and 50%, between 20% and 60%, between 20% and 70%, between 20% and 80%, between 20% and 90%, between 30% and 40%, between 30% and 50%, between 30% and 60%, between 30% and 70%, between 30% and 80%, between 30% and 90%, between 40% and 50%, between 40% and 60%, between 40% and 70%, between 40% and 80%, between 40% and 90%, between 50% and 60%, between 50% and 70%, between 50% and 80%, or between 50% and 90% reduction in GI symptom severity in an ASD patient after 8 or more weeks of treatment as compared to before initiating the treatment, where the GI symptom severity is assessed by GSRS.

In one aspect, a symptom severity reduction (e.g., for ASD symptoms, GI symptoms, or both) is ongoing during a treatment or sustained after finishing or discontinuing a treatment. In one aspect, a symptom severity reduction (e.g., for ASD symptoms, GI symptoms, or both) is assessed at a specific time point during or post treatment, e.g., about 2, 4, 6, 8, 12, 18, 24, 32, 40, 48, 50, 52, 54, 60, 70, 80, 90, 92, 94, 96, 98, 100, 102, 104, 106, 108, 112, 118, 124, 132, 140, 148 weeks after initiating a treatment, or about 2, 4, 6, 8, 12, 18, 24, 32, 40, 48, 50, 52, 54, 60, 70, 80, 90, 92, 94, 96, 98, 100, 102, 104, 106, 108, 112, 118, 124, 132, 140, 148 weeks after finishing or discontinuing a treatment. In one aspect, a subject maintains an increased abundance of one or more gut microorganisms after initiating the treatment. In another aspect, an increase in abundance of one or more gut microorganisms is at least a 2-fold, 3-fold, 4-fold, 5-fold, 10-fold, 15-fold, 20-fold, 25-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, or 900-fold increase. In another aspect, an increase in abundance of one or more gut microorganisms is between 2- and 5-fold, 5- and 10-fold, 15- and 30-fold, 30- and 40-fold, 40- and 50-fold, 50- and 60-fold, 60- and 70-fold, 70- and 80-fold, 80- and 90-fold, 90- and 100-fold, 100- and 200-fold, 200- and 300-fold, 300- and 400-fold, 400- and 500-fold, 500 and 600-fold, 600- and 700-fold, 700- and 800-fold, or 900- and 950-fold increase. In yet another aspect, the increase in abundance of one or more gut microorganisms is maintained for least one year after completing the treatment. In yet another aspect, the increase in abundance of one or more gut microorganisms is maintained for least two years after completing the treatment. In another aspect, the increase in abundance of one or more gut microorganisms is maintained for at least 8, 16, 24, 32, 40, 50, 52, 54, 60, 80, 90, 92, 94, 96, 98, 100, 102, 104, 106, 108, 112, 118, 124, 132, 140, 148 or 150 weeks after completing the treatment. In a further aspect, the increase in abundance of one or more gut microorganisms is maintained from 8 to 16, 16 to 24, 24 to 32, 32 to 40, 40 to 50, 50 to 60, 60 to 70, 70 to 80, 80 to 90, 90 to 96, 96 to 100, 100 to 108, 108 to 112, 112 to 124, 124 to 132. 132 to 148, or 148 to 150 weeks after completing the treatment. In another aspect, a subject maintains an increased abundance of two or more, three or more, four or more, five or more, six or more, or seven or more gut microorganisms.

In one aspect, a subject maintains an increased abundance of one or more gut microorganisms after initiating the treatment. In another aspect an increase in abundance of one or more microbes comprises at least one species selected belonging to a genus selected from Prevotella, Bifidobacterium, and Desulfovibrio. In another aspect, an increase in abundance of one or more gut microorganisms is at least a 2-fold, 3-fold, 4-fold, 5-fold, 10-fold, 15-fold, 20-fold, 25-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, or 900-fold increase. In another aspect, an increase in abundance of one or more gut microorganisms is between 2- and 5-fold, 5- and 10-fold, 15- and 30-fold, 30- and 40-fold, 40- and 50-fold, 50- and 60-fold, 60- and 70-fold, 70- and 80-fold, 80- and 90-fold, 90- and 100-fold, 100- and 200-fold, 200- and 300-fold, 300- and 400-fold, 400- and 500-fold, 500 and 600-fold, 600- and 700-fold, 700- and 800-fold, or 900- and 950-fold increase. In yet another aspect, the increase in abundance of one or more gut microorganisms is maintained for least one year after completing the treatment. In yet another aspect, the increase in abundance of one or more gut microorganisms is maintained for least two years after completing the treatment. In another aspect, the increase in abundance of one or more gut microorganisms is maintained for at least 8, 16, 24, 32, 40, 50, 52, 54, 60, 80, 90, 92, 94, 96, 98, 100, 102, 104, 106, 108, 112, 118, 124, 132, 140, 148 or 150 weeks after completing the treatment. In a further aspect, the increase in abundance of one or more gut microorganisms is maintained from 8 to 16, 16 to 24, 24 to 32, 32 to 40, 40 to 50, 50 to 60, 60 to 70, 70 to 80, 80 to 90, 90 to 96, 96 to 100, 100 to 108, 108 to 112, 112 to 124, 124 to 132. 132 to 148, or 148 to 150 weeks after completing the treatment. In another aspect, a subject maintains an increased abundance of two or more, three or more, four or more, five or more, six or more, or seven or more gut microorganisms.

In one aspect, a subject maintains an alteration in gut microbiome metabolic pathways after initiating the treatment. In another aspect, the alteration in gut microbiome metabolic pathways comprises an increase in folate biosynthesis and/or oxidative stress protection pathways, or a decrease in glutamate and/or sulfur metabolism pathways, or both. In another aspect the alteration in gut microbiome metabolic pathways is between an about 0.5-fold and about 2-fold change. In another aspect the alteration in gut microbiome metabolic pathways is between an about 1-fold and about 2-fold change. In another aspect the alteration in gut microbiome metabolic pathways is about a 2-fold change. In another aspect the alteration in gut microbiome metabolic pathways is at most a 2-fold change. In another aspect the alteration in gut microbiome metabolic pathways is a significant fold change. In another aspect the alteration in gut microbiome metabolic pathways is at least a 0.5-fold, 1-fold, 2-fold, 3-fold, 4-fold, 5-fold, 10-fold, 15-fold, 20-fold, 25-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, or 900-fold increase. In another aspect the alteration in gut microbiome metabolic pathways is between an about 0.5-fold and about 2-fold increase. In another aspect the alteration in gut microbiome metabolic pathways is at least a 0.5-fold, 1-fold, 2-fold, 3-fold, 4-fold, 5-fold, 10-fold, 15-fold, 20-fold, 25-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, or 900-fold decrease. In another aspect the alteration in gut microbiome metabolic pathways is between an about 0.5-fold and about 2-fold decrease. In another aspect the alteration in gut microbiome metabolic pathways is both at least a 0.5 fold, 1-fold, 2-fold, 3-fold, 4-fold, 5-fold, 10-fold, 15-fold, 20-fold, 25-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, or 900-fold increase and/or decrease. In yet another aspect, the alteration in gut microbiome metabolic pathways is maintained for least one year after completing the treatment. In yet another aspect, the alteration in gut microbiome metabolic pathways is maintained for least two years after completing the treatment. In another aspect, the alteration in gut microbiome metabolic pathways is maintained for at least 8, 16, 24, 32, 40, 50, 52, 54, 60, 80, 90, 92, 94, 96, 98, 100, 102, 104, 106, 108, 112, 118, 124, 132, 140, 148 or 150 weeks after completing the treatment. In a further aspect, the alteration in gut microbiome metabolic pathways is maintained from 8 to 16, 16 to 24, 24 to 32, 32 to 40, 40 to 50, 50 to 60, 60 to 70, 70 to 80, 80 to 90, 90 to 96, 96 to 100, 100 to 108, 108 to 112, 112 to 124, 124 to 132. 132 to 148, or 148 to 150 weeks after completing the treatment.

In one aspect, the difference between the baseline relative abundance level and the relative abundance level after the treatment period of one or more gut microbes is measured using sequencing. In one aspect, the difference between the baseline relative abundance level and the relative abundance level after the treatment period of the one or more gut microbes is measured using amplicon sequencing. In one aspect, the difference between the baseline relative abundance level and the relative abundance level after the treatment period of the one or more gut microbes is measured using amplicon sequencing of the 16S ribosomal RNA (rRNA) gene. In one aspect, the difference between the baseline relative abundance level and the relative abundance level after the treatment period of the one or more gut microbes is measured using shotgun metagenomic sequencing. In one aspect, the increased relative abundance of one or more gut microorganisms after initiating the treatment is determined using shotgun metagenomic sequencing of the subject's gut microbiome with paired ends of 150 bp and minimum 10 million reads on Illumina NextSeq 500 platform. Microbiome relative abundance was calculated with Kraken2 (v2.0.7) with NCBI RefSeq database (Wood et al., 2019) and Bracken (Bayesian Re-estimation of Abundance with KrakEN, v2.6) (Lu et al. 2017).

In one aspect, the alteration in gut microbiome metabolic pathways is determined via metabolic profiling of the subject's gut microbiome. HUMAnN2 (the HMP unified metabolic analysis network, v2) was used to identify the functional genes/metabolic pathways associated with microbiome from ChocoPhlAn pangenome database, and the MetCyc, MinPath & UniRef90 databases (Truong et al., 2015).

In one aspect, a difference in relative abundance level between the baseline relative abundance level and the relative abundance level after the treatment period of one or more gut microbes and/or alternations of the metabolic pathways is determined via an assay selected from the group consisting of qPCR, RT-qPCR, clone libraries, DGGE, T-RFLP, ARISA, microarrays, FISH, dot-blot hybridization, a DNA hybridization method, and sequencing. In another aspect, a difference between the baseline relative abundance level and the relative abundance level after the treatment period of the one or more gut microbes is determined via 16S rDNA-targeted pyrosequencing. In a further aspect, a difference between the baseline relative abundance level and the relative abundance level after the treatment period of the one or more gut microbes is determined via a DNA hybridization assay based on a 16S rDNA sequence. In one aspect, a difference between the baseline relative abundance level and the relative abundance level after the treatment period of the one or more gut microbes is determined via detecting one or more proteins or metabolites specific to one or more gut microorganisms. In another aspect, a difference between the baseline relative abundance level and the relative abundance level after the treatment period of the one or more gut microbes is determined via an assay selected from the group consisting of 2-Dimensional Gel Electrophoresis, 2-Diminsional Difference Gel Electrophoresis (2D-DIGE), MALDI TOF-MS, (2D-) LC-ESI-MS/MS, AQUA and lTRAQ. In one aspect, a difference between the baseline relative abundance level and the relative abundance level after the treatment period of the one or more gut microbes is determined via shotgun metagenomic sequencing.

One way in which shotgun metagenomic sequencing may be carried out involves DNA sequence reads being first analyzed with MultiQC (Ewels et al., 2016. Adapters and low-quality reads (length <50 bp or phred <30) may be removed. To avoid the human genome contamination, mapped all the reads against the UCSC Genome Browser's hg38 human genome reference database using Burrows-Wheeler Aligner (bwa) (Li & Durbin, 2010) and may discarded mapped reads. Unmapped (without human genome) may then will be used for downstream analyses. Kraken 2 (Wood et al., 2019, Improved metagenomic analysis with Kraken 2. Genome Biol 20, 257 (2019) may be employed to generate taxonomic profiles from the shotgun reads, while HUMAnN2 (HMP Unified Metabolic Analysis Network) (Hall et al. A novel Ruminococcus gnavus clade enriched in inflammatory bowel disease patients. Genome medicine 2017; 9: 103) may be used to determine the metabolic contributions within samples. The proves may also involve mapping of the metagenomic reads against Uniref orthologous gene family, MetCyc UniPathway, and KEGG.

In one aspect, a subject maintains an increased relative abundance of one or more gut microorganisms following MTT, wherein the one or more gut microorganisms comprises at least one of Prevotella dentalis, Prevotella enoeca, Prevotella oris, Prevotella meloninogenica, Prevotella denticola, Prevotella fusca, Prevotella intermedia, Prevotella ruminicola, Bifidobacterium bifidum, Bifidobacterium angulatum, Selenomonas sp., Selenomonas sp. oral taxon 136, Alistipes finegoldii, Lactobacillus vaginalis, Desulfovibrio sp. and Desulfovibrio piger compared to an relative abundance baseline, wherein the relative abundance baseline is a predetermined value or is determined from a control sample. In one aspect, the control sample may be a stool sample from the subject prior to the treatment, a stool sample from a healthy subject, or a stool sample from a subject with an autism spectrum disorder (ASD) who has not been administered the pharmaceutical composition.

In one aspect the increase in relative abundance of one or more of Prevotella dentalis, Prevotella enoeca, Prevotella oris, Prevotella meloninogenica, Prevotella denticola, Prevotella fusca, Prevotella intermedia, Prevotella ruminicola, Bifidobacterium bifidum, Bifidobacterium angulatum, Selenomonas sp., Selenomonas sp. oral taxon 136, Alistipes finegoldii, Lactobacillus vaginalis, Desulfovibrio sp. and Desulfovibrio piger is detectable by increase in relative abundance of certain genes, characteristic of these microbes, as evident from shotgun metagenomic analysis of stool samples from treated and untreated patients. In some aspects, the microbial gene abundance is close to that of the healthy donor after about 5 weeks to about 5 years after treatment. In some aspects, the microbial gene abundance is close to that of the healthy donor after about 5 to about 10 weeks, or about 10 weeks to about 3 months, or about 3 months to about 6 months, or about 6 months to about 9 months, or about 9 months to about 12 months, or about 12 months to about 15 months, or about 15 months to about 18 months, or about 18 months to about 21 months, or about 21 months to about 24 months, or about 24 months to about 27 moths, or about 27 months to about 30 months, or about 30 months to about 33 months, or about 33 months to about 36 months or more.

In one aspect, a subject's gut microbiome maintains a decrease in the abundance of genes involved in glutamate metabolism and/or sulfur metabolism in the subject's gut microbiome following MTT, wherein the decrease is in comparison to the baseline levels of the glutamate metabolism and/or sulfur metabolism genes. In one aspect, the decrease in abundance of genes involved in glutamate metabolism and/or sulfur metabolism in the subject's gut microbiome following MTT can be assessed by shot gun metagenomic analysis. In some aspects the decreases in abundance of the genes involved in glutamate metabolism and/or sulfur metabolism is evident after about 5 weeks to about 5 years after treatment. In some aspects, the microbial gene abundance is close to that of the healthy donor after about 5 to about 10 weeks, or about 10 weeks to about 3 months, or about 3 months to about 6 months, or about 6 months to about 9 months, or about 9 months to about 12 months, or about 12 months to about 15 months, or about 15 months to about 18 months, or about 18 months to about 21 months, or about 21 months to about 24 months, or about 24 months to about 27 moths, or about 27 months to about 30 months, or about 30 months to about 33 months, or about 33 months to about 36 months or more.

In one aspect, a subject's gut microbiome maintains an increase in the relative abundance of genes involved in folate biosynthesis and/or oxidative stress protection in the subject's gut microbiome following MTT, wherein the increase is in comparison to the baseline levels of folate biosynthesis and/or oxidative stress protection genes. In some aspects, the increase in the abundance of genes involved in folate biosynthesis and/or oxidative stress protection in the subject's gut microbiome following MTT can be assessed by shot gun metagenomic analysis. In some aspects the increase in abundance of the genes involved in folate biosynthesis and/or oxidative stress protection is evident after about 5 weeks to about 5 years after treatment. In some aspects, the microbial gene abundance is close to that of the healthy donor after about 5 to about 10 weeks, or about 10 weeks to about 3 months, or about 3 months to about 6 months, or about 6 months to about 9 months, or about 9 months to about 12 months, or about 12 months to about 15 months, or about 15 months to about 18 months, or about 18 months to about 21 months, or about 21 months to about 24 months, or about 24 months to about 27 moths, or about 27 months to about 30 months, or about 30 months to about 33 months, or about 33 months to about 36 months or more.

In another aspect, a subject's gut microbiome maintains both a decrease in the abundance of genes involved in glutamate metabolism and/or sulfur metabolism and an increase in the abundance of genes involved in folate biosynthesis and/or oxidative stress protection in the subject's gut microbiome following MTT wherein the decrease is in comparison to the baseline levels of the glutamate metabolism and/or sulfur metabolism genes and wherein the increase is in comparison to the baseline levels of folate biosynthesis and/or oxidative stress protection genes.

In one aspect, a subject's increased abundance of one or more gut microorganisms following MTT, wherein the one or more gut microorganisms comprises at least one of Prevotella dentalis, Prevotella enoeca, Prevotella oris, Prevotella meloninogenica, Bifidobacterium bifidum, Bifidobacterium angulatum, and Desulfovibrio piger, is indicative of successful treatment of an autism spectrum disorder (ASD) in the subject.

In another aspect, decreased abundance of genes involved in glutamate metabolism and/or sulfur metabolism in the subject's gut microbiome following MTT is indicative of successful treatment of an autism spectrum disorder (ASD) in the subject.

In another aspect, increased abundance of genes involved in folate biosynthesis and/or oxidative stress protection in the subject's gut microbiome following MTT, is indicative of successful treatment of an autism spectrum disorder (ASD) in the subject.

In another aspect, decreased abundance of genes involved in glutamate metabolism and/or sulfur metabolism and increased abundance of genes involved in folate biosynthesis and/or oxidative stress protection in the subject's gut microbiome following MTT, is indicative of successful treatment of an autism spectrum disorder (ASD) in the subject.

In one aspect, a subject's increased abundance of one or more gut microorganisms following MTT, wherein the one or more gut microorganisms comprises at least one of Prevotella dentalis, Prevotella enoeca, Prevotella oris, Prevotella meloninogenica, Bifidobacterium bifidum, Bifidobacterium angulatum, and Desulfovibrio piger, is maintained for at least two years after treatment has stopped. In one aspect, the increased abundance is determined from a comparison to an abundance baseline. In one aspect, the abundance baseline is a predetermined value, or a value determined from a control sample. In one aspect, the control sample may be a stool sample from the patient prior to the treatment, a stool sample from a healthy subject, or a stool sample from a subject with an autism spectrum disorder (ASD) who has not been administered the pharmaceutical composition.

In one aspect, decreased abundance of genes involved in glutamate metabolism and/or sulfur metabolism in the subject's gut microbiome following MTT is maintained for at least two years after the treatment has stopped, wherein the decrease is determined from a comparison to the baseline levels of the glutamate metabolism and/or sulfur metabolism genes.

In one aspect, increased abundance of genes involved in folate biosynthesis and/or oxidative stress protection in the subject's gut microbiome following MTT is maintained for at least two years after the treatment has stopped, wherein the increase is determined from a comparison to the baseline levels of folate biosynthesis and/or oxidative stress protection genes.

In another aspect, decreased abundance of genes involved in glutamate metabolism and/or sulfur metabolism and increased abundance of genes involved in folate biosynthesis and/or oxidative stress protection in the subject's gut microbiome following MTT is maintained for at least two years after the treatment has stopped, wherein the decrease is determined from a comparison to the baseline levels of the glutamate metabolism and/or sulfur metabolism genes, and wherein the increase is determined from a comparison to the baseline levels of folate biosynthesis and/or oxidative stress protection genes.

In another aspect, surprisingly, a subject's increased abundance of one or more gut microorganisms following MTT included species from the genus of sulfur reducing bacteria, Selenomonas, which may be negatively correlated with the abundance of sulfur reducing genes.

In another aspect, a pharmaceutical composition used herein comprises a non-selective and substantially complete fecal microbiota supplemented with one or more viable, non-pathogenic microorganisms (e.g., one or more bacterial isolates) selected from the group consisting of Selenomonas, Prevotella, Bifidobacterium, and Desulfovibrio. In another aspect, a pharmaceutical composition used herein comprises fecal microbiota supplemented with one or more viable, non-pathogenic microorganisms selected from the group consisting of Prevotella, Bifidobacterium and Clostridium. In another aspect, a pharmaceutical composition used herein comprises a synthetic fecal composition of predetermined flora. In another aspect, a pharmaceutical composition used herein comprises a predetermined flora comprises a preparation of viable flora in proportional content that resembles a normal healthy human fecal flora and comprises no antibiotic resistant populations. In another aspect, a pharmaceutical composition used herein is administered as a solid dosage form selected from the group consisting of capsule, tablet, powder, and granule. In another aspect, a pharmaceutical composition used herein is formulated as an acid resistant capsule.

In another aspect, provided herein is a method of treating an autism spectrum disorder in a human subject. In exemplary aspects, the method comprises or consists essentially of the following steps: administering an antibiotic to a human subject; subjecting the human subject to a bowel cleanse; and administering purified fecal microbiota to the human subject, wherein an autism spectrum disorder is treated in the human subject.

In exemplary aspects, treating ASD comprises alleviating, ameliorating, delaying the onset of, inhibiting the progression of, or reducing the severity of one or more, two or more, three or more, four or more, five or more, six or more, seven or more, eight or more symptoms characteristic of ASD. In one aspect, a treatment alleviates, ameliorates, delays the onset of, inhibits the progression of, or reduces the severity of one or more social and cognitive core ASD-related symptoms. In some aspects, the symptom(s) is selected from the group consisting of: (i) insistence on sameness or resistance to change; (ii) difficulty in expressing needs; (iii) repeating words or phrases in place of normal, responsive language; (iv) laughing, crying, showing distress for reasons not apparent to others; (v) prefers to be alone or aloof manner; (vi) tantrums; (vii) difficulty in mixing with others; (viii) may not want to cuddle or be cuddled; (ix) little or no eye contact; (x) unresponsive to normal teaching methods; (xi) sustained odd play; (xii) apparent over-sensitivity or under-sensitivity to pain; (xiii) little or no real fears of danger; (xiv) noticeable physical over-activity or extreme under-activity; (xv) uneven gross/fine motor skills; and/or (xvi) non-responsiveness to verbal cues. In some aspects, the symptom(s) is selected from the group consisting of compulsive behavior, ritualistic behavior, restricted behavior, stereotypy, sameness, or self-injury. The methods described here can lead to improvement of any combination of the foregoing symptoms.

Subjects appropriate for treatment according to a method provided herein may not present with or report gastrointestinal distress symptoms prior to initiating a method as provided herein. In some cases, for example, a human subject appropriate for treatment according to a method provided herein manifests no gastrointestinal symptoms prior to or at the time at which treatment is begun. In one aspect, an ASD subject treated herein exhibit one or more or two or more GI symptoms selected from the group consisting of abdominal pain, reflux, indigestion, irritable bowel syndrome, chronic persistent diarrhea, diarrhea, flatulence, constipation, and alternating constipation/diarrhea.

Regardless of the presence or absence of gastrointestinal distress symptoms, human subjects appropriate for the methods provided herein typically have significantly fewer species of gut bacteria before the method of treatment as compared to a neurotypical human. In some cases, the human subject to be treated by the method exhibits at least about 20%, 30%, 40%, 50%, 60%, 70%, 80%, or 90% fewer species of gut bacterial prior to administration of the purified fecal microbiota dosage as compared to a neurotypical human.

In one aspect, a fecal microbiota preparation used in a method described here comprises a donor's entire or substantially complete microbiota. In one aspect, a fecal microbiota preparation comprises a non-selective fecal microbiota. In another aspect, a fecal microbiota preparation comprises an isolated or purified population of live non-pathogenic fecal bacteria. In a further aspect, a fecal microbiota preparation comprises a non-selective and substantially complete fecal microbiota preparation from a single donor. In another aspect, a therapeutic composition used herein comprises a mixture of live, non-pathogenic, synthetic bacteria or live, non-pathogenic, purified or extracted, fecal microbiota.

In one aspect, the preparation of a fecal microbiota preparation involves a treatment selected from the group consisting of ethanol treatment, detergent treatment, heat treatment, irradiation, and sonication, or a combination thereof. In one aspect, the preparation of a fecal microbiota preparation involves no treatment selected from the group consisting of ethanol treatment, detergent treatment, heat treatment, irradiation, and sonication. In one aspect, the preparation of a fecal microbiota preparation involves a separation step selected from the group consisting of filtering, sieving, density gradients, filtration, chromatography, and a combination thereof. In one aspect, the preparation of a fecal microbiota preparation does not require one or more separation steps selected from the group consisting of filtering, sieving, density gradients, filtration, and chromatography. In one aspect, a fecal microbiota preparation is substantially free of non-living matter. In one aspect, a fecal microbiota preparation is substantially free of acellular material selected from the group consisting of residual fiber, DNA, viral coat material, and non-viable material. In one aspect, a fecal microbiota preparation is substantially free of eukaryotic cells from the fecal microbiota's donor.

In one aspect, the present disclosure provides a method for treating ASD in a subject in need thereof, where the method comprises administering to the subject a pharmaceutically active dose of a therapeutic composition described herein. The specific dosage and dosage range that can be used depends on a number of factors, and the determination of dosage ranges and optimal dosages for a particular patient is well within the ordinary skill of one in the art in view of this disclosure. It is further understood, however, that the specific dose level for any particular human will depend upon a variety of factors including the activity of the specific compound employed, the age, body weight, general health, gender, and diet of the human, the time of administration, the route of administration, the rate of excretion, any drug combination, and the severity of any disorder being treated.

In one aspect, the present disclosure provides a method for treating ASD in a subject in need thereof, where the method comprises administering daily to the subject a pharmaceutically active dose of a therapeutic composition described herein. In one aspect, a therapeutic composition is administered to a patient in need thereof at least once daily for at least two consecutive days. In one aspect, a therapeutic composition is administered at least once daily for at least 3, 4, 5, 6, 7, 8, 9, 10, 11, 12, 13, 14, or 15 consecutive days. In another aspect, a therapeutic composition is administered at least once daily for at least 1, 2, 3, 4, 5, 6, 7, 8, 9, 10, 11, or 12 consecutive weeks. In one aspect, a therapeutic composition is administered at least once daily for at most 4, 5, 6, 7, 8, 9, 10, 11, 12, 13, 14, 15, 16, 17, 18, 19, or 20 consecutive days or weeks. In another aspect, a therapeutic composition is administered at least once daily for at most 1, 2, 3, 4, 5, 6, 7, 8, 9, 10, 11, or 12 consecutive weeks or months. In a further aspect, a therapeutic composition is administered at least once for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to a patient in need thereof at least twice daily for at least two consecutive days. In one aspect, a therapeutic composition is administered at least twice daily for at least 3, 4, 5, 6, 7, 8, 9, 10, 11, 12, 13, 14, or 15 consecutive days. In another aspect, a therapeutic composition is administered at least twice daily for at least 1, 2, 3, 4, 5, 6, 7, 8, 9, 10, 11, or 12 consecutive weeks. In one aspect, a therapeutic composition is administered at least twice daily for at most 4, 5, 6, 7, 8, 9, 10, 11, 12, 13, 14, 15, 16, 17, 18, 19, or 20 consecutive days or week. In another aspect, a therapeutic composition is administered at least twice daily for at most 1, 2, 3, 4, 5, 6, 7, 8, 9, 10, 11, or 12 consecutive weeks or months. In a further aspect, a therapeutic composition is administered at least twice for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to a patient in need thereof at least three times daily for at least two consecutive days. In one aspect, a therapeutic composition is administered at least three times daily for at least 3, 4, 5, 6, 7, 8, 9, 10, 11, 12, 13, 14, or 15 consecutive days. In another aspect, a therapeutic composition is administered at least three times daily for at least 1, 2, 3, 4, 5, 6, 7, 8, 9, 10, 11, or 12 consecutive weeks. In one aspect, a therapeutic composition is administered at least three times daily for at most 4, 5, 6, 7, 8, 9, 10, 11, 12, 13, 14, 15, 16, 17, 18, 19, or 20 consecutive days or weeks. In another aspect, a therapeutic composition is administered at least three times daily for at most 1, 2, 3, 4, 5, 6, 7, 8, 9, 10, 11, or 12 consecutive weeks or months. In a further aspect, a therapeutic composition is administered at least three times for at least 1, 2, 3, 4, 5, 6, 7, 8, 9, 10, 11, or 12 consecutive months or years, chronically for a subject's entire life span, or an indefinite period of time.

In one aspect, the present disclosure provides a method for treating ASD in a subject in need thereof, where the method comprises administering orally to the subject a pharmaceutically active dose of a therapeutic composition comprising live, non-pathogenic, synthetic bacterial mixture or live, non-pathogenic, purified or extracted, fecal microbiota, where the dose is administered at a dosing schedule of at least once or twice daily for at least three consecutive days or weeks. In another aspect, a dose is administered at least once, twice, or three times daily for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In one aspect, the present disclosure provides a method for treating ASD in a subject in need thereof by administering a pharmaceutical composition described herein, where the method comprises a first dosing schedule followed by a second dosing schedule. In one aspect, a first dosing schedule comprises a treatment or induction dose. In one aspect, a first dosing schedule comprises a continuous dosing schedule. In another aspect, a second dosing schedule comprises a maintenance dose lower than or equal to a pharmaceutically active dose of a first dosing schedule. In another aspect, a second dosing schedule lasts for at least about 2, 4, 6, 8, 10, 12, 18, 24, 36, 48, 72, or 96 months. In one aspect, a second dosing schedule lasts permanently, for a treated subject's entire life span, or an indefinite period of time. In one aspect, a second dosing schedule is a continuous dosing schedule. In another aspect, a second dosing schedule is an intermittent dosing schedule. In a further aspect, a second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days.

In another aspect, a second dosing schedule comprises administering a second dose (e.g., a maintenance dose) every other day, every two days, or every 3, 4, 5, 6, 7, 8 days. In another aspect, a maintenance dose is administered for an extended period of time with or without titration (or otherwise changing the dosage or dosing schedule). In one aspect, the interval between a first and a second dosing schedule is at least about 1, 2, 3, 4, 5, 6, 7, 8, 9, 10, 11, or 12 weeks. In another aspect, a second dosing schedule (e.g., a maintenance dose) comprises a dosage about 2, 5, 10, 50, 100, 200, 400, 800, 1000, 5000 or more folds lower than the dosage used in a first dosing schedule (e.g., an initial treatment dose). In another aspect, a second dosing schedule (e.g., a maintenance dosing schedule) has an equal or lower dosing frequency than a first dosing schedule (e.g., an initial treatment dosing schedule). In another aspect, a second dosing schedule (e.g., a maintenance dosing schedule) has a higher dosing interval than a first dosing schedule (e.g., an initial treatment dosing schedule).

In one aspect, a first or second dosing schedule used in a method can be once-a-week, twice-a-week, or thrice-a-week. The term “once-a-week” means that a dose is administered once in a week, preferably on the same day of each week. “Twice-a-week” means that a dose is administered two times in a week, preferably on the same two days of each weekly period. “Thrice-a-week” means that a dose is administered three times in a week, preferably on the same three days of each weekly period.

In one aspect, the present disclosure provides a method for treating ASD in a subject in need thereof by administering, at a first dosing schedule, a pharmaceutical composition described herein comprising fecal microbiota from a single donor followed by a second dosing schedule comprising administering a pharmaceutical composition described herein comprising fecal microbiota from a single donor. In one aspect, a pharmaceutical composition administered at a first dosing schedule and a second dosing schedule comprises fecal microbiota derived from the same single donor. In another aspect, the pharmaceutical composition administered at a first dosing schedule comprises fecal microbiota derived from a donor different from the single donor at the second dosing schedule. In another aspect, the microbiota from a single donor comprises a substantially complete microbiota.

In one aspect, a subject being treated is a subject already with a disorder (e.g., ASD). Administration of a disclosed therapeutic composition to clinically, asymptomatic human subject who is genetically predisposed or prone to a disorder (e.g., ASD) is also useful in preventing the onset of clinical symptoms. A human subject genetically predisposed or prone to ASD can be a human subject having a close family member or relative exhibiting or having suffered a disorder (e.g., ASD). In another aspect, a subject being treated is a subject in which ASD is to be prevented. In another aspect, a subject being treated is predisposed or susceptible to a disorder (e.g., ASD). In another aspect, a subject being treated is a subject diagnosed as having a disorder (e.g., ASD). In one aspect, a subject being treated is a patient in need thereof.

In one aspect, a subject being treated is a human patient. In one aspect, a patient is a male patient. In one aspect, a patient is a female patient. In one aspect, a patient is a premature newborn. In one aspect, a patient is a term newborn. In one aspect, a patient is a neonate. In one aspect, a patient is an infant. In one aspect, a patient is a toddler. In one aspect, a patient is a young child. In one aspect, a patient is a child. In one aspect, a patient is an adolescent. In one aspect, a patient is a pediatric patient. In one aspect, a patient is a geriatric patient. In one aspect, a human patient is a child patient below about 18, 15, 12, 10, 8, 6, 4, 3, 2, or 1 year old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a patient is a young old patient (65-74 years). In one aspect, a patient is a middle old patient (75-84 years). In one aspect, a patient is an old patient (>85 years).

In one aspect, a method comprises administering a therapeutic composition orally, by enema, or via rectal suppository. In one aspect, a pharmaceutical composition is formulated as a geltab, pill, microcapsule, capsule, tablet, or a powder. In one aspect, a therapeutic composition is formulated as an enteric coated capsule or microcapsule, acid-resistant capsule or microcapsule, or formulated as part of or administered together with a food, a food additive, a dairy-based product, a soy-based product or a derivative thereof, a jelly, or a yogurt. In one aspect, a therapeutic composition is formulated as a powder, a powder for reconstitution with an appropriate diluent for naso-enteric infusion or colonoscopic infusion, a powder for reconstitution with appropriate diluent, flavoring and gastric acid suppression agent for oral ingestion, or a powder for reconstitution with food or drink. In another aspect, a therapeutic composition is formulated as an acid-resistant enteric coated capsule. A therapeutic composition can be provided as a powder for sale in combination with a food or drink. A food or drink can be a dairy-based product or a soy-based product. In another aspect, a food or food supplement contains enteric-coated and/or acid-resistant microcapsules containing a therapeutic composition.

In an aspect, a therapeutic composition comprises a liquid culture. In another aspect, a therapeutic composition is lyophilized, pulverized and powdered. It may then be infused, dissolved such as in saline, as an enema. Alternatively the powder may be encapsulated as enteric-coated and/or acid-resistant capsules for oral administration. These capsules may take the form of enteric-coated and/or acid-resistant microcapsules. A powder can preferably be provided in a palatable form for reconstitution for drinking or for reconstitution as a food additive. In a further aspect, a food is yogurt. In one aspect, a powder may be reconstituted to be infused via naso-duodenal infusion.

In another aspect, a therapeutic composition is in a liquid, frozen, freeze-dried, spray-dried, lyophilized, or powder formulation. In a further aspect, a therapeutic composition is formulated as a delayed or gradual enteric release form. In another aspect, a therapeutic composition comprises an excipient, a saline, a buffer, a buffering agent, or a fluid-glucose-cellobiose agar (RGCA) media.

In one aspect, a therapeutic composition further comprises an acid suppressant, an antacid, an H2 antagonist, a proton pump inhibitor or a combination thereof. In one aspect, a therapeutic composition substantially free of non-living matter. In another aspect, a therapeutic composition substantially free of acellular material selected from the group consisting of residual fiber, DNA, viral coat material, and non-viable material.

In one aspect, a therapeutic composition comprises a cryoprotectant. In another aspect, a cryoprotectant comprises, consisting essentially or, or consisting of polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

In another aspect, a therapeutic composition comprises a lyoprotectant. In one aspect, the same substance or the same substance combination is used as both a cryoprotectant and a lyoprotectant. Exemplary lyoprotectants include sugars such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics; and combinations thereof. In one aspect, a lyoprotectant is a non-reducing sugar, such as trehalose or sucrose. In one aspect, a cryoprotectant or a lyoprotectant consisting essentially of, or consisting of, one or more substances mentioned in this paragraph and the paragraph above.

In one aspect, a lyophilized formulation comprises trehalose. In one aspect, a lyophilized formulation comprises 2% to 30%, 3% to 25%, 4% to 20%, 5% to 15%, 6% to 10%, 2% to 30%, 2% to 25%, 2% to 20%, 2% to 15%, or 2% to 10% trehalose. In one aspect, a lyophilized formulation comprises at least 2%, 3%, 4%, 5%, 6%, 7%, 8%, 9%, 10%, or 15% trehalose. In one aspect, a lyophilized formulation comprises at most 2%, 3%, 4%, 5%, 6%, 7%, 8%, 9%, 10%, or 15% trehalose. In one aspect, a lyophilized formulation comprises about 5% trehalose.

In one aspect, a lyophilized formulation comprises trehalose and sucrose. In one aspect, a lyophilized formulation comprises between about 8% to 12% trehalose with between about 1.5% to 3.5% sucrose and between about 0.5% to 1.5% NaCl.

In one aspect, a therapeutic composition also comprises or is supplemented with a prebiotic nutrient selected from the group consisting of polyols, fructooligosaccharides (FOSs), oligofructoses, inulins, galactooligosaccharides (GOSs), xylooligosaccharides (XOSs), polydextroses, monosaccharides, tagatose, and/or mannooligosaccharides.

In an embodiment, a method for treating or preventing ASD in a subject comprises administration to the subject of: (i) a fecal microbiota (e.g., a substantially complete fecal microbiota); and (ii) one or more prebiotics. In an embodiment, a fecal microbiota (e.g., substantially complete fecal microbiota) described herein can be administered to a subject together with a prebiotic that provides a nutrient that when utilized (e.g., metabolized) by an intestinal microbiota of the subject facilitates a therapeutic effect in the subject (e.g., reduction of one or more symptoms of ASD). In another embodiment, the prebiotic can be administered to a subject at the same time as a fecal microbiota, before administration of the fecal microbiota, or after administration of the fecal microbiota. In a further embodiment, a pharmaceutical composition comprises the prebiotic and the fecal microbiota. In another embodiment, the prebiotic and the fecal microbiota are in separate therapeutic compositions. In some embodiments, the prebiotic is selected from the group consisting of an amino acid, lactic acid, ammonium nitrate, amylose, barley mulch, biotin, carbonate, cellulose, chitin, choline, fructooligosaccharides (FOSs), fructose, galactooligosaccharides (GOSs), glucose, glycerol, heteropolysaccharide, histidine, homopolysaccharide, hydroxyapatite, inulin, isomaltulose, lactose, lactulose, maltodextrins, maltose, mannooligosaccharides, nitrogen, oligodextrose, oligofructoses, oligofructose-enriched inulin, an oligosaccharide, pectin, phosphate salts, phosphorus, a polydextrose, a polyol, potash, potassium, sodium nitrate, starch, sucrose, sulfur, sun fiber, tagatose, thiamine, trans-galactooligosaccharides, trehalose, a vitamin, a water-soluble carbohydrate, a xylooligosaccharide (XOS), and a combination thereof.

In an embodiment, a method for treating or preventing ASD in a subject comprises administration to the subject of: (i) a fecal microbiota; (ii) one or more prebiotics; and (iii) one or more antibiotics. The different components of (i)-(iii) can be administered to the subject in any order. For example, a subject can be administered one or more antibiotics, followed by one or more prebiotics, followed by a fecal microbiota. In another example, the prebiotic can be administered after the fecal microbiota. For each of the above examples, it is further understood that any given component in a method of treatment can be administered multiple times. For example, an antibiotic can be administered to a subject, followed by a fecal microbiota, followed by a prebiotic, followed by a second administration of the fecal microbiota.

Prior to administration of a composition of the present disclosure, any suitable antibiotic can be administered to the subject. In some cases, the antibiotic is administered in multiple doses before a bowel cleanse is performed. In some cases, administration of the antibiotic is initiated at least seven days (e.g., at least 7, 9, 10, 12, 14, 18, or 21 days) before the bowel cleanse. In preferred aspects, the bowel cleanse is preceded by fasting of the human subject.

Following administration of an antibiotic, the subject may undergo a bowel cleanse. In exemplary aspects, the bowel cleanse comprises administering to the subject a product such as MoviPrep®, a commercial bowel prep for colonoscopy. Preferably, the bowel cleanse removes residual vancomycin and cleanses the lower gastrointestinal tract.

In exemplary aspects, the antibiotic is a non-absorbed or minimally-absorbed antibiotic such as, for example, vancomycin or rifaximin. In one aspect, a method further comprises pretreating a subject with an antibiotic composition prior to administering a therapeutic bacterial or microbiota composition. In one aspect, an antibiotic composition comprises an antibiotic selected from the group consisting of rifabutin, clarithromycin, clofazimine, vancomycin, rifampicin, nitroimidazole, chloramphenicol, and a combination thereof. In another aspect, an antibiotic composition comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof. In a further aspect, a method further comprises pretreating a subject with an anti-inflammatory drug prior to administration of a therapeutic bacterial or microbiota composition.

In exemplary aspects, the method further comprises administering to the subject a stomach acid suppressant. Stomach acid suppressants, also known as gastric acid suppressants, suitable for use according to a method provided herein include, without limitation, proton pump inhibitors (PPIs) and histamine blockers. In some cases, the stomach acid suppressant is Prilosec and is administered to the subject one or more days in advance of oral administration of purified fecal microbiota. In some cases, the stomach acid suppressant is administered one week prior to oral administration of purified fecal microbiota.

In one aspect, every about 200 mg of a pharmaceutical composition comprises a pharmacologically active dose. In one aspect, every about 75, 100, 125, 150, 175, 200, 250, 300, 350, 400, 450, 500, 750, 1000, 1500, or 2000 mg of a pharmaceutical composition comprises a pharmacologically active dose.

In one aspect, a pharmaceutically active or therapeutic effective dose comprises at least about 10⁵, 10⁶, 10⁷, 10⁸, 10⁹, 10¹⁰, 10¹¹, 10¹², or 10¹³ cfu. In another aspect, a pharmaceutically active therapeutic effective dose comprises at most about 10⁵, 10⁶, 10⁷, 10⁸, 10⁹, 10¹⁰, 10¹¹, 10¹², or 10¹³ cfu. In a further aspect, a pharmacologically active therapeutic effective dose is selected from the group consisting of from 10⁸ cfu to 10¹⁴ cfu, from 10⁹ cfu to 10¹³ cfu, from 10¹⁰ cfu to 10¹² cfu, from 10⁹ cfu to 10¹⁴ cfu, from 10⁹ cfu to 10¹² cfu, from 10⁹ cfu to 10¹¹ cfu, from 10⁹ cfu to 10¹⁰ cfu, from 10¹⁰ cfu to 10¹⁴ cfu, from 10¹⁰ cfu to 10¹³ cfu, from 10¹¹ cfu to 10¹⁴ cfu, from 10¹¹ cfu to 10¹³ cfu, from 10¹² cfu to 10¹⁴ cfu, and from 10¹³ cfu to 10¹⁴ cfu. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutic effective dose comprises at least about 10⁵, 10⁶, 10⁷, 10⁸, 10⁹, 10¹⁰, 10¹¹, 10¹², or 10¹³ cells or spores. In another aspect, a pharmaceutically active or therapeutic effective dose comprises at most about 10⁵, 10⁶, 10⁷, 10⁸, 10⁹, 10¹⁰, 10¹¹, 10¹², or 10¹³ total cells or spores. In a further aspect, a pharmacologically active or therapeutic effective dose is selected from the group consisting of from 10⁸ to 10¹⁴, from 10⁹ to 10¹³, from 10¹⁰ to 10¹², from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores. In an aspect, the pharmaceutically active or therapeutic effective dose cell count is directed to live cells. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter.

In one aspect, a therapeutic composition described and used here comprises one or more, two or more, three or more, four or more, or five or more isolated, purified, or cultured microorganisms selected from the group consisting of Prevotella, Bifidobacterium, Desulfovibrio Clostridium, Bacillus, Collinsella, Bacteroides, Eubacterium, Fusobacterium, Propionibacterium, Lactobacillus, Ruminococcus, Escherichia coli, Gemmiger, Desulfomonas, Peptostreptococcus, Bifidobacterium, Coprococcus, Dorea, and Monilia. In one aspect, a therapeutic composition described and used here comprises at least one isolated, purified, or cultured microorganisms selected from the group consisting of Prevotella, Bifidobacterium, and Desulfovibrio. In one aspect, a therapeutic composition described and used here comprises isolated, purified, or cultured microorganisms of the genus Prevotella, Bifidobacterium, and Desulfovibrio. In one aspect, a therapeutic composition described and used here comprises one or more, two or more, or three or more isolated, purified, or cultured microorganisms selected from the group consisting of Prevotella dentalis, Prevotella enoeca, Prevotella oris, Prevotella meloninogenica, Bifidobacterium bifidum, Bifidobacterium angulatum, and Desulfovibrio piger.

In one aspect, a fecal microbiota preparation described herein comprises a purified or reconstituted fecal bacterial mixture. In one aspect, a fecal microbiota preparation described and used here comprises one or more, one or more, two or more, three or more, four or more, or five or more live fecal microorganisms are selected from the group consisting of Prevotella, Desulfovibrio, Acidaminococcus, Akkermansia, Alistipes, Anaerotruncus, Bacteroides, Bifidobacterium, Blautia, Butyrivibrio, Clostridium, Collinsella, Coprococcus, Corynebacterium, Dorea, Enterococcus, Escherichia, Eubacterium, Faecalibacterium, Haemophilus, Holdemania, Lactobacillus, Moraxella, Parabacteroides, Propionibacterium, Raoultella, Roseburia, Ruminococcus, Selenomonas, Staphylococcus, Streptococcus, Subdoligranulum, and Veillonella. In one aspect, a fecal microbiota preparation comprises one or more, one or more, two or more, three or more, four or more, or five or more live fecal microorganisms are selected from the group consisting of Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium, Staphylococcus epidermidis, Eubacterium limosum, Tissirella praeacuta, Fusobacterium mortiferum I, Fusobacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tenue, Eubacterium ramulus, Bacteroides clostridiiformis ssp. clostridliformis, Bacteroides coagulans, Prevotella oralis, Prevotella ruminicola, Odoribacter splanchnicus, Desuifomonas pigra, Prevotella dentalis, Prevotella enoeca, Prevotella oris, Prevotella meloninogenica, Bifidobacterium bifidum, Bifidobacterium angulatum, and Desulfovibrio piger.

In one aspect, a fecal microbiota preparation described and used here lacks or is substantially devoid of one or more, one or more, two or more, three or more, four or more, or five or more live fecal microorganisms are selected from the group consisting of Acidaminococcus, Akkermansia, Alistipes, Anaerotruncus, Bacteroides, Bifidobacterium, Blautia, Butyrivibrio, Clostridium, Collinsella, Coprococcus, Corynebacterium, Dorea, Enterococcus, Escherichia, Eubacterium, Faecalibacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella. In one aspect, a fecal microbiota preparation lacks or is substantially devoid of one or more, one or more, two or more, three or more, four or more, or five or live more fecal microorganisms are selected from the group consisting of Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium, Staphylococcus epidermidis, Eubacterium limosum, Tissirella praeacuta, Fusobacterium mortiferum I, Fusobacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tenue, Eubacterium ramulus, Bacteroides clostridiiformis ssp. clostridliformis, Bacteroides coagulans, Prevotella oralis, Prevotella ruminicola, Odoribacter splanchnicus, and Desuifomonas pigra.

In another aspect, a therapeutic composition comprises a fecal microbiota further supplemented, spiked, or enhanced with a fecal microorganism. In one aspect, a fecal microbiota is supplemented with a non-pathogenic (or with attenuated pathogenicity) bacterium of Prevotella, Bifidobacterium, Desulfovibrio, or any combinations thereof. In one aspect, a fecal microbiota is supplemented with a non-pathogenic (or with attenuated pathogenicity) bacterium of Clostridium, Collinsella, Dorea, Ruminococcus, Coprococcus, Prevotella, Bifidobacterium, Desulfovibrio, Veillonella, Bacteroides, Bacillus, or a combination thereof. In another aspect, a therapeutic composition comprises a fecal microbiota further supplemented, spiked, or enhanced with a species of Veillonellaceae, Firmicutes, Gammaproteobacteria, Bacteroidetes, or a combination thereof. In another aspect, a therapeutic composition comprises a fecal microbiota further supplemented with fecal bacterial spores. In one aspect, fecal bacterial spores are Clostridium spores, Bacillus spores, or both. In another aspect, a therapeutic composition comprises a fecal microbiota further supplemented, spiked, or enhanced with a Bacteroides species selected from the group consisting of Bacteroides coprocola, Bacteroides plebeius, Bacteroides massiliensis, Bacteroides vulgatus, Bacteroides helcogenes, Bacteroides pyogenes, Bacteroides tectus, Bacteroides unformis, Bacteroides stercoris, Bacteroides eggerthii, Bacteroides finegoldii, Bacteroides thetaiotaomicron, Bacteroides ovatus, Bacteroides acidifaciens, Bacteroides caccae, Bacteroides nordii, Bacteroides salyersiae, Bacteroides fragilis, Bacteroides intestinalis, Bacteroides coprosuis, Bacteroides distasonis, Bacteroides goldsteinii, Bacteroides merdae, Bacteroides forsythus, Bacteroides splanchnicus, Bacteroides capillosus, Bacteroides cellulosolvens, and Bacteroides ureolyticus.

In an aspect, a therapeutic composition comprises non-pathogenic spores of one or more, two or more, three or more, or four or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum. In an aspect, a therapeutic composition comprises one or more, two or more, three or more, or four or more non-pathogenic Bacteroides species selected from the group of Bacteroides coprocola, Bacteroides plebeius, Bacteroides massiliensis, Bacteroides vulgatus, Bacteroides helcogenes, Bacteroides pyogenes, Bacteroides tectus, Bacteroides unformis, Bacteroides stercoris, Bacteroides eggerthii, Bacteroides finegoldii, Bacteroides thetaiotaomicron, Bacteroides ovatus, Bacteroides acidifaciens, Bacteroides caccae, Bacteroides nordii, Bacteroides salyersiae, Bacteroides fragilis, Bacteroides intestinalis, Bacteroides coprosuis, Bacteroides distasonis, Bacteroides goldsteinii, Bacteroides merdae, Bacteroides forsythus, Bacteroides splanchnicus, Bacteroides capillosus, Bacteroides cellulosolvens, and Bacteroides ureolyticus. The foregoing Clostridium and Bacteroides can be either cultured or purified and can be used in combination in a single combination for a synergistic effect.

In an aspect, a therapeutic composition comprises purified, isolated, or cultured viable non-pathogenic Clostridium and a plurality of purified, isolated, or cultured viable non-pathogenic microorganisms from one or more genera selected from the group consisting of Collinsella, Coprococcus, Dorea, Eubacterium, and Ruminococcus. In another aspect, a therapeutic composition comprises a plurality of purified, isolated, or cultured viable non-pathogenic microorganisms from one or more genera selected from the group consisting of Clostridium, Collinsella, Coprococcus, Dorea, Eubacterium, and Ruminococcus.

In an aspect, a therapeutic composition comprises two or more genera selected from the group consisting of Collinsella, Coprococcus, Dorea, Eubacterium, and Ruminococcus. In another aspect, a therapeutic composition comprises two or more genera selected from the group consisting of Coprococcus, Dorea, Eubacterium, and Ruminococcus. In a further aspect, a therapeutic composition comprises one or more, two or more, three or more, four or more, or five or more species selected from the group consisting of Coprococcus catus, Coprococcus comes, Dorea longicatena, Eubacterium eligens, Eubacterium hadrum, Eubacterium hallii, Eubacterium rectale, and Ruminococcus torques.

In an aspect, a therapeutic composition comprises a fecal microbiota from a subject selected from the group consisting of a human, a bovine, a dairy calf, a ruminant, an ovine, a caprine, or a cervine. In another aspect, a therapeutic composition can be administered to a subject selected from the group consisting of a human, a bovine, a dairy calf, a ruminant, an ovine, a caprine, or a cervine. In an aspect, a therapeutic composition is substantially or nearly odourless.

In an aspect, a therapeutic composition provided here comprises a fecal microbiota preparation comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a therapeutic composition comprises fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 31.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a therapeutic composition comprises a preparation of flora in proportional content that resembles a normal healthy human fecal flora.

In a further aspect, a therapeutic composition comprises fecal bacteria from at least 1, 2, 3, 4, 5, 6, 7, 8, 9, or 10 different families. In an aspect, a therapeutic composition provided here comprises a fecal microbiota comprising a weight ratio between fecal-derived non-living material and fecal-derived biological material of no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7% 8%, 9%, or 10%. In another aspect, a therapeutic composition provided here comprises a fecal microbiota comprising a weight ratio between fecal-derived non-living material and fecal-derived biological material of no greater than 20%, 25%, 30%, 35%, 40%, 45%, 50%, 55%, 60%, 65%, 70%, 75%, 80%, 85%, 90%, or 95%. In another aspect, a therapeutic composition provided here comprises, consists of, or consists essentially of, particles of non-living material and/or particles of biological material of a fecal sample that passes through a sieve, a column, or a similar filtering device having a sieve, exclusion, or particle filter size of 2.0 mm, 1.0 mm, 0.5 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, or 0.2 mm. “Non-living material” does not include an excipient, e.g., a pharmaceutically inactive substance, such as a cryoprotectant, added to a processed fecal material. “Biological material” refers to the living material in fecal material, and includes microbes including prokaryotic cells, such as bacteria and archaea (e.g., living prokaryotic cells and spores that can sporulate to become living prokaryotic cells), eukaryotic cells such as protozoa and fungi, and viruses. In one aspect, “biological material” refers to the living material, e.g., the microbes, eukaryotic cells, and viruses, which are present in the colon of a normal healthy human. In an aspect, a therapeutic composition provided or comprises an extract of human feces where the composition is substantially odorless. In an aspect, a therapeutic composition provided or comprises fecal material or a fecal floral preparation in a lyophilized, crude, semi-purified or purified formulation.

In an aspect, a fecal microbiota in a therapeutic composition comprises highly refined or purified fecal flora, e.g., substantially free of non-floral fecal material. In an aspect, a fecal microbiota can be further processed, e.g., to undergo microfiltration before, after, or before and after sieving. In another aspect, a highly purified fecal microbiota product is ultra-filtrated to remove large molecules but retain the therapeutic microflora, e.g., bacteria.

In another aspect, a fecal microbiota in a therapeutic composition used herein comprises or consists essentially of a substantially isolated or a purified fecal flora or entire (or substantially entire) microbiota that is (or comprises) an isolate of fecal flora that is at least about 90%, 91%, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material; or, a substantially isolated, purified, or substantially entire microbiota as described in Sadowsky et al., WO 2012/122478 A1, or as described in Borody et al., WO 2012/016287 A2. In one aspect, a fecal microbiota preparation comprises a weight ratio between fecal-derived non-living material and fecal-derived biological material of no greater than about 0.1%, 0.2%, 0.3%, 0.4%, 0.5%, 0.6%, 0.7%, 0.8%, 0.9%, 1%, 2%, 5%, 8%, 10%, 15%, 20%, 30%, 40$, or 50%.

In an aspect, a fecal microbiota in a therapeutic composition comprises a donor's substantially entire or non-selective fecal microbiota, reconstituted fecal material, or synthetic fecal material. In another aspect, the fecal microbiota in a therapeutic composition comprises no antibiotic resistant population. In another aspect, a therapeutic composition comprises a fecal microbiota and is largely free of extraneous matter (e.g., non-living matter including acellular matter such as residual fiber, DNA, RNA, viral coat material, non-viable material; and living matter such as eukaryotic cells from the fecal matter's donor).

In an aspect, a fecal microbiota in a therapeutic composition used herein is derived from disease-screened fresh homologous feces or equivalent freeze-dried and reconstituted feces. In an aspect, a fresh homologous feces does not include an antibiotic resistant population. In another aspect, a fecal microbiota in a therapeutic composition is derived from a synthetic fecal composition. In an aspect, a synthetic fecal composition comprises a preparation of viable flora which preferably in proportional content, resembles normal healthy human fecal flora which does not include antibiotic resistant populations. Suitable microorganisms may be selected from the following: Prevotella, Bifidobacterium, Desulfovibrio, Bacteroides, Eubacterium, Fusobacterium, Propionibacterium, Lactobacillus, Ruminococcus, Escherichia coli, Gemmiger, Clostridium, Desulfomonas, Peptostreptococcus, Bifidobacterium, Collinsella, Coprococcus, Dorea, and Ruminococcus.

In an aspect, a therapeutic composition is combined with other adjuvants such as antacids to dampen bacterial inactivation in the stomach. (e.g., Mylanta, Mucaine, Gastrogel). In another aspect, acid secretion in the stomach could also be pharmacologically suppressed using H2-antagonists or proton pump inhibitors. An example H2-antagonist is ranitidine. An example proton pump inhibitor is omeprazole. In one aspect, an acid suppressant is administered prior to administering, or in co-administration with, a therapeutic composition.

In another aspect, a therapeutic composition can be provided together with a pharmaceutically acceptable carrier. As used herein, a “pharmaceutically acceptable carrier” refers to a non-toxic solvent, dispersant, excipient, adjuvant, or other material which is mixed with a live bacterium in order to permit the formation of a pharmaceutical composition, e.g., a dosage form capable of administration to the patient. A pharmaceutically acceptable carrier can be liquid (e.g., saline), gel or solid form of diluents, adjuvant, excipients or an acid resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and combinations thereof. In another aspect, a therapeutic composition may contain auxiliary substances such as wetting or emulsifying agents, stabilizing or pH buffering agents. In an aspect, a therapeutic composition contains about 1%-5%, 5%-10%, 10%-15%, 15-20%, 20%-25%, 25-30%, 30-35%, 40-45%, 50%-55%, 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of active ingredient. In an aspect, a therapeutic composition contains about 2%-70%, 5%-60%, 10%-50%, 15%-40%, 20%-30%, 25%-60%, 30%-60%, or 35%-60% of active ingredient.

In an aspect, a therapeutic composition can be incorporated into tablets, drenches, boluses, capsules or premixes. Formulation of these active ingredients into such dosage forms can be accomplished by means of methods well known in the pharmaceutical formulation arts. See, e.g., U.S. Pat. No. 4,394,377. Filling gelatin capsules with any desired form of the active ingredients readily produces capsules. If desired, these materials can be diluted with an inert powdered diluent, such as sugar, starch, powdered milk, purified crystalline cellulose, or the like to increase the volume for convenience of filling capsules.

In an aspect, conventional formulation processes can be used to prepare tablets containing a therapeutic composition. In addition to the active ingredients, tablets may contain a base, a disintegrator, an absorbent, a binder, and a lubricant. Typical bases include lactose, sugar, sodium chloride, starch and mannitol. Starch is also a good disintegrator as is alginic acid. Surface-active agents such as sodium lauryl sulfate and dioctyl sodium sulphosuccinate are also sometimes used. Commonly used absorbents include starch and lactose. Magnesium carbonate is also useful for oily substances. As a binder there can be used, for example, gelatin, gums, starch, dextrin, polyvinyl pyrrolidone and various cellulose derivatives. Among the commonly used lubricants are magnesium stearate, talc, paraffin wax, various metallic soaps, and polyethylene glycol.

In an aspect, for preparing solid compositions such as tablets, an active ingredient is mixed with a pharmaceutical carrier, e.g., conventional tableting ingredients such as corn starch, lactose, sucrose, sorbitol, talc, stearic acid, magnesium stearate, dicalcium phosphate or gums, or other pharmaceutical diluents, e.g. water, to form a solid preformulation composition containing a homogeneous mixture of a composition of the present disclosure. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing a desired amount of an active ingredient (e.g., at least about 10⁵, 10⁶, 10⁷, 10⁸, 10⁹, 10¹⁰, 10¹¹, 10¹², or 10¹³ cfu). A therapeutic composition used herein can be flavored.

In an aspect, a therapeutic composition can be a tablet or a pill. In one aspect, a tablet or a pill can be coated or otherwise compounded to provide a dosage form affording the advantage of prolonged action. For example, a tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

In an aspect, a therapeutic composition is formulated as a delayed or gradual enteric release form. In an aspect, a delayed or gradual enteric release formulation comprises the use of cellulose acetate, polyethylene glycerol, or both. In an aspect, a delayed or gradual enteric release formulation comprises the use of a hydroxypropylmethylcellulose (HPMC), a microcrystalline cellulose (MCC), magnesium stearate, or a combination thereof. In an aspect, a delayed or gradual enteric release formulation comprises the use of a poly(meth)acrylate, a methacrylic acid copolymer B, a methyl methacrylate, a methacrylic acid ester, a polyvinylpyrrolidone (PVP), a PVP-K90, or a combination thereof. In an aspect, a delayed or gradual enteric release formulation comprises the use of a solid inner layer sandwiched between two outer layers; wherein the solid inner layer comprises the pharmaceutical composition and another component selected from the group consisting of a disintegrant, an exploding agent, an effervescent or any combination thereof, wherein the outer layer comprises a substantially water soluble, a crystalline polymer, or both. In an aspect, a delayed or gradual enteric release formulation comprises the use of a non-swellable diffusion matrix.

In another aspect, a delayed or gradual enteric release formulation comprises the use of a bilayer tablet or capsule which comprises a first layer comprising a polyalkylene oxide, a polyvinylpyrrolidone, a lubricant, or a mixture thereof, and a second osmotic push layer comprising polyethylene oxide, carboxy-methylcellulose, or both. In an aspect, a delayed or gradual enteric release formulation comprises the use of a release-retarding matrix material selected from the group consisting of an acrylic polymer, a cellulose, a wax, a fatty acid, shellac, zein, hydrogenated vegetable oil, hydrogenated castor oil, polyvinylpyrrolidine, a vinyl acetate copolymer, a vinyl alcohol copolymer, polyethylene oxide, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, a methyl cellulose, an ethylcellulose, a carboxymethylcellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, carnauba wax, a polylactic acid, polyglycolic acid, a co-polymer of lactic and glycolic acid, carboxymethyl starch, potassium methacrylate/divinylbenzene copolymer, crosslinked polyvinylpyrrolidone, polyvinyl alcohols, polyvinylalcohol copolymers, polyethylene glycols, non-crosslinked polyvinylpyrrolidone, polyvinylacetates, polyvinylacetate copolymers, or any combination thereof. In an aspect, a delayed or gradual enteric release formulation comprises the use of a microenvironment pH modifier.

In an aspect, a therapeutic composition can be a drench. In one aspect, a drench is prepared by choosing a saline-suspended form of a therapeutic composition. A water-soluble form of one ingredient can be used in conjunction with a water-insoluble form of the other by preparing a suspension of one with an aqueous solution of the other. Water-insoluble forms of either active ingredient may be prepared as a suspension or in some physiologically acceptable solvent such as polyethylene glycol. Suspensions of water-insoluble forms of either active ingredient can be prepared in oils such as peanut, corn, sesame oil or the like; in a glycol such as propylene glycol or a polyethylene glycol; or in water depending on the solubility of a particular active ingredient. Suitable physiologically acceptable adjuvants may be necessary in order to keep the active ingredients suspended. Adjuvants can include and be chosen from among the thickeners, such as carboxymethylcellulose, polyvinyl pyrrolidone, gelatin and the alginates. Surfactants generally will serve to suspend the active ingredients, particularly the fat-soluble propionate-enhancing compounds. Most useful for making suspensions in liquid nonsolvents are alkylphenol polyethylene oxide adducts, naphthalenesulfonates, alkylbenzene-sulfonates, and the polyoxyethylene sorbitan esters. In addition, many substances, which affect the hydrophilicity, density and surface tension of the liquid, can assist in making suspensions in individual cases. For example, silicone anti-foams, glycols, sorbitol, and sugars can be useful suspending agents.

In one aspect, a pharmaceutical composition is in an anaerobic package or container. In another aspect, a pharmaceutical composition further comprises an oxygen scavenger. In one aspect, a container can be made oxygen free by e.g., incorporating into the container a built in or clipped-on oxygen-scavenging mechanism, e.g., oxygen scavenging pellets as described e.g., in U.S. Pat. No. 7,541,091. In another aspect, the container itself is made of an oxygen scavenging material, e.g., oxygen scavenging iron, e.g., as described by O2BLOCK™, or equivalents, which uses a purified and modified layered clay as a performance-enhancing carrier of oxygen-scavenging iron; the active iron is dispersed directly in the polymer. In one aspect, oxygen-scavenging polymers are used to make the container itself or to coat the container, or as pellets to be added; e.g., as described in U.S. Pat. App. Pub. 20110045222, describing polymer blends having one or more unsaturated olefinic homopolymers or copolymers; one or more polyamide homopolymers or copolymers; one or more polyethylene terephthalate homopolymers or copolymers; that exhibit oxygen-scavenging activity. In one aspect, oxygen-scavenging polymers are used to make the container itself or to coat the container, or as pellets to be added; e.g., as described in U.S. Pat. App. Pub. 20110008554, describing compositions comprising a polyester, a copolyester ether and an oxidation catalyst, wherein the copolyester ether comprises a polyether segment comprising poly (tetramethylene-co-alkylene ether). In one aspect, oxygen-scavenging polymers are used to make the container itself or to coat the container, or as pellets to be added; e.g., as described in U.S. Pat. App. Pub. 201000255231, describing a dispersed iron/salt particle in a polymer matrix, and an oxygen scavenging film with oxygen scavenging particulates.

In preferred aspects, purified fecal microbiota is obtained from a carefully screened, healthy, neurotypical human donor. Microbiota is separated from fecal material collected from healthy donors, mixed with a cryopreservative, stored as a frozen liquid suspension with the cryopreservative, and thawed prior to administration in liquid form. Based on the route of administration, the purified fecal microbiota can be provided as fresh, frozen-thawed, or lyophilized live microbiota. In some cases, purified fecal microbiota is administered to a human subject in the form of an oral dose. In other cases, purified fecal microbiota is administered in the form of a rectal dose.

In another aspect, provided herein are unit dosage forms comprising purified fecal microbiota. In some cases, unit dosage forms described herein are provided as part of a kit. Such a kit could include a purified fecal microbiota dosage and, optionally, a delivery device to administer the composition to the subject or instructions for administering the dosage to a subject via an appropriate delivery route. In some cases, the dosage form comprises any suitable form of live microbiota (fresh, frozen, lyophilized, etc.) and is formulated for administration to a human subject orally, by nasogastric tube, by colonoscopy, or anally. As described herein, dosage forms suitable for kits provided herein include, without limitation, liquid solutions, capsules, tablets, powders, granules, and lyophilized forms.

In another aspect, the current disclosure also encompasses methods of monitoring the effectiveness of the MTT treatment of an autism spectrum disorder (ASD) in a subject in need thereof. In one aspect, the method comprises obtaining or having obtained a stool sample from the subject that is undergoing or has been undergone an MTT. In some aspects, the stool sample is subjected to a shotgun metagenomic sequencing to determine the relative abundance or frequency or one or more genes of interest. In some aspects the genes are functional and/or metabolic genes that including but not limited to folate biosynthesis (K04094), vitamin B-12 synthesis (K02499), oleic acid synthesis (K10254), sulfur metabolism (dissimilatory sulfate reduction) aprB (K00395), an oxidative stress protection gene (K05919, K07304), ureH, ureD (K03190), urea transporter utp (K08717), glutamate metabolism FTCD (K13990), or ornithine and arginine biosynthesis argE (K01438 or any combination thereof. In some aspects, the relative abundance of the gene in the sample is compared to the baseline relative abundance of the gene. In some aspects, the baseline corresponds to the relative abundance of the gene in a stool sample obtained from the subject before the start of MTT. In some aspects, the baseline corresponds to the relative abundance of the gene in a stool sample obtained from the subject during MTT. In some aspects, the baseline corresponds to the relative abundance of the gene in a stool sample of a subject not diagnosed with or not diagnosed with ASD. In some aspects, the baseline is a predetermined value that corresponds to a mean relative abundance in a population, for example a population of subject not diagnosed with ASD.

In another aspect, the current disclosure also encompasses methods of monitoring the gut microbiome of a subject who is undergoing or has undergone the MTT treatment for autism spectrum disorder (ASD). In one aspect, the method comprises obtaining or having obtained a stool sample from the subject that is undergoing or has undergone an MTT. In some aspects, the stool sample is subjected to a shotgun metagenomic sequencing to determine the relative abundance or frequency or one or more genes of interest. In some aspects, the relative abundance of the one or more genes of interest are predictive of the relative abundance of one or more microorganism in the gut microbiome. In some aspects the microorganism can be one or more of species of Prevotella, Bifidobacterium, Desulfovibrio, Bacteroides, Eubacterium, Fusobacterium, Propionibacterium, Lactobacillus, Ruminococcus, Escherichia coli, Gemmiger, Clostridium, Desulfomonas, Peptostreptococcus, Bifidobacterium, Collinsella, Coprococcus, Dorea, and Ruminococcus. In some aspects, the relative abundance of the gene in the sample is compared to the baseline relative abundance of the gene. In some aspects, the baseline corresponds to the relative abundance of the gene in a stool sample obtained from the subject before the start of MTT. In some aspects, the baseline corresponds to the relative abundance of the gene in a stool sample obtained from the subject during MTT. In some aspects, the baseline corresponds to the relative abundance of the gene in a stool sample of a subject not diagnosed with or not diagnosed with ASD. In some aspects, the baseline is a predetermined value that corresponds to a mean relative abundance in a population, for example a population of subject not diagnosed with ASD.

In some aspects the difference in relative abundance level of the genes involved in glutamate metabolism and/or sulfur metabolism is evident after about 5 weeks to about 5 years after treatment. In some aspects, the microbial gene relative abundance is close to that of the healthy donor after about 5 to about 10 weeks, or about 10 weeks to about 3 months, or about 3 months to about 6 months, or about 6 months to about 9 months, or about 9 months to about 12 months, or about 12 months to about 15 months, or about 15 months to about 18 months, or about 18 months to about 21 months, or about 21 months to about 24 months, or about 24 months to about 27 moths, or about 27 months to about 30 months, or about 30 months to about 33 months, or about 33 months to about 36 months or more.

In some aspects, the method for treating an autism spectrum disorder (ASD) as disclosed herein comprises modifying the gut microbiome in the subject by administering a fecal microbiota preparation, wherein the modifying is determined by determining a microorganism and/or a gene relative abundance in the subject's gut and comparing to a microorganism and/or gene abundance of a neurotypical control subject. In some exemplary aspects, the method comprises obtaining either a single stool sample or multiple stool samples over a period from a subject after about 10 weeks-5 years of administration, sequencing the stool samples; wherein the sequencing comprises a shotgun metagenomic sequencing; determining a relative abundance level of at least one microorganism or at least one gene or both, comparing the relative abundance level of the at least one microorganism or the at least one gene with an abundance baseline of a neurotypical control subject. In some aspects, the method can be used to suggest further treatment options.

In some aspects, the current disclosure also encompasses a kit comprising a container for obtaining stool samples from a patient in need thereof and necessary reagents to conduct a shotgun metagenomic analysis on the sample. In some aspects, kit comprises a container and a label or package insert on or associated with the container. The containers may be formed from a variety of materials such as glass, plastic, paper etc. The kit may further include other materials desirable from a commercial and user standpoint, including other buffers, diluents, filters, needles, syringes, magnetic beads, enzymes, polynucleotides etc. A “package insert” is used to refer to instructions customarily included in commercial packages of products, that contain information about usage etc. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

In some aspects, the current disclosure also encompasses a computer implemented method to monitor the effectiveness of an MTT treatment. In some aspects, the computer implemented method is operable in obtaining at least one metagenomic dataset or results thereof and comparing it to one or more metagenomic datasets or results thereof, to compare the relative abundance level of the at least one gene with a baseline gene relative abundance level of the gene(s) to determine a difference between the baseline gene relative abundance level and the gene relative abundance level after the treatment period; wherein the difference between the baseline gene relative abundance and the gene relative abundance level after the treatment period is indicative of the effectiveness of the treatment.

### EXAMPLES

The following examples are included to demonstrate aspects of the disclosure. It should be appreciated by those of skill in the art that the techniques disclosed in the examples that follow represent techniques discovered by the inventor to function well in the practice of the present disclosure, and thus can be considered to constitute modes for its practice. However, those of skill in the art should, in light of the present disclosure, appreciate that many changes can be made in the specific aspects which are disclosed and still obtain a like or similar result without departing from the spirit and scope of the present disclosure.

The Examples 1-4 below were conducted using the exemplary techniques provided herein for guidance, though equivalent techniques known to those skilled in the art can be used.

### Exemplary Techniques

In exemplary methods as disclosed in Examples 1-4 below, the following techniques were performed as detailed below.

### Outline of the Trial

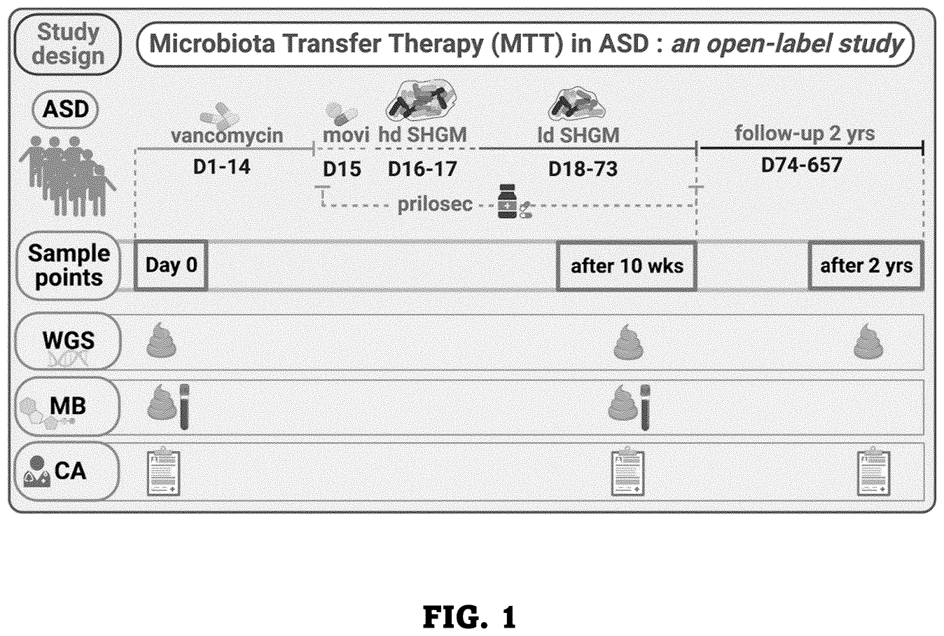

Twenty typically developing (TD) children and 18 children with Autism Spectrum Disorder (ASD) within the age range of 7-16 years were recruited. During the 10 weeks of the trial, the ASD group was treated with vancomycin for 2 weeks, given a one-day bowel cleanse with MoviPrep, then 1-2 days of high-dose liquid microbiota (Major donor) and 7-8 weeks of low-dose liquid microbiota (maintenance donor) and Prilosec (stomach acid suppressant) (FIG. 1). A follow-up evaluation was conducted at 8 weeks post-treatment (i.e., 10 weeks from Day 0), and at 2 years post-MTT (only 16 out of 18 provided fecal samples). All participants' characteristics and their medical and diet history were recorded. There was no change in diet during the MTT treatment, and after two years of treatment diet was recorded.

### Metagenomics Sequencing

Fecal DNA was extracted for sequencing. The DNA samples were from three distinct timepoints: ASD Baseline (n=18), at the end of the 10-week treatment (MTT-10 wk, n=18), and at the 2-year follow-up (MTT-2 yr, n=16). Samples were also collected from the TD (n=20), major donor (n=5) and maintenance donor (n=2) cohorts. For shotgun metagenomics, DNA was sequenced on the Illumina NextSeq 500 platform (Illumina, CA, USA) to generate 2×150 bp paired-end reads at greater sequencing depth with a minimum of 10 million reads.

### Sequencing Analysis

About 28,470,588+9,835,000 (mean±SEM) reads per sample were received from shotgun metagenomic sequencing. The quality of raw reads was examined with MultiQC. Adapters from the reads and low-quality reads with length <50 bp or phred <30 were removed. To avoid human genome contamination, all the reads were mapped against the UCSC Genome Browser's hg38 human genome reference database using a Bur-rows-Wheeler aligner (bwa) [62] and discarded mapped reads. Reads that were declared unmapped (without human genome) were used for downstream analyses.

Bacterial taxonomic composition was characterized using Kraken2 (v2.0.7, https://github.com/DerrickWood/kraken2) with the NCBI RefSeq database [63]. Kraken2 is an ultrafast taxon-assigning tool that uses the exact alignment of k-mers in a sequence and then finds the LCA (lowest common ancestor) taxa by comparing against the database. Following taxa assignment, the species-level sequence abundance estimation algorithm Bracken (Bayesian re-estimation of Abundance with KrakEN, v2.6) [64] was used to re-estimate the abundance of assigned taxa and calculate their relative abundance. Our taxonomic assignment was for bacterial phylotypes. This work uses “bacteria” or “bacterial species” terms for “phylotypes” throughout the text.

HUMAnN2 (the HMP unified metabolic analysis network, v2, https://github.com/biobakery/humann/tree/2.9) was used to identify the functional genes/pathways associated with microbiome gene markers [65]. HUMAnN2 works with MetaPhlAn2 (Metagenomic Phylogenetic Analysis, v2, https://github.com/biobakery/MetaPhlAn2) and its ChocoPhlAn pangenome database, and uses the MetCyc, MinPath and UniRef90 databases [65]. From HUMAnN2 output, gene family abundance that had a 90% match with UniRef90 were used to convert this information to KOs (KEGG Orthologs: functional genes). Subsequently, the relative abundance was calculated using all KOs' absolute abundance for each sample.

### Fecal and Plasma Metabolomics

An untargeted metabolomics approach was performed previously to measure fecal and plasma metabolites using ultra-high-performance liquid chromatography tandem mass spectroscopy (UHPLC-MS/MS). In this study, downstream analyses were further performed with shotgun metagenomics. Sample preparation and metabolites measurement was conducted as described. In brief, peak area integration and relative intensity was used to measure the metabolites and were normalized such that the median was set equal to 1. Imputation was performed for missing values by taking the lowest value of each measured metabolite divided by the square root of 2. As a threshold for missing values, the presence of metabolites was considered if it was present at a minimum of 39% in the samples. Lower than this threshold, metabolites were excluded for the analysis. Metabolomic analysis was done for ASD at baseline, at the end of MTT-10 wks, and for the TD group. While collected, measurements taken at the 2-years follow up timepoint could not be compared to prior samples due different samples batch preparation between before and after 2 years of MTT.

### GI and ASD Symptom Assessment

All GI and ASD symptom measurements are described in detail in [11]. In brief, for GI symptoms, a revised version of the Gastrointestinal Symptom Rating Scale (GSRS) with the five domains of Abdominal Pain, Reflux, Indigestion, Diarrhea, and Constipation was used. Daily stool records (DSR) using the Bristol Stool Form scale were also recorded. For ASD symptoms, Parent Global Impressions-III (PGI-III), Childhood Autism Rating Scale (CARS), Aberrant Behavior Checklist (ABC), Social Responsiveness Scale (SRS), and Vineland Adaptive Behavior Scale II (VABS-II) measurements were taken for all ASD participants.

### Diversity Index Calculation

To explore the taxonomy and KO's changes before and after MTT, alpha-diversity (Shannon index) and beta-diversity (Jaccard and Bray-Curtis dissimilarity indices) were calculated. Initially, taxonomical and KO abundance data were imported to Qiime2 (v2022.2) for calculation of the diversity indices. For the Shannon diversity index, univariate analysis was performed (Wilcoxon signed rank test for paired and Mann-Whitney tests for unpaired) and visualized using the ggpubr R package (v0.4.0). For beta-diversity, pairwise ANOSIM (analysis of similarities) with 999 permutations was used for the statistical comparisons in Qiime2 and visualized using the Dokdo API in python with Qiime2. P-values were corrected with the Benjamini-Hochberg method and assigned as q-values.

### Multi-Omics Correlation Network

To understand the interaction between bacteria, metabolites, and functional gene abundance with GI and ASD symptoms, a correlation network was derived. To make the network, relative abundances of bacteria and KOs, metabolites in Z-scores, and GI and ASD symptom severity scale data were used for the correlations. No additional data transformation was employed for the correlations. The relationships between variables were characterized as being meaningful using the Pearson's correlation coefficient (R>±0.6, p<0.05) subjected to leave-one-out FDR correction (p<0.05). FDR-corrected correlation coefficients and p-values for each variable were subjected to Cystoscope (v3.8.2) to construct and visualize the correlation network. Unconnected nodes were excluded from the network. In this study, only plasma metabolites data were used as fecal metabolites did not show significant change before and after MTT.

### Statistical Analysis and Plots

Univariate analysis comparing the sample distributions was performed for taxa and pathway data via hypothesis testing with false discovery rates determined using the leave-one-out approach. The issue of multiple hypothesis testing was addressed by determining the false discovery rate (FDR) for each significant finding (p<0.05) using a leave-one-out approach and considering p<0.05 as significant. In each group, more than 20% of samples with zeros in relative abundance (for both taxa and KOs) were filtered out during univariate analysis. Since this is a pilot study, some non-significant (p>0.05) changes are also reported that are of possible interest and indicated as “non-significantly” throughout.

When comparing unpaired data, such as between the TD and ASD baseline cohorts, both sample sets were initially tested for normality. D'Agostino and Pearson's test were used for evaluating both sample sets separately. Dependent on if the normality assumption was accepted or rejected, the samples were either compared using an F-test or 2-sample Kolmogorov-Smirnov test, respectively. The F-test was used when both sample sets were drawn from normal distributions, and this test was performed to determine if the variance was significantly different between the two groups. In the case in which the normality assumption likely held true (p-value>0.05) and there was equal variance, the 2-sample t-test was performed. In the situation when normality was observed and the variance was observed to be unequal, a Welch's test was used.

Alternatively, if one or both sample sets were evaluated and determined to be derived from a non-parametric distribution, the 2-sample Kolmogorov-Smirnov test was used to determine if the same underlying non-parametric distribution was present for both. A Mann-Whitney test was used if the two groups were observed to follow the same non-parametric distribution. In cases where different distributions were observed between samples, both sample sets were adjusted by their means and then the 2-sample Kolmogorov-Smirnov test was used again to evaluate the distribution structure. The Welch's test was used if both sample sets were still tested to be derived from different distributions, and the Mann-Whiney test was used if the distributions were determined by testing to be similar.

Taxa and pathway/KOs measurements taken post-treatment at the MTT-10 wk and MTT-2 yr time points were analyzed and compared to their baseline counterparts via optimized paired hypothesis testing as well. Normality testing via the D'Agostino and Pearson's test was used to determine whether to use a parametric or non-parametric test. When the normality assumption held for both sample sets, a paired t-test was performed. Alternatively, when either sample set was not normally distributed, the Wilcoxon matched-pairs signed rank test was used. All univariate comparisons were made for all groups against baseline.

To account for multiple hypothesis testing, the false discovery rate was determined using a leave-one-out method. This technique proceeds iteratively on each data entry, i.e., the measurements taken for the pathway or taxa data. Using the same test selection protocol outlined above for both the paired and unpaired variants, the p-value is recalculated with one individual's measurements excluded from the analysis. This is repeated such that every combination with one sample being excluded is assessed. The FDR for each variable is the ratio of the number of p-values greater than 0.05 to the total amount of p-values that were evaluated. A finding was deemed statistically significant if the FDR value determined was under 0.10.

All taxonomical and KOs plots were made with Python (v3.8.5) in JUPYTER Notebook (v6.1.4) using NUMPY (v1.19.2), MATPLOTLIB (v3.3.2), SEABORN (v0.11.0), PANDAS (v1.1.3), SCIPY (v1.5.2), etc. The area under the receiver operating characteristic (AU-ROC) was calculated in MATLAB (v2018B). For metabolic pathway images, METACYC (Metacyc.org) was used. BIORENDER and INKSCAPE (v1.1) were used to create or edit the figures.

### Example 1: Shift in Gut Bacterial Species after MTT

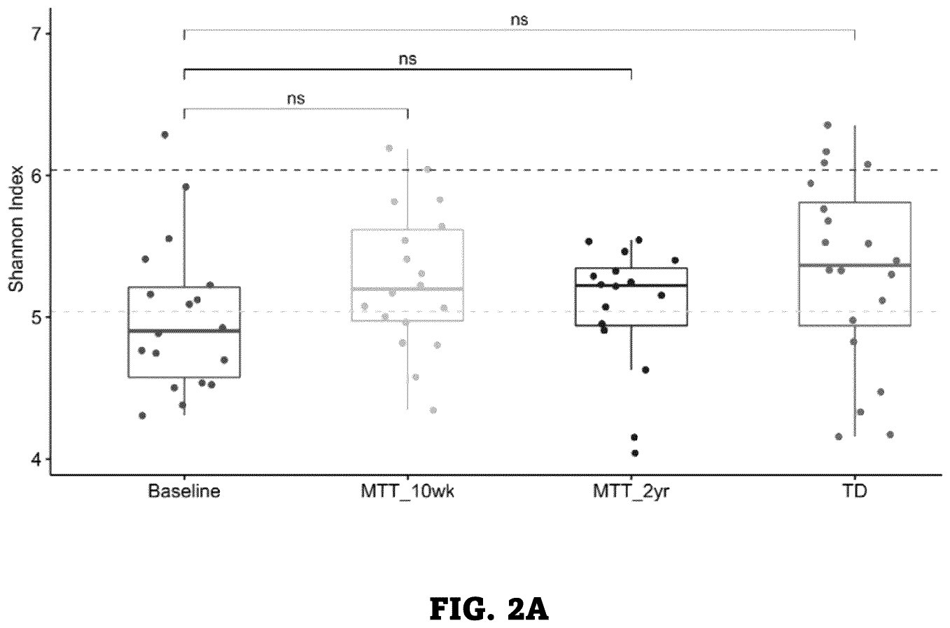

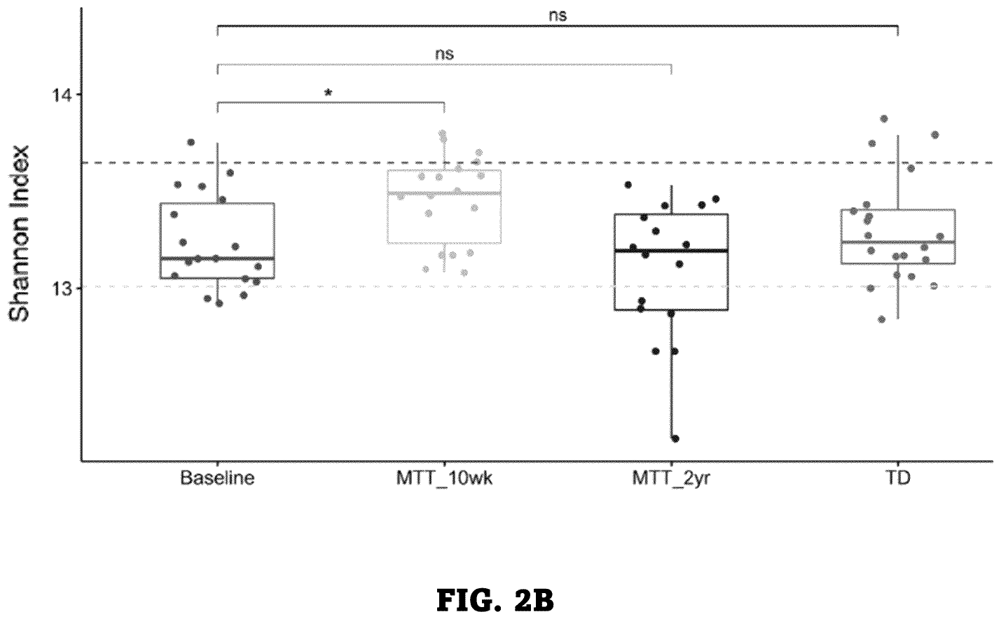

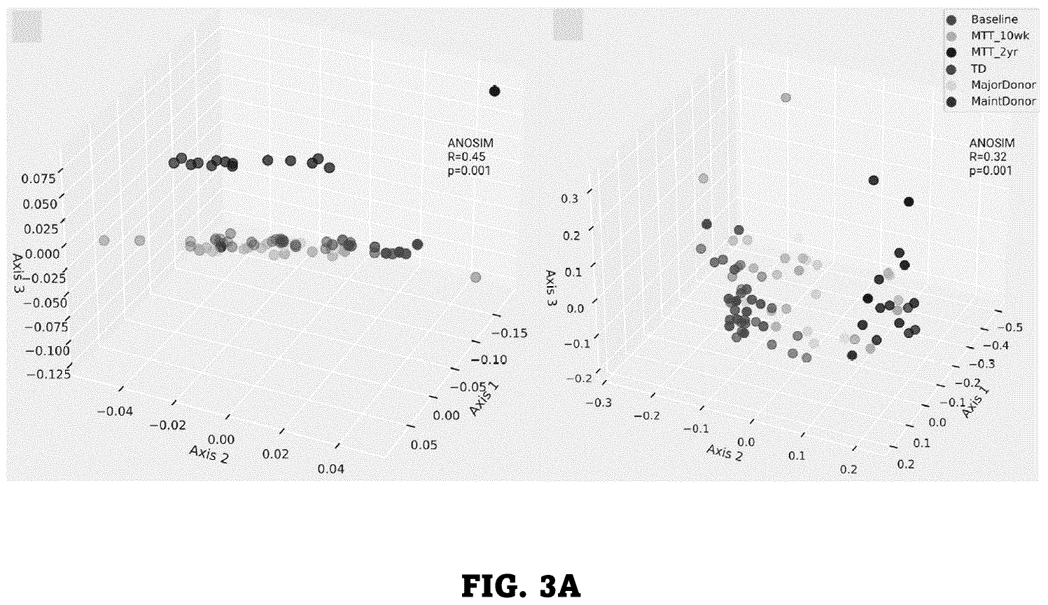

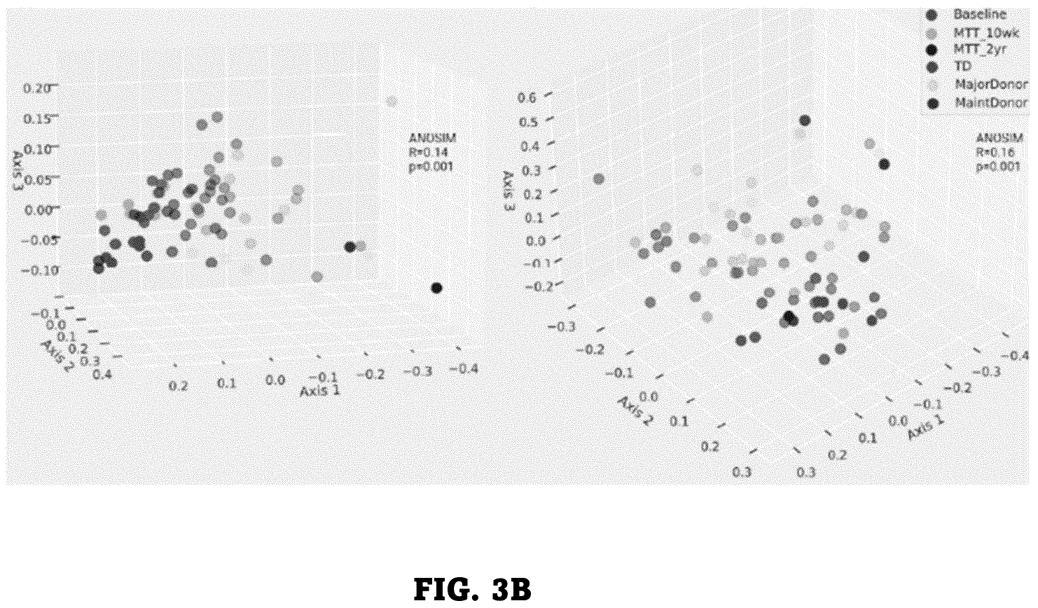

To investigate the global taxonomical changes before and after MTT (10 wk, 2 yr) in children with ASD (outline of the trial, FIG. 1), alpha and beta diversity indices were used (FIG. 2, Tables 1 and 2). This included the Shannon index (FIG. 2) and Jaccard index (FIG. 3). ANOSIM (ANalysis Of Similarities) was used for the statistical comparison between ASD Baseline and other groups. All p-values are corrected by Benjamin-Hochberg method and assigned as q-values. For the bacterial diversity, unlike Jaccard, no distinct separation was observed between all groups for Bray-Curtis distance (weighted measurement) (FIG. 3A, Table 2A), though overall distance between all groups was significant (ANOSIM R=0.14, p=0.001), suggesting dominant taxa were not significantly different between all groups. For KOs beta-diversity, no distinct separation was observed between all groups for Bray-Curtis distance (FIG. 3B, Table 2B), though overall distance between all group was significant (ANOSIM R=0.16, p=0.001). Bray-Curtis results suggested that dominant taxa and (KEGG Orthologs) KOs did not change significantly in ASD children after MTT and also not significantly different compared to TD. The Shannon index did not show any significant differences when comparing the ASD Baseline (referred to as Baseline) to after MTT or the TD (FIG. 2A), although the Baseline median for ASD was lower than all other groups. The Jaccard dissimilarity index showed a significant separation (ANOSIM R=0.45, p=0.001) only between MTT-2 yr and other groups (FIG. 2A, Table 1).

TABLE 1
Beta Diversity: Jaccard dissimilarity index

| | | | | | |
|---|---|---|---|---|---|
| Group 1 | Group 2 | Permutations | R | p-value | q-value |

A) Bacterial taxonomy

| | | | | | |
|---|---|---|---|---|---|
| Baseline | MTT_10 wk | 999 | 0.2910 | 0.001 | 0.0025 |
| | MTT_2 yr | 999 | 0.8225 | 0.001 | 0.0025 |
| | TD | 999 | 0.0539 | 0.066 | 0.108 |

B) KOs (KEGG Orthologs)

| | | | | | |
|---|---|---|---|---|---|
| Baseline | MTT_10 wk | 999 | 0.2605 | 0.001 | 0.003 |
| | MTT_2 yr | 999 | 0.3292 | 0.001 | 0.003 |
| | TD | 999 | 0.0619 | 0.027 | 0.040 |

Overall ANOSIM test values for bacterial distance R = 0.45, p = 0.001 and for KOs distance R = 0.32, p = 0.001.

TABLE 2
Beta Diversity: Bray-Curtis dissimilarity index

| | | | | | |
|---|---|---|---|---|---|
| Group 1 | Group 2 | Permutations | R | p-value | q-value |

A) Bacterial taxonomy

| | | | | | |
|---|---|---|---|---|---|
| Baseline | MTT_10 wk | 999 | 0.0342 | 0.135 | 0.16875 |
| | MTT_2 yr | 999 | 0.1259 | 0.012 | 0.03 |
| | TD | 999 | 0.0516 | 0.077 | 0.1155 |

B) KOs (KEGG Orthologs)

| | | | | | |
|---|---|---|---|---|---|
| Baseline | MTT_10 wk | 999 | 0.1275 | 0.004 | 0.0128 |
| | MTT_2 yr | 999 | 0.1555 | 0.001 | 0.0128 |
| | TD | 999 | 0.0402 | 0.112 | 0.1360 |

Overall ANOSIM test values for bacterial distance R = 0.14, p = 0.001 and for KOs distance R = 0.16, p = 0.001.

Shotgun metagenomic analysis revealed 5272 unique bacterial species in children of the ASD and TD cohorts (Tables 1-3). Comparing the sequences in the ASD cohort at Baseline vs. TD, we Identified 371 bacterial species that had significantly lower relative abundance (adjusted p<0.05) compared to the TD group, and none were significantly higher. For these 371 bacterial taxa that were initially lower in the ASD cohort, after MTT-10 wks, the relative abundance of 98 (out of 371) bacterial species significantly changed (adjusted p<0.05); 97 significantly increased and 1 decreased (Alistipes finegoldii) in the MTT-10 wks group compared to Baseline. However, at MTT-2 yrs the relative abundance of 60 (out of 371) bacterial species were significantly decreased (adjusted p<0.05) compared to Baseline, becoming less similar to the TD cohort (Tables 1-3).

TABLE 3
Taxonomy and KOs comparison between groups
A) Bacterial taxonomy comparisons (all 5,272 taxa)

| ASD at Baseline | ASD at Baseline vs. | | |
|---|---|---|---|
| vs. TD | MTT-10 wks | MTT-2 yrs | differences |
| 0 | 650 | 12 | Higher/increased |
| | | | compared to Baseline |
| 371 | 16 | 1599 | Lower/decreased |
| | | | compared to Baseline |
| 371 | 666 | 1611 | Total |

ASD at Baseline vs. (out of 371 taxa
different between Baseline ASD vs. TD)

| MTT-10 wks | MTT-2 yrs | differences |
|---|---|---|
| 97 | 0 | Increased compared |
| | | to ASD Baseline |
| 1 | 60 | Decreased compared |
| | | to ASD Baseline |
| 98 | 60 | Total |

B) KOs (KEGG Orthologs) comparisons (all 5,069 KOs)

| ASD at Baseline | ASD at Baseline vs. | | |
|---|---|---|---|
| vs. TD | MTT-10 wks | MTT-2 yrs | differences |
| 15 | 149 | 13 | Higher/increased |
| | | | compared to Baseline |
| 22 | 34 | 196 | Lower/decreased |
| | | | compared to Baseline |
| 37 | 183 | 209 | Total |

ASD at Baseline vs. (out of
Baseline vs. TD = 37 KOs)

| MTT-10 wks | MTT-2 yrs | differences |
|---|---|---|
| 9 | 3 | Increased compared |
| | | to ASD Baseline |
| 01 | 12 | Decreased compared |
| | | to ASD Baseline |
| 10 | 15 | Total |

Univariate comparison of the relative abundance (after log₁₀ transformation) of bacterial species (n = 5,272) and KEGG Orthologs (KOs, n = 5,069) between groups. All statistically significant numbers are with adjusted p < 0.05.

When analyzing all 5272 species, the longitudinal pairwise comparison between baseline and MTT-10 weeks showed 666 taxa were significantly different; abundance of 650 bacterial species increased and 16 decreased significantly (adjusted p<0.05, Table 1). After MTT-2 yrs, 1611 taxa were significantly different to baseline (adjusted <0.05); 12 increased (Tables 1 and 2) and 1599 decreased in MTT-2 yrs compared to Baseline. This suggests that MTT initially led to an increase in relative abundance of 650 species, but at 2 years only 12 had higher relative abundance. Similarly, MTT initially decreased the relative abundance of only 16 species, but at 2 years 1599 were at lower relative abundance. Comparison of TD vs. MTT data (10 wks, 2 yrs) for all bacteria showed similar trend to ASD Baseline vs. MTT (Tables 1 and 2). So, initial changes occurred after MTT-10 wks, but additional major changes had occurred at 2-years post-treatment. Although changes led initially to more similarity to the TD group, at the 2-year follow-up the ASD group had formed a microbiome that was very distinct from its initial Baseline, and was distinct from the TD group (Tables 1 and 2).

Comparing data of TD vs. MTT-10 wks, 435 bacterial species were significantly different (adjusted p<0.05); 48 were higher and 387 were lower in MTT-10 wks. For TD vs. MTT-2 yrs, 2990 bacterial species were significantly different (adjusted p<0.05); 6 were higher (Tables 2 and 3) and 2984 were lower after MTT-2 yrs compared to TD (Tables 2 and 3). The above findings suggest that bacterial abundance became less similar after MTT-2 yrs compared to TD, so after 2-years ASD children developed their own different gut microbiome compared to all other groups.

### Example 2: Change in Specific Bacterial Species after MTT Including Fiber-Consuming, Probiotic and Sulfur-Reducing Bacteria

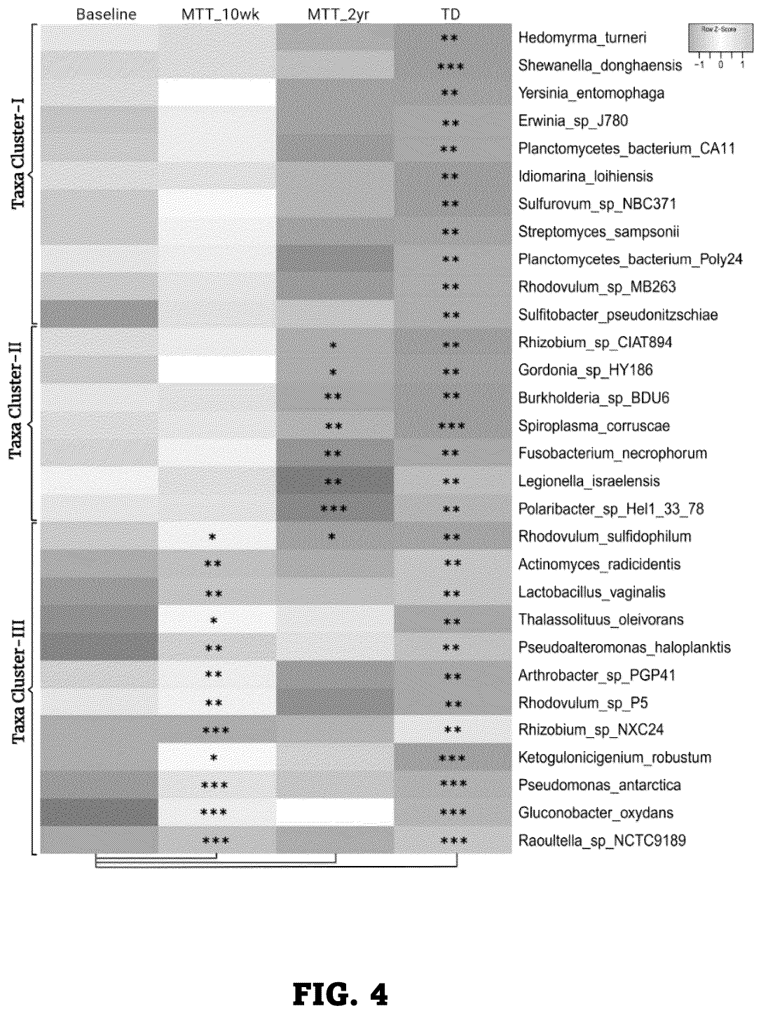

FIG. 4 illustrates the differences in the top 30 differentially abundant between ASD and TD groups (cutoff p<0.01, adjusted p<0.05) and changes after MTT compared to Baseline in children with ASD. Taxa Cluster-I shows the 11 bacteria which were significantly lower (cutoff p<0.01, adjusted p<0.05) in the Baseline group compared with the TD group. At MTT-10 wk, bacterial abundance slightly increased but no statistically significant change was observed compared to Baseline. However, at 2 years the relative abundance had generally decreased (non-significantly, p>0.05) back to relative abundance at Baseline. Note that there were no bacteria that were significantly higher at Baseline in the ASD group compared to the TD group.

Taxa Cluster-II shows the 7 bacterial taxa with significantly lower (cutoff raw p<0.01, adjusted p<0.05) in relative abundances at Baseline group compared to TD, and which did not change significantly at MTT 10 weeks, but significantly decreased (adjusted p<0.05) at MTT-2 yr compared to Baseline. Taxa Cluster-III shows the 12 bacteria whose relative abundances were significantly lower (cutoff raw p<0.01, adjusted p<0.05) in the Baseline group compared to TD and which significantly increased (adjusted p<0.05) at MTT-10 wk compared to Baseline group. At MTT-2 yr, out of 12, only 1 bacterial species (Rhodovulum sulfidophilum) significantly decreased (adjusted p<0.05) and there was no change for others (11 out of 12) compared to Baseline.

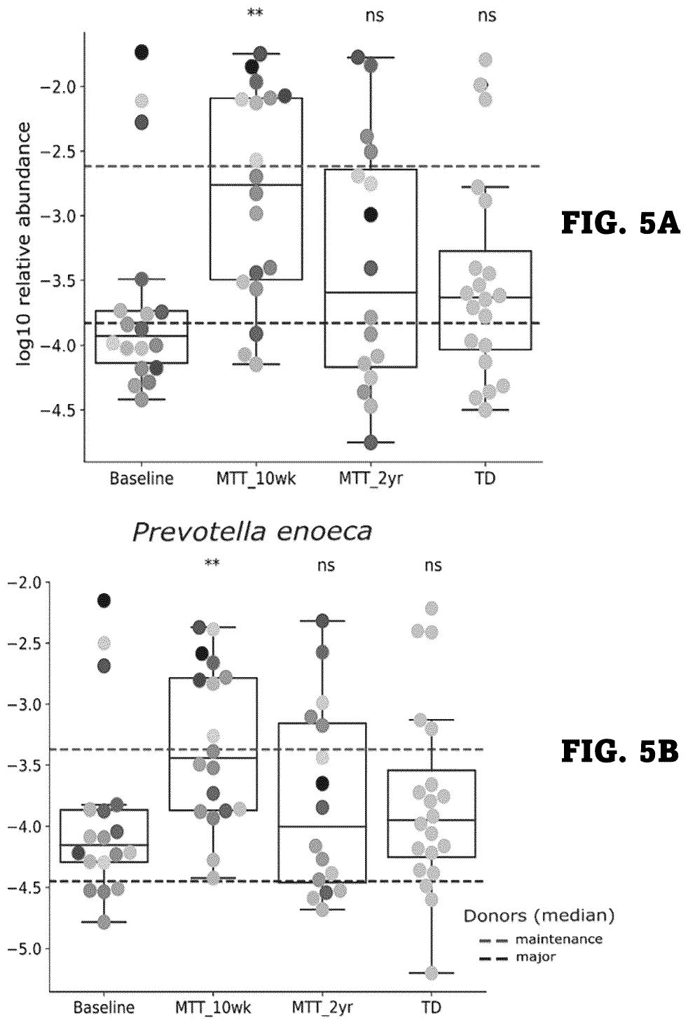

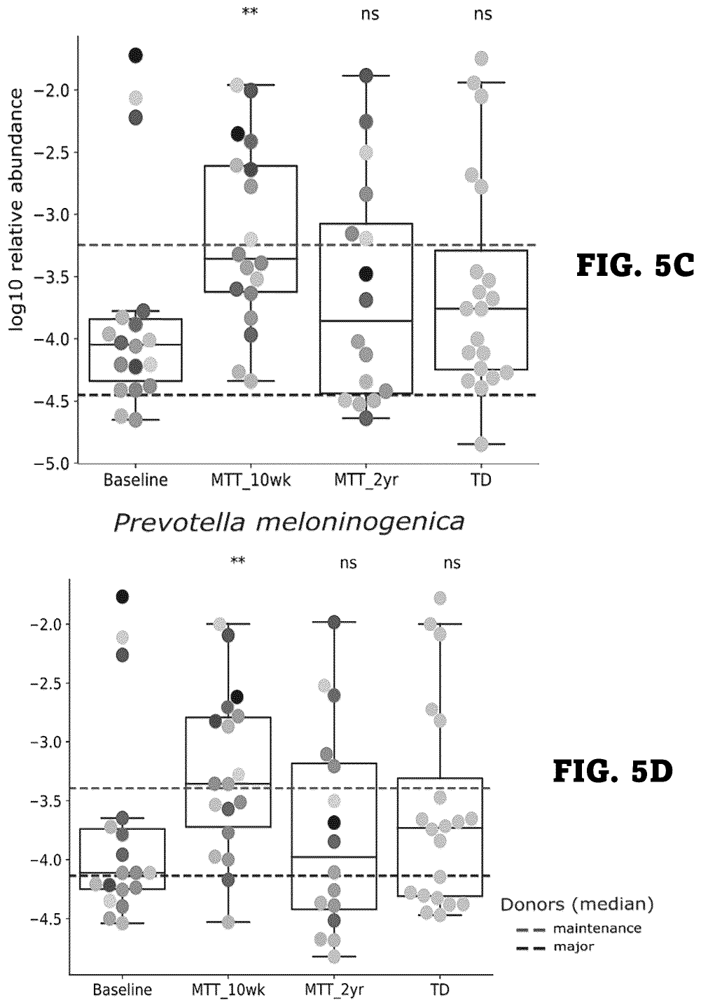

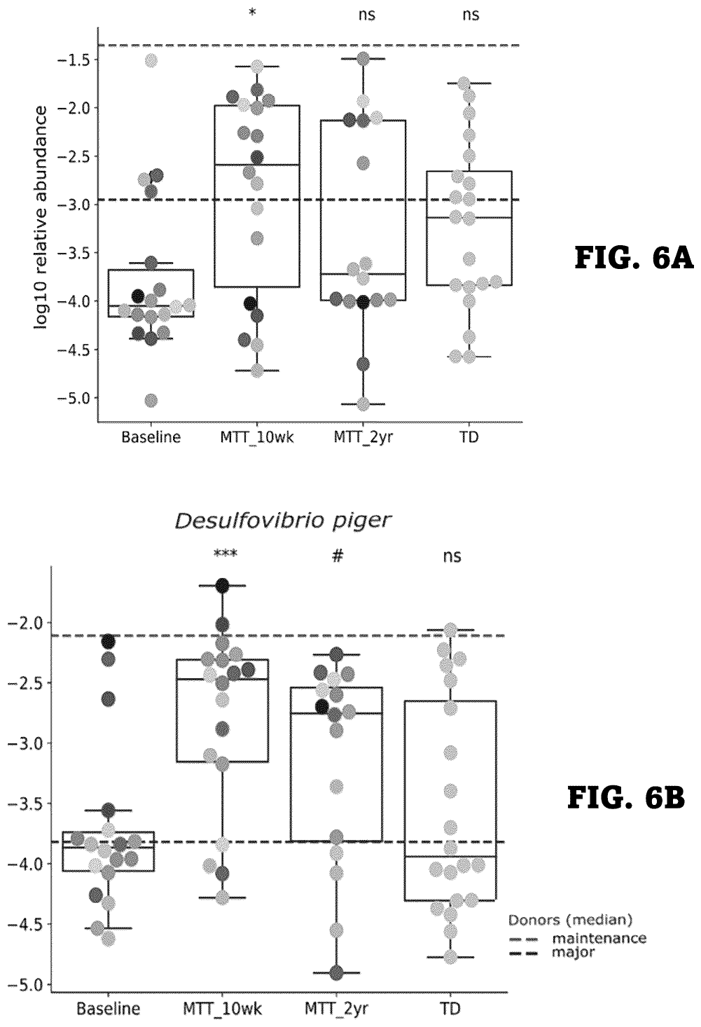

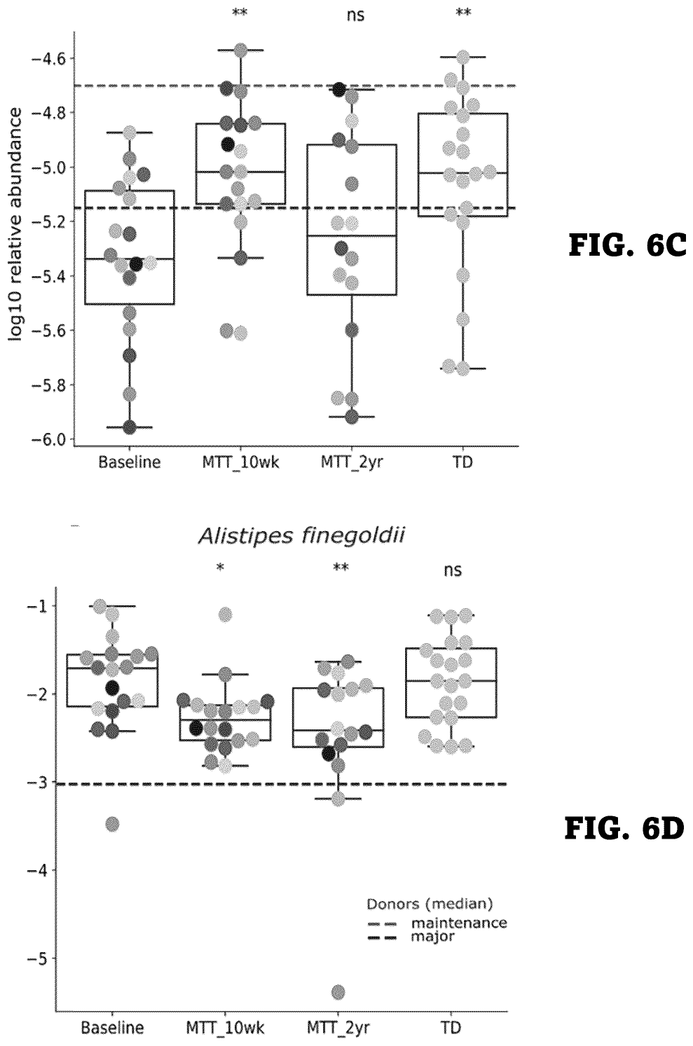

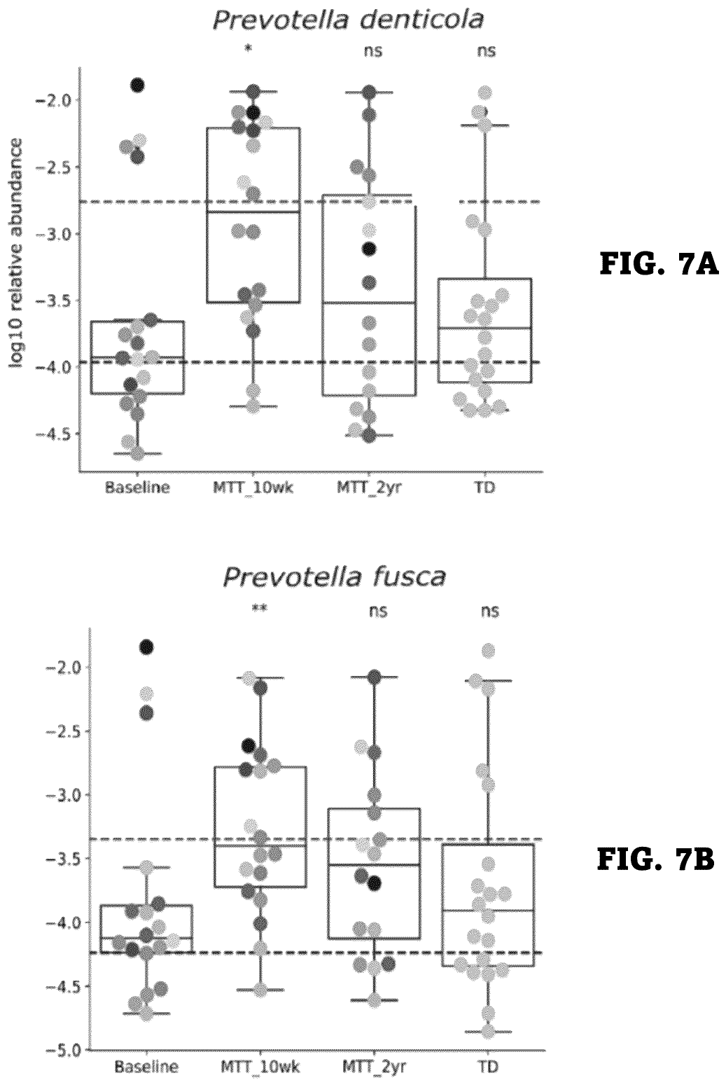

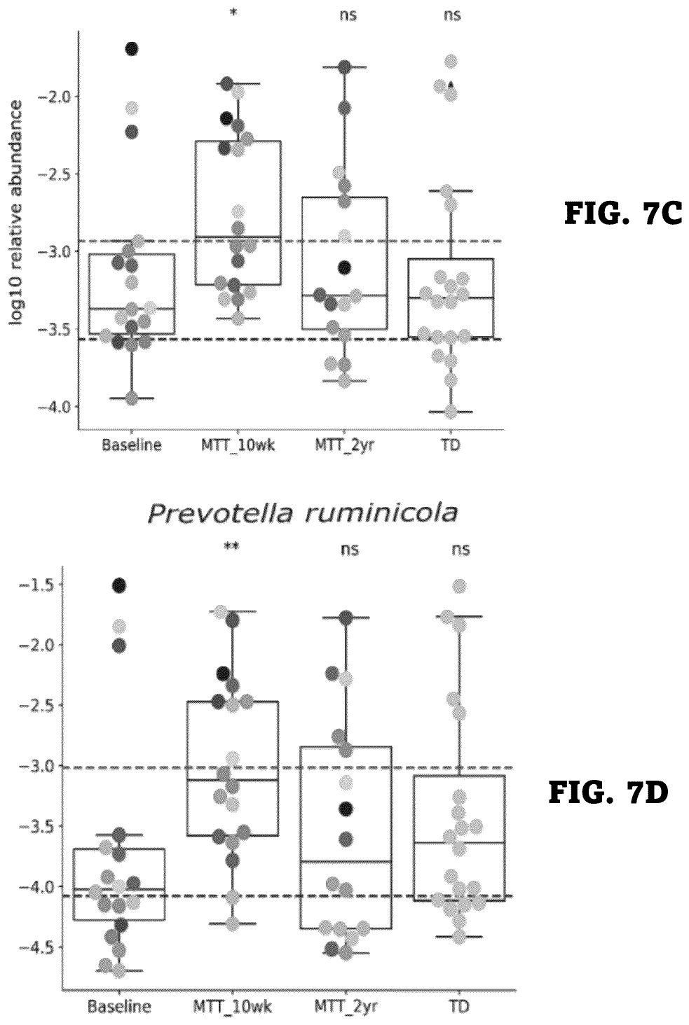

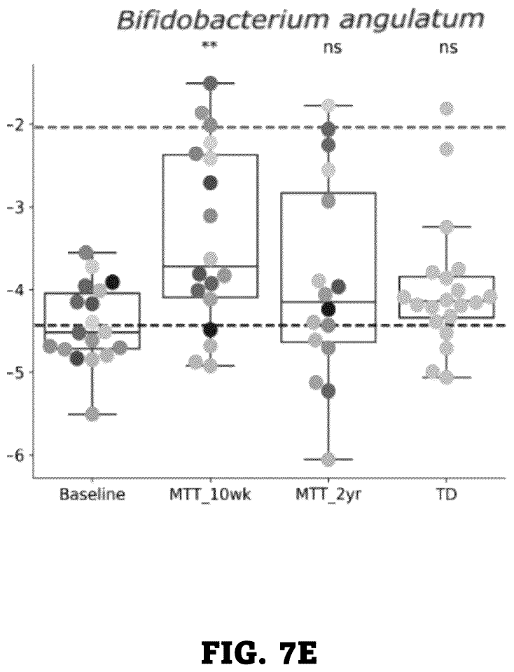

Initial targeted focus of the study was on Prevotella (fiber-consuming bacteria), Bifidobacterium (common probiotic), and Desulfovibrio (sulfur-reducing), since 16S rRNA gene amplicon study with the same samples found that these genera significantly increased after MTT. Species level changes were examined, and it was found that ASD at Baseline had lower median levels of specific species of Prevotella, Bifidobacterium and Desulfovibrio compared to TD (raw p<0.05, adjusted p>0.05) (FIGS. 5A-5D and 6A-6D), though it was not statistically significant after FDR correction. As seen in FIGS. 5 and 6, the relative abundance of several Prevotella, Bifidobacterium and Desulfovibrio species significantly increased (adjusted p<0.05) at MTT-10 wk; at MTT-2 yr, the relative abundance decreased when compared to 10 weeks but remained non-significantly higher than Baseline (median, p>0.05), except for D. piger, which remained significantly higher. Eight Prevotella species were obtained that increased significantly after MTT-10 wk, including P. denatalis, P. enoeca, P. oris, P. meloninogenica (FIG. 5A-5D); P. denticola, P. fusca, P. intermedia, and P. ruminicola (FIG. 7A-7D). For Bifidobacterium, two species increased significantly (B. bifidum, (FIG. 6A); B. angulatum, FIG. 7E), whereas for Desulfovibrio, only D. piger increased significantly and remained at higher relative abundance after 2 years (FIG. 6B).

Due to higher taxonomical variation (Table 3), the analysis was focused on a few specific bacteria (FIGS. 5 and 6) previously reported as beneficial or associated with ASD or neurological disorders. As seen in FIG. 6C, the relative abundance of lactic acid-producing bacteria Lactobacillus vaginalis was significantly lower at Baseline vs. TD (adjusted p<0.05), and its relative abundance increased (adjusted p<0.05) at MTT-10 wk compared to ASD Baseline, but decreased somewhat at MTT-2 yr. Alistipes finegoldii (FIG. 6D), which was previously found in other studies to be significantly higher in ASD and linked with ASD, was non-significantly higher (median, p>0.05) at Baseline vs. TD and significantly decreased at MTT-10 wk vs. Baseline (adjusted p<0.05), and remained significantly lower at MTT-2 yr (adjusted p<0.05) in children with ASD and closer to the TD levels (FIG. 7D).

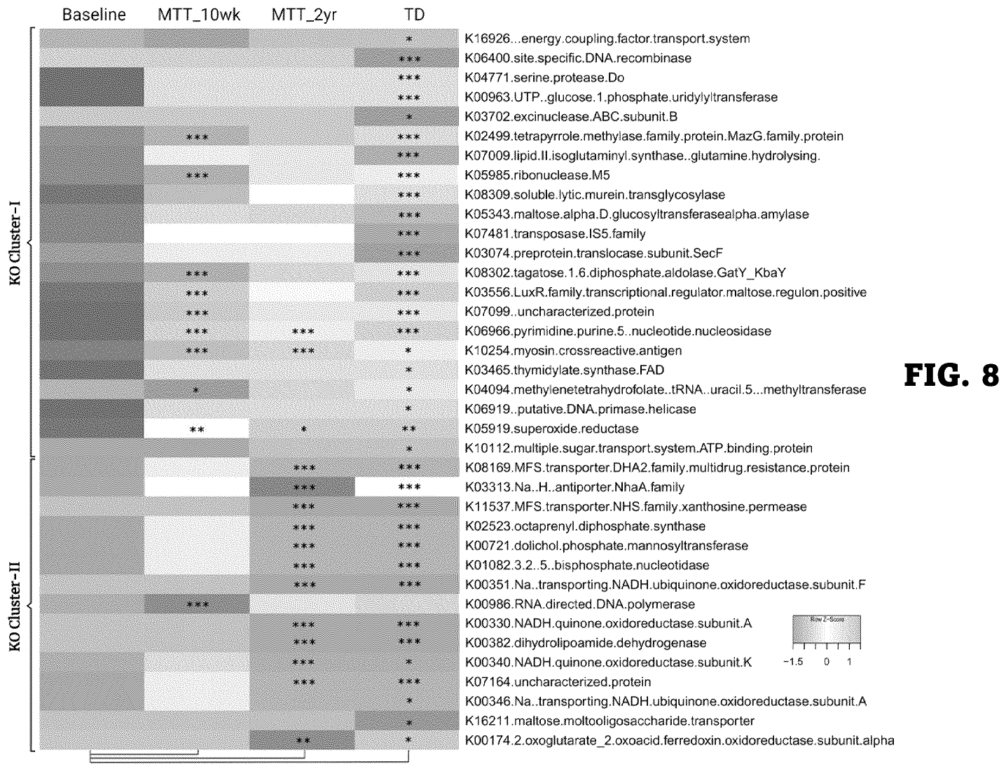

To understand and confirm the KOs shift after MTT, specific differences in KOs between groups were explored. About 5,069 KEGG Orthologs (KOs, functional genes) were identified using HUMAnN2. As shown in FIG. 8, comparing ASD at Baseline vs. TD, 37 KOs that were significantly different (adjusted p<0.05) (Tables 3-5); 22 KOs (out of 37 KOs) were significantly lower (KO Cluster-I), and 15 KOs were higher (KO Cluster-II) (Table 6). KO Cluster-I included KOs that encode genes for oxidative stress response, nucleotide-, carbohydrate-, and protein-degradation, and sulfur metabolism (sulfate reduction). At MTT-10 wk, the relative abundances of 9 of the 37 KOs significantly increased (adjusted p<0.05) compared to ASD baseline and became similar to TD, and none significantly decreased (FIG. 8, Table 6). At MTT-2 yr there was still a general increase compared to ASD Baseline, but only 3 of the 37 KOs increased significantly compared to Baseline, and none decreased significantly.

TABLE 4
Differences in number of taxa between groups
ASD at Baseline vs. MTT-2 yrs (12 significantly increased taxa at MTT-2 yrs

| | |
|---|---|
| Halocynthiibacter_{—}arcticus | Alteromonas_{—}australica |
| Bacteroides intestinalis | Pseudomonas syringae group genomosp.3 |
| Butyricimonas faecalis | Pseudomonas tolaasii |
| Actinomyces pacaensis | Xanthomonas euvesicatoria |
| Dehalobacter restrictus | Streptomyces lividans |
| Candidatus Erwinia haradaeae | Blautia coccoides |

ASD at Baseline vs. MTT-10 wks (16 significantly decreased taxa at MTT-10 wks)

| | |
|---|---|
| Pseudomonas_sp._GR_6-02 | Methylobacterium_{—}oryzae |
| Pseudoalteromonas_{—}issachenkonii | Acetobacter_sp._B6 |
| Mycobacterium_sp._PYR15 | Sphingorhabdus_sp._M41 |
| Bacteroides_{—}xylanisolvens | Pusillimonas_sp._ye3 |
| Alistipes_{—}finegoldii | Enterobacter_{—}cloacae_{—}complex_sp._FDA- |
| | CDC-AR_0132 |
| Synechococcus_sp._PCC_11901 | Leclercia_sp._119287 |
| Serratia ficaria | Xanthomonas perforans |
| Acinetobacter_{—}baylyi | |
| Stenotrophomonas sp. ASS1 | |

TD vs. MTT-2 yrs (6 significantly higher taxa at MTT-2 yrs)

| | |
|---|---|
| Bacteroides intestinalis | Candidatus Erwinia haradaeae |
| Butyricimonas faecalis | Pseudomonas syringae group genomosp.3 |
| Vibrio_sp._EJY3 | Xanthomonas_{—}euvesicatoria |

TABLE 5
Taxonomy and KOs comparison between TD and all ASD groups

| | | | |
|---|---|---|---|
| ASD Baseline | MTT-10 wks | MTT-2 yrs | Differences |

A) Bacterial taxonomy comparisons
vs. TD (comparing all taxa)

| | | | |
|---|---|---|---|
| 0 | 48 | 6 | Higher in ASD vs TD |
| 371 | 387 | 2984 | Lower in ASD vs TD |
| 371 | 435 | 2990 | Total |

B) KOs (KEGG Orthologs) comparisons
vs. TD (comparing all KOs)

| | | | |
|---|---|---|---|
| 15 | 104 | 4 | Higher in ASD vs TD |
| 22 | 40 | 54 | Lower in ASD vs TD |
| 37 | 144 | 58 | Total |

Univariate comparison of the relative abundance (after log₁₀ transformation) of bacterial species (n = 5,272) and KEGG Orthologs (KOs, n = 5,069) between groups. All statistically significant numbers are with adjusted p < 0.05.

TABLE 6
List of significantly different 37 KOs/metabolic pathways in ASD Baseline vs. TD children.

| | | | | | In | P- |
|---|---|---|---|---|---|---|
| No. | Super Pathways | Sub-pathways | KOs | Gene | ASD | values |
| 1 | Carbohydrate | Starch and sucrose | K05343 | treS | Lower | *** |
| | metabolism | metabolism | | | | |
| | | Galactose metabolism | K08302 | gatY-kbaY | | *** |
| 2 | Glycan biosynthesis and | Peptidoglycan | K07009 | gatD | | *** |
| | metabolism | biosynthesis | | | | |
| 3 | Nucleotide/cofactors | Pyrimidine | K03465 | thyX, thy1 | | * |
| | and vitamins | metabolism/One | | | | |
| | Metabolism | carbon pool by | | | | |
| | | folate | | | | |
| | | Pyrimidine metabolism | K06966 | ppnN | | *** |
| 4 | Protein families: | Peptidoglycan | K08309 | slt | | *** |
| | metabolism | biosynthesis and | | | | |
| | | degradation proteins | | | | |
| 5 | Signaling and cellular | Cell growth | K06400 | spoIVCA | | *** |
| | processes | Transporters | K16211 | malY, malT | | * |
| | | | K16926 | htsT | | * |
| 6 | Environmental | Signal transduction | K04771 | degP, htrA | | *** |
| | Information | Membrane transport | K10112 | msmX, | | * |
| | Processing | | | sugC, | | |
| | | | | msiK | | |
| 7 | Genetic information | Chromosome and | K02499 | yabN | | *** |
| | processing | associated proteins | | | | |
| | | Bacterial secretion | K03074 | secF | | *** |
| | | system | | | | |
| | | Transcription factors | K03556 | malT | | *** |
| | | Replication and repair | K03702 | uvrB | | * |
| | | Transfer RNA | K04094 | trmFO, gid | | * |
| | | biogenesis | | | | |
| | | Replication and repair | K06919 | NA | | * |
| | | | K07481 | NA | | *** |
| 8 | Unclassified: | Unclassified | K05919 | dfx | | ** |
| | metabolism | | K05985 | rnmV | | *** |
| | | | K07099 | NA | | *** |
| | | | K10254 | ohyA, sph | | * |

KO Cluster-II significantly higher KOs in ASD Baseline

| | | | | | | |
|---|---|---|---|---|---|---|
| 1 | Carbohydrate | Glycolysis/ | K00174 | korA, | Higher | * |
| | metabolism | Gluconeogenesis/TCA | | oorA, | | |
| | | cycle | | oforA | | |
| | | | K00382 | DLD, lpd, | | *** |
| | | | | pdhD | | |
| | | Pentose and | K00963 | UGP2, | | *** |
| | | glucuronate | | galU, galF | | |
| | | interconversions | | | | |
| 2 | Glycan biosynthesis and | N-Glycan biosynthesis | K00721 | DPM1 | | *** |
| | metabolism | | | | | |
| 3 | Metabolism of | Terpenoid backbone | K02523 | ispB | | *** |
| | terpenoids and | biosynthesis | | | | |
| | polyketides | | | | | |
| 4 | Energy metabolism | Oxidative | K00330 | nuoA | | *** |
| | | phosphorylation | K00340 | nuoK | | * |
| | | Sulfur metabolism | K01082 | cysQ, | | *** |
| | | | | BPNT1 | | |
| 5 | Signaling and cellular | Transporters | K03313 | nhaA | | *** |
| | processes | | K08169 | yebQ | | *** |
| | | | K11537 | xapB | | *** |
| 6 | Unclassified | Unclassified | K00346 | nqrA | | * |
| | | | K00351 | nqrF | | *** |
| | | | K00986 | ltrA | | *** |
| | | | K07164 | NA | | *** |

* Single asterisk indicates p < 0.05, triple ''' asterisks indicate p < 0.001, NA-gene name not available. All p-values are FDR corrected.

KO Cluster-II (FIG. 8) consist of 15 KOs including KOs that encode for ion and sugar transporters, carbohydrate degradation and energy production, and terpenoids and polyketides biosynthesis (Table 4). These KOs were higher at Baseline compared to TD. At MTT-10 wk, one KO significantly decreased, 8 decreased but were not statistically significant, and none significantly increased. At MTT-2 yr 12 of the 15 KOs that were higher at Baseline significantly decreased (adjusted p<0.05) and became more similar to TD, and none had significantly increased (FIG. 8, Table 4). Overall, after MTT (10 wk, 2 yr), the relative abundance of KOs from Cluster-I and -II became more similar to TD. These findings suggest that MTT had a positive functional impact on children with ASD and it shifted the functional-gene profile of those genes towards the profile of TD children.

For all KOs, longitudinal pairwise comparison between Baseline and MTT-10 wks showed 183 KOs were significantly different (adjusted p<0.05); 149 KOs were significantly increased and 34 were decreased after MTT. At MTT-2 yrs, 209 KOs were significantly different; 13 KOs were increased but 196 were decreased (Table 1). Comparison of TD vs. MTT (10 wks, 2 yr) for KOs showed similar trend like ASD Baseline vs. MTT (Table 1). So, major changes in the metabolic pathways occurred at MTT-10 wks, but additional major and distinct changes occurred at 2 years post-treatment.

Again, considering all KOs, comparing TD vs. MTT-10 wks, 144 KOs were significantly different (104 KOs were higher and 40 were lower, adjusted p<0.05). For TD vs. MTT-2 yrs, 58 KOs were significantly different (4 KOs were higher and 54 were lower at MTT-2 yrs, adjusted p<0.05) (Table 2-3). Overall, the KOs became more dissimilar between TD and MTT-10 wks (144 different KOs) compared to ASD Baseline (37 different KOs) and remained somewhat more dissimilar at MTT-2 yrs (58 different KOs) (Tables 2-3).

### Example 3: Microbial Functional Genes Shifted with MTT

To address the global KEGG Orthologs (KOs) changes before and after MTT (10 wk, 2 yr), alpha and beta diversity indices were used. Shannon index significantly increased after MTT-10 wk compared to ASD Baseline (FIG. 2B), but after MTT-2 yr, alpha diversity did not change significantly. There was no significant difference in diversity between TD and Baseline. Similar to the taxa analysis, KOs be-ta-diversity was also significantly distinct for the Jaccard dissimilarity index between MTT-2 yr (ANOSIM R=0.32, p=0.001) and other groups (FIG. 2B, Table 1), suggesting that after 2 years of MTT, rare/less abundant KOs significantly changed in children with ASD compared to Baseline.

To understand and confirm the KOs shift after MTT, we explored specific differences in KOs between sample groups. We identified 5069 KEGG Orthologs (KOs, functional genes) using HUMAnN2. As shown in FIG. 8, comparing ASD at Baseline vs. TD, 37 KOs were significantly different (adjusted p<0.05) (Table 3, 5); 22 KOs were significantly lower (KO Cluster-I), and 15 KOs were significantly higher (KO Cluster-II) (Table 6). For KO Cluster-I, nine of the KOs increased at MTT-10 wk and three remained increased at MTT-2 yr. For KO Cluster-II, 1 KO decreased at MTT-10 wk and 12 decreased at MTT-2 yr. Thus, MTT resulted in normalizing many KOs that were initially lower or higher in ASD and became closely similar to TD.

### Example 4: Relative Abundance of Important Metabolic Genes Changes after MTT

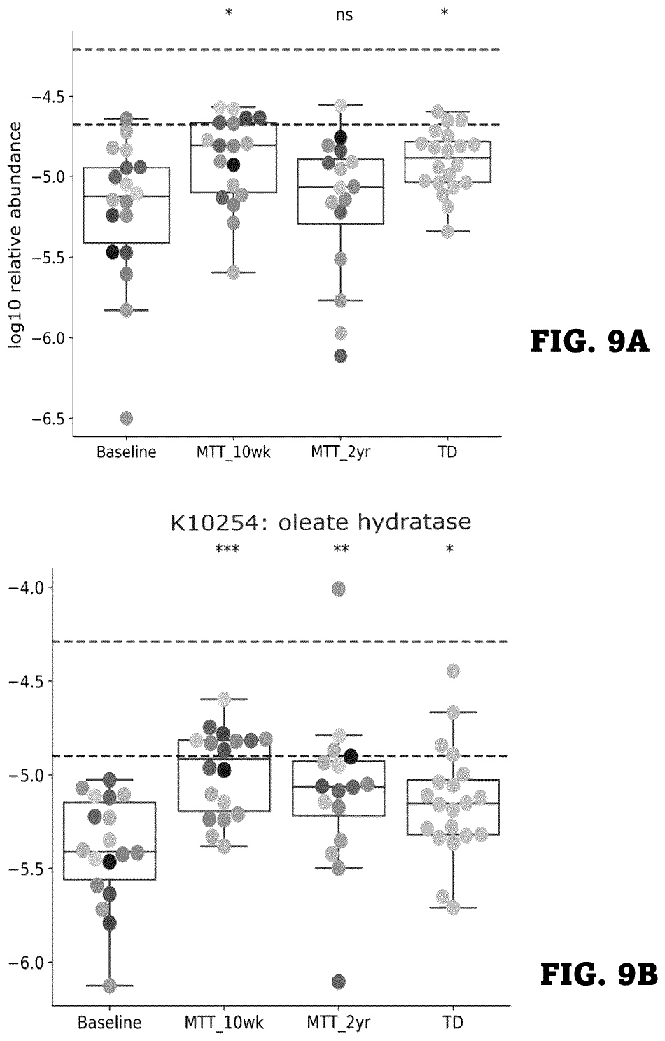

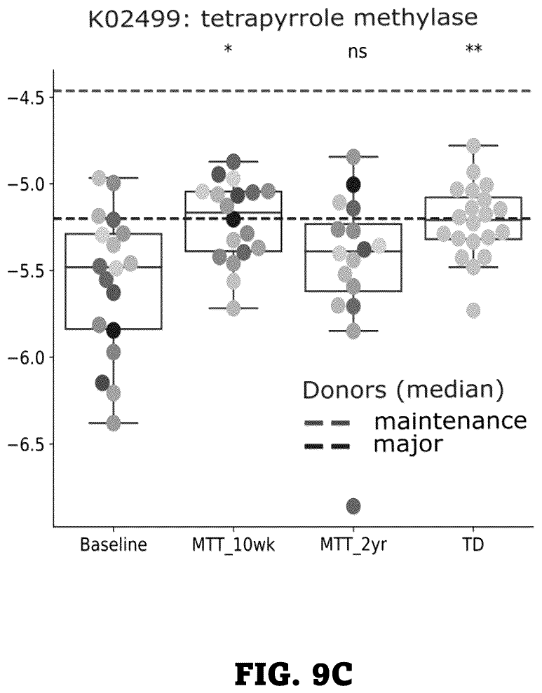

Details on KOs that were significantly different between ASD and TD (adjusted p<0.05), or significantly changed after MTT (adjusted p<0.05), and have been previously potentially linked to ASD are provided here. Table 6 lists the metabolic pathways associated with each gene. A closer look at KO Cluster-I (FIG. 9A-C) shows changes in relative abundance for genes encoding for: folate biosynthesis (K04094), vitamin B12 synthesis (K02499), and oleic acid synthesis (K10254: omega-9-fatty acids). As seen in FIG. 9, the relative abundance of these genes was significantly lower in ASD at Baseline vs. TD and significantly increased (adjusted p<0.05) at MTT-10 wk and became similar to TD and closer to donor levels. At MTT-2 yr, K04094 and K02499 non-significantly decreased (median, p>0.05), but K10254 remained significantly higher than baseline.

### Example 5: Abundance of Genes Encoding for Oxidative Stress Protection and Sulfur Metabolism Changed after MTT

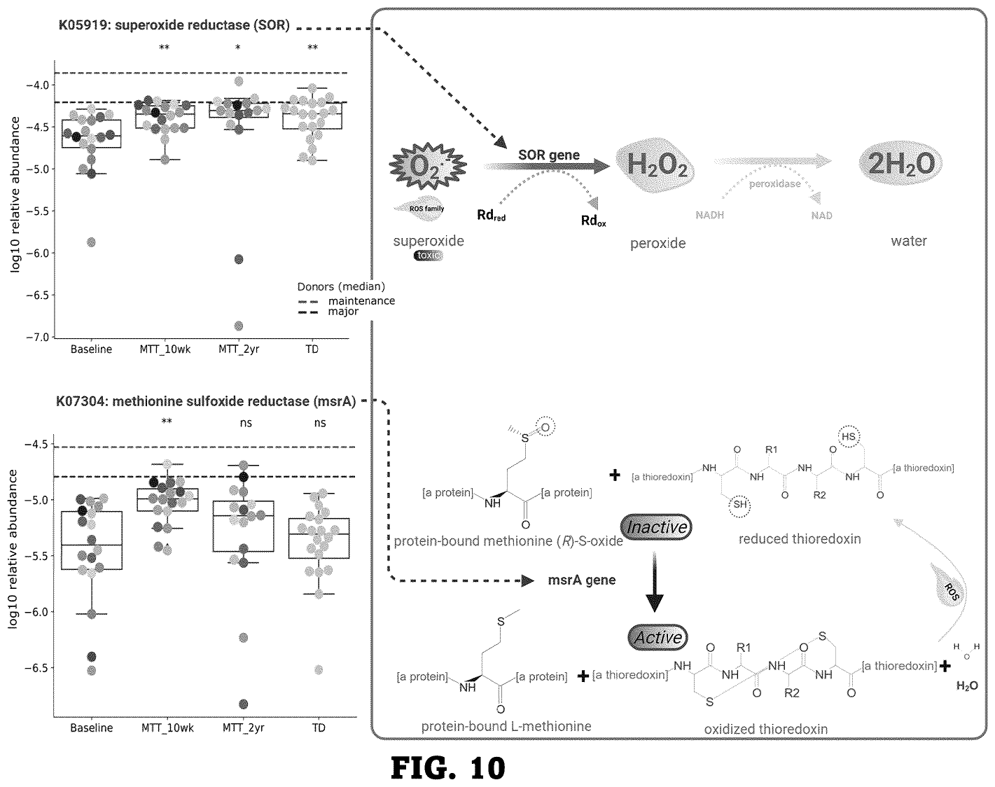

Functional gene analyses of the gut microbiome also revealed that MTT might support the microbial ecosystem by increasing the abundance of microbes that can detoxify oxygen, and microbes with enzymes that protect against oxidative stress. As seen in FIGS. 8 and 10, the relative abundance of the gene that encodes for K05919 (dfx gene, SOR): superoxide reductase was significantly lower in ASD at Baseline, and significantly increased (adjusted p<0.05) at MTT-10 wk and -2 yr vs. Baseline, and became more similar to the TD group.

This SOR microbial gene encodes for an enzyme that converts toxic superoxide to peroxide, which is subsequently reduced to water, and can help to neutralize ROS similarly to superoxide (FIG. 10, top). Another KO responsible for oxidative stress protection is K07304 (msrA): peptide-methionine (S)-S-oxide reductase (FIG. 10). ROS inactivates the sulfur amino acid “methionine-sulfur-oxide”, but KO K07304 (msrA) activates methionine-sulfur-oxide by reducing it and oxidizing thioredoxin (FIG. 10, bottom). The relative abundance of KO K07304 (msrA) was non-significantly lower at Baseline vs. TD (median, p>0.05), and significantly increased at MTT-10 wk (adjusted p<0.05) (FIG. 10, bottom), but there was no significant difference (median remained higher) for MTT-2 yr against Baseline. FIG. 10 also shows the enzymatic reaction of K05919 (SOR) and K07304 (msrA gene) enzymes for oxygen detoxification and oxidative stress protection after MTT, respectively.

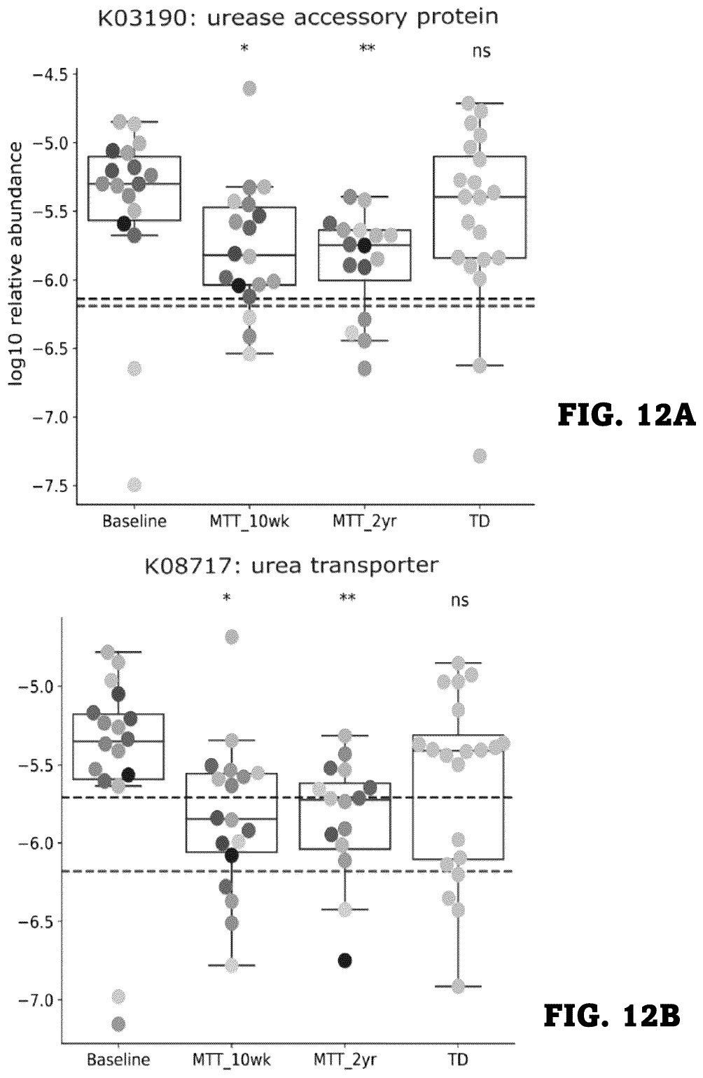

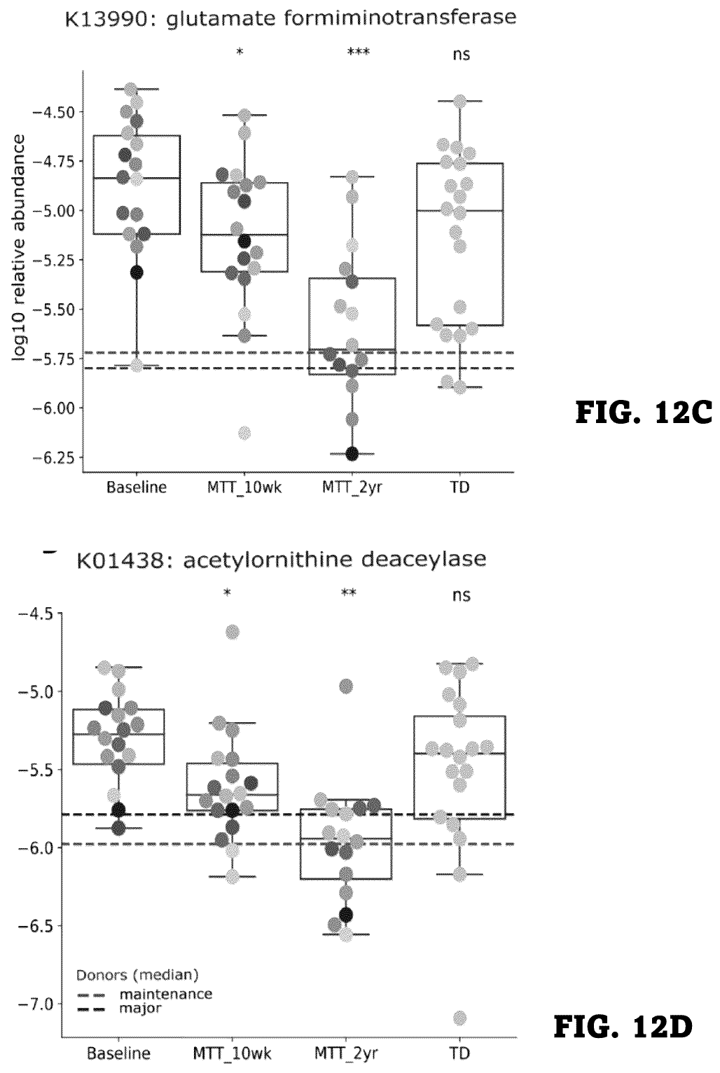

Differences in the relative abundances of important functional genes that were not significantly different between Baseline and TD but changed significantly (adjusted p<0.05) after MTT (10 wk/2 yr) were also explored. For example, K03190 (ureD, ureH): urease accessory protein (FIG. 12A), K08717 (utp): Urea transporter (FIG. 12B), K13990 (FTCD): glutamate formiminotransferase (produce glutamate from histidine) (FIG. 12C), and K01438 (argE): acetylornithine deacetylase (ornithine and arginine biosynthesis) (FIG. 12D) were non-significantly higher at Baseline compared to TD but significantly decreased after MTT (10 wk, 2 yr) (FIG. 12). One possible reason for a decrease in abundance of these genes and closer to donors after MTT is that major and maintenance donors (median) abundance were also lower than Baseline and TD.

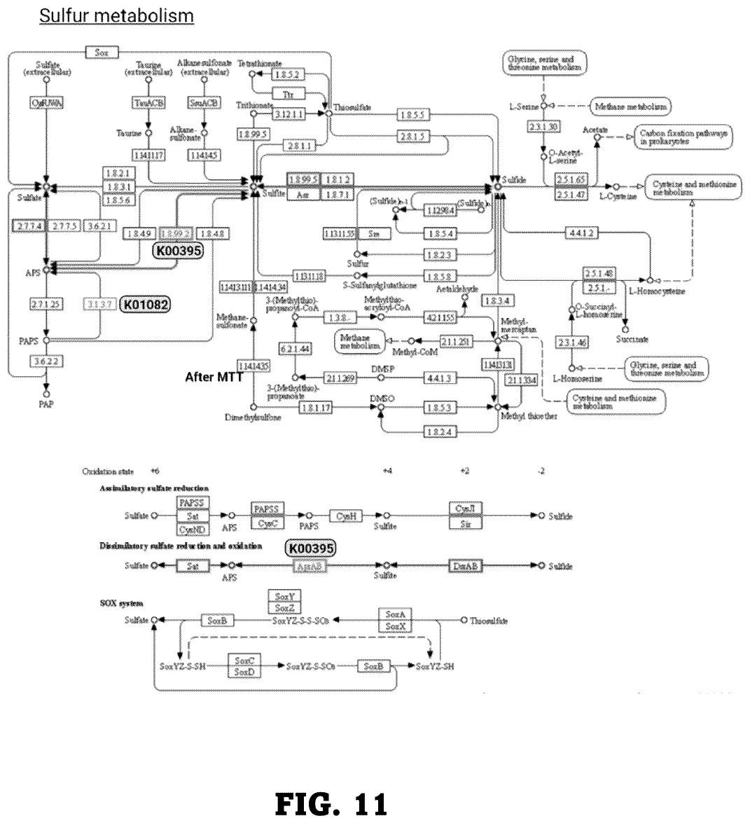

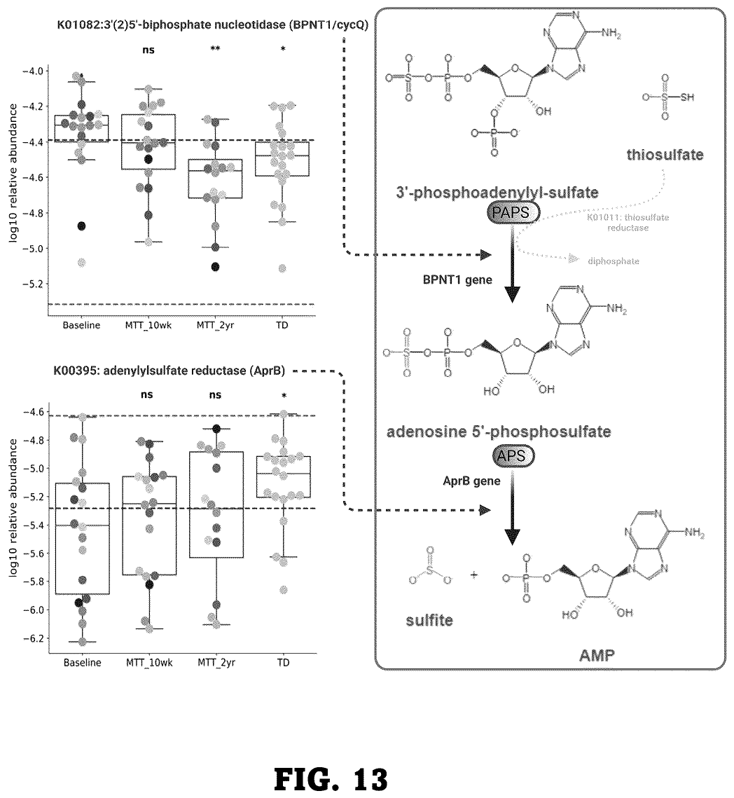

3′-Phosphoadenosine-5′-phosphosulfate (PAPS) and adenosine-5′-phosphosulfate (APS) are important phosphosulfate compounds for sulfur metabolism that participate in assimilatory and dissimilatory sulfate reduction via sulfate-reducing bacteria (FIG. 11). As shown in FIG. 13, the relative abundance of the gene that encodes for KO K01082 (BPNT1/cycQ) 3′(2′), 5′-bisphosphate nucleotidase, the enzyme that converts PAPS to APS, was significantly higher at Baseline and non-significantly decreased (median, p>0.05) at MTT-10 wk, and decreased significantly at MTT-2 yr vs. Baseline (adjusted p<0.05) and its relative abundance became more similar to TD (FIG. 13, top).

The relative abundance of the gene that encodes for KO K00395 (aprB) adenylylsulfate/APS reductase, subunit B (the enzyme that converts APS to sulfite), was significantly lower in ASD Baseline vs. TD (adjusted p<0.05), and non-significantly increased after MTT-10 wk and -2 yr (median, p>0.05), and became more similar to the TD group (FIG. 8B).

FIG. 13 illustrates dissimilatory sulfur reduction and the contribution of BPNT1 and aprB to the process.

We also explored differences in the relative abundances of important functional genes that were not significantly different between Baseline and TD but changed significantly after MTT-10 wk and -2 yr (adjusted p<0.05) (FIG. 12A-D).

### Example 6: Correlation Analysis Shows Links Between Omics and GSRS

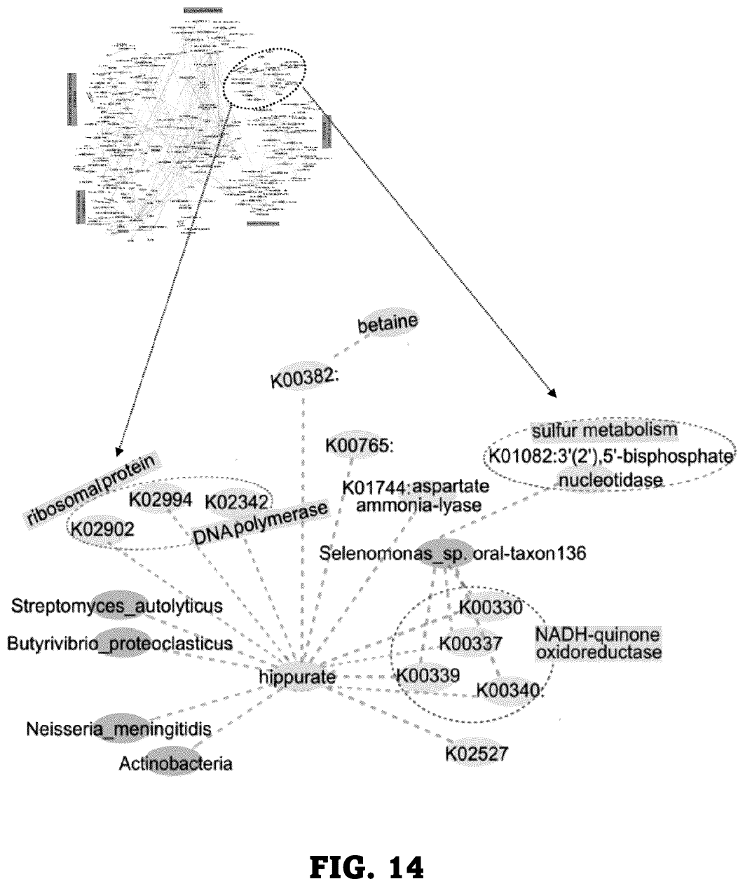

Correlation analysis was used to uncover the potential relationship of bacterial taxonomy, bacterial genes, plasma metabolites, and ASD and GI symptom data (FIG. 14). Within the correlation network, 280 nodes were identified with 420 edges with correlations of R>0.6 or R<−0.6, and adjusted p<0.05. Network analysis was performed only for the ASD Baseline group and only included blood metabolites, not fecal metabolites, as we did not observe significant changes in fecal metabolites after treatment.

The correlation network showed 241 positive (green color) and 179 negative (red) interactions/correlations for genes (KOs) related to neurotransmitters/neuroactive molecules, amino acids, indole, taurine, tyramine derivatives, and for sulfur metabolism (FIG. 14). Significant correlation networks for Prevotella, Bifiobacterium, or Desulfovibrio species were not observed, or important KOs for oxidative stress, dissimilatory sulfate reduction or other taxa/KOs mentioned in this section.

### Example 7: Discussion and Conclusion

The current analysis shows improvement in symptoms for ASD (GI) with MTT as a result of remodeling of the gut microbiome to resemble more gut microbiomes of healthy donors or TD/controls. Taxonomic analysis showed many lower abundances of taxa at Baseline that increased after MTT-10 wk (FIG. 4). Interestingly, after 2 years, the taxonomic composition became distinct to the other groups analyzed (FIGS. 2B and 4), suggesting MTT changed the microbial composition, but over time these children developed different/own microbial composition.

Previous 16S rRNA gene amplicon measurements of the same samples used in this study showed a lower abundance of the genera Prevotella, Bifidobacterium and Desulfovibrio before treatment, and major improvements after MTT. At Baseline, Prevotella sp. abundance compared to TD (FIG. 5) was lower, although not statistically significantly (p>0.05), but the relative abundance increased significantly at MTT-10 wk vs. Many Prevotella species were identified in fecal samples, but some of them were similar to oral Prevotella species, such as P. dentalis (FIG. 5) and P. denticola (FIG. 7A). It was hypothesized that these oral Prevotella may have transferred from mouth to gut more than normal because of the use of a proton pump inhibitor in our MTT study. Many gut Prevotella species are fiber-consuming bacteria. One possible reason for Prevotella's depletion in children with ASD in the USA could be lower fiber content in the westernized diet leading to the depletion of bacteria with fiber-degrading enzymes, and this could contribute to poorer GI health and behaviors in children with ASD.

Similarly, two probiotic examples of Bifidobacterium (B. bifidum FIG. 6A; B. angulatum, FIG. 7E) and a sulfur-reducer (Desulfovibrio piger, FIG. 7B) significantly increased at MTT-10 wk vs. Baseline and their relative abundance became comparable to TD (FIGS. 6A and 6B). The abundance of these microbes increased non-significantly (median) at MTT-2 yr compared to Baseline (p>0.05) (FIGS. 6A and 6B). Bifidobacteria are SCFA producers, and “psychobiotics”, and they modulate the gut-brain signals via y-aminobutyric acid (GABA) and glutamate metabolism. Bifidobacterium improve behavior and prevent depression-like behaviors in mice. Desulfovibrio is a sulfur-reducing bacteria (SRB) that can degrade mucins, SCFAs, and amino acids (glutamate, alanine) in the human colon, and may help to maintain the integrity of the gut epithelium. However, some reports have shown a seemingly different result: higher Desulfovibrio in ASD Baseline compared to TD fecal samples. It is important to note that Prevotella, Bifidobacterium and Desulfovibrio species were not significantly different between ASD Baseline and TD, although the median was lower for Baseline than TD. After MTT (10 wk), the abundance of these bacteria significantly increased and moved closer to the maintenance donor (dashed-red horizontal line) (FIGS. 5 and 6).

The relative abundance of Lactobacillus vaginalis increased significantly at MTT-10 wk vs. Baseline and became more similar to TD (FIG. 6C). L. vaginalis is a common vaginal commensal, and has also been reported in human feces and oral cavities. Similar to Bifidobacterium, the genus Lactobacillus are also “psychobiotics” and can modulate the gut-brain connection via neuroactive molecules (GABA, glutamate).

A higher relative abundance of Alistipes species has been reported in children with autism, pervasive developmental disorder not otherwise specified (PDD-NOS), and depression. Interestingly, the relative abundance of Alistipes finegoldii was significantly lower after MTT (10 wk and 2 yr) compared to Baseline (FIG. 6D) and became comparable to TD. It is important to note that, after MTT (10 wks), bacterial abundance changed, but over time (2 yrs) returned closer to Baseline levels for all presented taxa except Alistipes and Desulfovibrio (FIGS. 5 and 6). This finding suggests that the impact of MTT treatment in these bacteria were temporary and longer treatment may have been needed. Alistipes and Desulfovibrio remained significantly changed after 2 years of MTT (FIG. 6).

At the global level of KOs, it was observed that many microbial genes had altered levels at Baseline compared to TD, but after MTT, microbial gene abundance became similar to TD or closer to healthy donors (FIG. 8). This finding suggests that MTT had a positive impact on ASD children and helped to restore the metabolic pathways. To understand this more closely, KOs were examined. Interestingly, trmFO (K04094), the gene that encodes for the tetrahydrofolate-synthesizing enzyme, was at lower abundance in Baseline vs. TD, but after MTT, its relative abundance significantly increased and became more similar to the TD group (FIG. 9A). This suggests that microbes enhanced by MTT have the potential to synthesize folate for the host, and folate supplementation has been demonstrated to improve ASD symptoms.

Oxidative stress is a major concern in ASD etiology and it can disrupt neuron connections in the brains of individuals with ASD by causing neuroinflammation and cognitive impairment. Imbalance in redox reactions and a lack of antioxidants such as folinic acid, glutathione, vitamins (C and E) or coenzymes NAD⁺/NADH are possible contributing factors for ROS in ASD. Interestingly, the relative abundance of genes that encode for K05919: superoxide reductase (dfx gene, SOR) was low at Baseline vs. TD, but significantly increased after MTT (10 wk, 2 yr), and its abundance became similar to TD (FIG. 10). This microbial SOR converts highly reactive and toxic superoxide (O₂-) to less toxic hydrogen peroxide (H₂O₂), which is subsequently converted to H₂O. SOR is present in anaerobic microbes such as SRB, e.g., Desulfovibrio. Interestingly, it was found that the relative abundance of Desulfovibrio piger was low at Baseline but significantly increased after MTT (FIG. 6B).

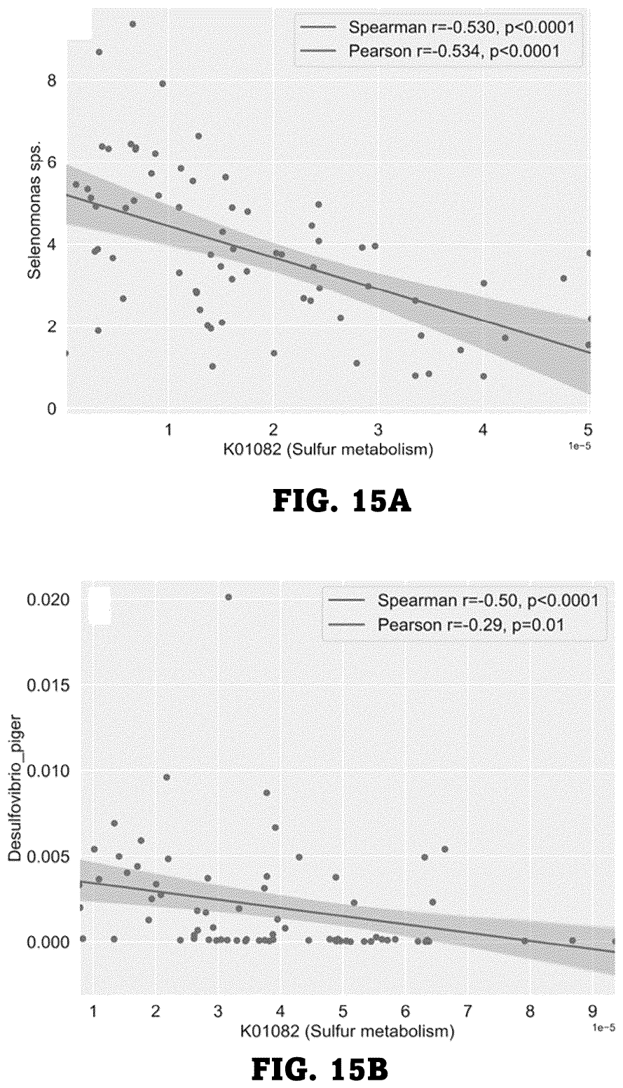

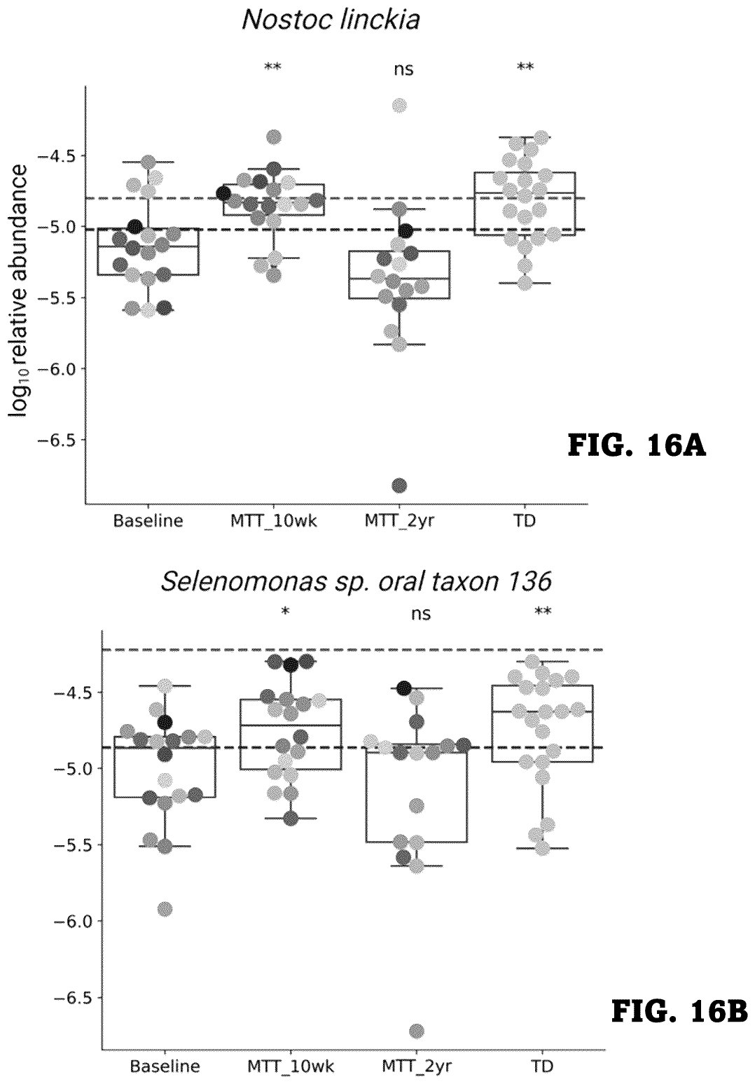

Differences in relative abundance in genes that encode for two KOs that participate in microbial dissimilatory sulfate reduction: K01082 (BPNT1/cycQ) 3′(2′), 5′-bisphosphate nucleotidase and K00395 adenylylsulfate reductase, subunit B (aprB gene) were observed (FIG. 11, FIG. 13). A significant negative correlation of K01082 (BPNT1/cycQ) with sulfur-reducing bacteria Selenomonas species was observed (FIGS. 15A and 16B (relative abundance) and with Desulfovibrio piger (FIG. 15B), although the effect size was small. These findings suggest an imbalance in microbial sulfate reduction in children with ASD, that is normalized by MTT, bringing these KOs levels close to the levels observed in TD. The findings further suggest that ASD at Baseline has an altered abundance of microbial genes for dissimilatory sulfate reduction (sulfur metabolism, FIG. 11). Another interesting finding was that after MTT, the relative abundance of the functional genes in ASD children became more similar to the relative abundance of the donors.

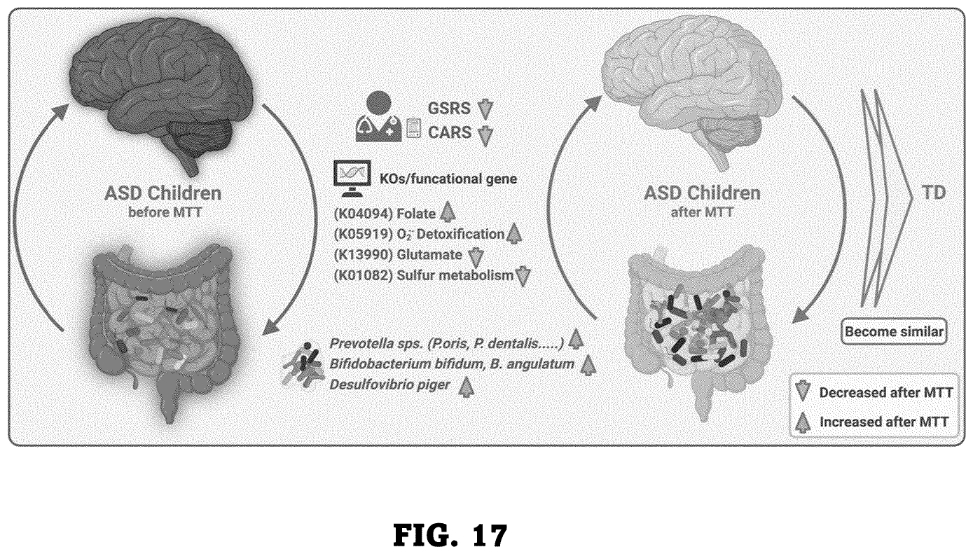

Correlations of bacteria with GI and ASD symptoms were investigated. Nostoc linckia, a cyanobacteria, was positively correlated with GSRS at Baseline (FIG. 17A). It has been suggested that some Nostoc species can produce P-N-methylamino-L-alanine (BMAA) and target the gut immune system and cause chronic low-grade inflammation. However, no significant correlations between CARS and microbiota were observed in this study.

A strength of this study is that it is the first study exploring the gut microbiome and metabolic pathways in children with ASD before and after MTT using shotgun metagenomic data analyses. This study provides higher taxonomic classification and an indication of microbial pathways that might be important to achieve improvements through microbial interventions such as MTT. k-mer (short sequencing fragments) alignment was used to process sequences and the marker gene database was used for taxonomic and functional analysis. Three of the eighteen children did make changes in their diet in the two years after MTT treatment ended. Since ASD and GI symptoms improved after MTT, it shows that MTT led the primary improvement in symptoms in children with ASD, and diet might have a complementary impact after children are more comfortable.

The current findings show that MTT in ASD children changed the microbial composition by normalizing levels of many bacteria that were initially low. MTT also increased the abundance of previously detected beneficial bacterial such as Prevotella, Bifidobacterium, and sulfur-reducer Desulfovibrio at the species level, but over the time (2 yrs) the abundance of Prevotella and Bifidobacterium decreased, which suggests a longer MTT treatment time or a booster after a certain time might be needed for the retention of these bacteria. Similarly, MTT also resulted in normalizing the levels of many bacterial genes (KOs). Interestingly, microbial metabolic genes (KOs) for folate biosynthesis, oxidative stress protection and sulfur metabolism were different at ASD Baseline than TD, but after MTT (10 wk, 2 yr), they became more similar to the TD and/or donor levels (FIG. 17).

1. A method for monitoring effectiveness of a treatment of an autism spectrum disorder (ASD) in a subject in need thereof, comprising:
a) obtaining or having obtained a stool sample from the subject during or after a treatment period with a fecal microbiota preparation;
sequencing the stool sample or portion thereof, wherein the sequencing comprises a shotgun metagenomic sequencing;
b) determining a relative abundance level of at least one microorganism in the subject's gut microbiome using data from the shotgun metagenomic sequencing;
c) comparing the relative abundance level of the at least one microorganism in the subject's gut microbiome with an abundance baseline; wherein the abundance baseline is the abundance of the at least one microorganism in a baseline stool sample obtained from the subject prior to the treatment period or obtained from a subject not diagnosed with ASD;
and
d) confirming the effectiveness of the treatment when the relative abundance level of the at least one microorganism is increased as compared to that of the abundance baseline.

2. The method of claim 1, wherein the baseline stool sample is obtained from or having been obtained from the subject prior to the treatment period.

3. The method of claim 1, wherein the baseline stool sample is obtained from or having been obtained from a subject not diagnosed with ASD.

4. (canceled)

5. The method of claim 1, wherein the at least one microorganism is selected from a group consisting of Prevotella dentalis, Prevotella enoeca, Prevotella oris, Prevotella meloninogenica, Prevotella denticola, Prevotella fusca, Prevotella intermedia, Prevotella ruminicola, Bifidobacterium bifidum, Bifidobacterium angulatum, Selenomonas sp. Selenomonas sp. oral taxon 136, Lactobacillus vaginalis, Alistipes finegoldii, Desulfovibrio sp., Desulfovibrio piger, and any combination thereof.

6. The method of claim 1, wherein the treatment period is from 10 weeks to about 2 years.

7. (canceled)

8. The method of claim 1, wherein the steps a)-c) are implemented using computer software.

9. The method of claim 1, wherein the treatment comprises Microbiota Transfer Therapy (MTT).

10. A method for monitoring effectiveness of a treatment of an autism spectrum disorder (ASD) in a subject in need thereof, comprising:
a) obtaining or having obtained a stool sample from the subject during or after a treatment period with a fecal microbiota preparation, and
sequencing the stool sample or portion thereof, wherein the sequencing comprises a shotgun metagenomic sequencing;
b) determining a relative gene abundance level of at least one metabolic gene in the stool sample using the shotgun metagenomic sequencing data;
c) comparing the relative gene abundance level of the at least one gene with a gene abundance baseline of the at least one gene wherein the gene abundance baseline of the at least one gene is the gene abundance of the at least one gene in a baseline stool sample from the subject prior to the treatment period or from a subject not diagnosed with ASD
and
d) confirming the effectiveness of the treatment when
(i) the relative gene abundance level of the at least one metabolic gene involving in folate biosynthesis and/or oxidative stress protection is increased as compared to that of the gene abundance baseline; or
(ii) the relative gene abundance level of the at least one metabolic gene involving in glutamate metabolism and/or sulfur metabolism is decreased as compared to that of the gene abundance baseline.

11. The method of claim 10, wherein the baseline stool sample is obtained or having been obtained from the subject prior to the treatment.

12. The method of claim 10, wherein the baseline stool sample is obtained or having been obtained from a subject not diagnosed with ASD.

13. (canceled)

14. The method of claim 10, wherein the at least one metabolic gene is selected from a group consisting of a folate biosynthesis (K04094), vitamin B-12 synthesis (K02499), oleic acid synthesis (K10254), sulfur metabolism (dissimilatory sulfate reduction) aprB (K00395), an oxidative stress protection gene (K05919, K07304), ureH, ureD (K03190), urea transporter utp (K08717), glutamate metabolism FTCD (K13990), ornithine and arginine biosynthesis argE (K01438)_, and any combination thereof.

15. (canceled)

16. The method of claim 10, wherein the at least one metabolic gene is selected from the group consisting of ureH, ureD (K03190), utp(K08717), FTCD(K13990), or argE(K01438), and any combination thereof.

17. The method of claim 10, wherein the treatment period is from about 5 weeks to about 5 years.

18. (canceled)

19. The method of claim 10, wherein the steps a)-c) are implemented using computer software.

20. A method for treating an autism spectrum disorder (ASD) in a subject in need thereof, comprising:
a) obtaining or having obtained a stool sample from the subject during or after a treatment period with a fecal microbiota preparation; and
sequencing the stool sample or portion thereof, wherein the sequencing comprises a shotgun metagenomic sequencing;
b) determining a relative abundance level of at least one microorganism in the subject's gut microbiome using data from the shotgun metagenomic sequencing;
c) comparing the relative abundance level of the at least one microorganism in the subject's gut microbiome with an abundance baseline, wherein the abundance baseline is the abundance of the at least one microorganism in a baseline stool sample obtained from the subject prior to the treatment period or obtained from a subject not diagnosed with ASD;
and
d) confirming the effectiveness of the treatment when the relative abundance level of the at least one microorganism is increased as compared to that of the abundance baseline.

21. (canceled)

22. The method of claim 20, wherein the at least one microorganism is selected from the group consisting of Prevotella dentalis, Prevotella enoeca, Prevotella oris, Prevotella meloninogenica, Prevotella denticola, Prevotella fusca, Prevotella intermedia, Prevotella ruminicola, Bifidobacterium bifidum, Bifidobacterium angulatum, Selenomonas sp. Selenomonas sp. oral taxon 136, Lactobacillus vaginalis, Alistipes finegoldii, Desulfovibrio sp., Desulfovibrio piger, and any combination thereof.

23. The method of claim 20, further comprising:
e) determining a relative gene abundance level of at least one metabolic gene in the stool sample using the shotgun metagenomic sequencing data;
f) comparing the relative gene abundance level of the at least one metabolic gene with an abundance baseline of the at least one metabolic gene, wherein the abundance baseline of the at least one metabolic gene is the abundance of the at least one metabolic gene in a baseline stool sample from the subject prior to the treatment period or from a subject not diagnosed with ASD;
and,
g) confirming the effectiveness of the treatment when
(i) the relative gene abundance level of the at least one metabolic gene involving in folate biosynthesis and/or oxidative stress protection is increased as compared to that of the abundance baseline; or
(ii) the relative gene abundance level of the at least one metabolic gene involving in glutamate metabolism and/or sulfur metabolism is decreased as compared to that of the abundance baseline.

24. The method of claim 23, wherein the at least one metabolic gene is selected from the group consisting of folate biosynthesis (K04094), vitamin B-12 synthesis (K02499), oleic acid synthesis (K10254), sulfur metabolism (dissimilatory sulfate reduction) aprB (K00395), an oxidative stress protection gene (K05919, K07304), ureH, ureD (K03190), urea transporter utp (K08717), glutamate metabolism FTCD (K13990), ornithine and arginine biosynthesis argE (K01438), and any combination thereof.
